(19) United States
(12) Patent Application Publication
(10) Pub. No.: US 2014/0193408 A1
(43) Pub. Date: Jul. 10, 2014

(54) SOLUBLE PROTEINS FOR USE AS THERAPEUTICS

(71) Applicants: Thomas Huber, Allschwil (CH); Frank Kolbinger, Neuenburg (DE); Karl Welzenbach, Huningue (FR)

(72) Inventors: Thomas Huber, Allschwil (CH); Frank Kolbinger, Neuenburg (DE); Karl Welzenbach, Huningue (FR)

(73) Assignee: NOVARTIS AG, Basel (CH)

(21) Appl. No.: 14/126,223

(22) PCT Filed: Jun. 15, 2012
(86) PCT No.: PCT/IB2012/053040

§ 371 Date: Mar. 6, 2014

Related U.S. Application Data
(60) Provisional application No. 61/625,713, filed on Apr. 18, 2012.
(60) Provisional application No. 61/497,668, filed on Jun. 16, 2011.

(51) Int. Cl. C07K 16/28; C07K 14/705

(52) U.S. Cl. CPC C07K 16/2896; C07K 14/70596
USPC 424/134.1; 530/387.3; 536/23.4; 435/328; 435/69.6

(57) ABSTRACT

The present invention relates to improved binding proteins, for use as a medicament, in particular for the prevention or treatment of autoimmune and inflammatory disorders, for example allergic asthma and inflammatory bowel diseases. The invention more specifically relates to a soluble protein, comprising a complex of two heterodimers, wherein each heterodimer essentially consists of: (i) a first single chain polypeptide comprising: (a) an antibody heavy chain sequence having VH, CH1, CH2, and CH3 regions; and (b) a monovalent region of a mammalian binding molecule fused to the VH region; and (ii) a second single chain polypeptide comprising: (c) an antibody light chain sequence having a VL and CL region; and (d) a monovalent region of a mammalian binding molecule fused to the VL region; characterised in that each pair of VH and VL CDR sequences has specificity for an antigen, such that the total valency of said soluble protein is six. The invention further relates to soluble SIRPa-binding antibody-like proteins as shown in FIG. 1.

The present invention relates to soluble, multispecific, multivalent binding proteins, for use as a medicament, in particular for the prevention or treatment of autoimmune and inflammatory disorders, for example allergic asthma and inflammatory bowel diseases. The soluble proteins of the invention comprise a complex of two heterodimers, wherein each heterodimer essentially consists of:

(i) a first single chain polypeptide comprising:
(a) an antibody heavy chain sequence having VH, CH1, CH2, and CH3 regions; and
(b) a monovalent region of a mammalian binding molecule fused to the VH region; and
   (ii) a second single chain polypeptide comprising:
(c) an antibody light chain sequence having a VL and CL region; and
(d) a monovalent region of a mammalian binding molecule fused to the VL region;
   characterised in that each pair of VH and VL CDR sequences has specificity for an antigen, such that the total valency of said soluble protein is six.

The invention more specifically relates to soluble binding proteins having specificity for SIRPα. One specific embodiment of the invention is further illustrated by FIG. 1.

SIRPα (CD172a) is an immunoreceptor expressed by myeloid lineage cells including macrophages, granulocytes and conventional dendritic cells (DCs), as well as on neuronal cells (van den Berg, et al. 2008, Trends in Immunol., 29(5):203-6). SIRPα is a low affinity ligand for CD47 (Rebres, et al. 2001, J. Biol. Chem.; 276(37):34607-16; Hatherley, et al. 2007; J. Biol. Chem.; 282(19):14567-75; Hatherley, et al. 2008; Mol. Cell; 31(2) 266-77) and the interaction of SIRPα with CD47 composes a cellular communication system based on adhesion and bidirectional signaling controlling, which regulates multiple cellular functions in the immune- and neuronal system. These functions include migration, cellular maturation, macrophage phagocytosis and cytokine production of myeloid dendritic cells (van den Berg, et al. 2008 Trends in Immunol. 29(5):203-6; Sarfati 2009, Current Drug Targets, 9(10):852-50).

Data from animal models suggest that the SIRPα/CD47 interaction may contribute to or even control the pathogenesis of several disorders including autoimmune, inflammatory (Okuzawa, et al. 2008, BBRC; 371(3):561-6; Tomizawa, et al. 2007, J Immunol; 179(2):869-877); ischemic (Isenberg, et al. 2008, Arter. Thromb Vasc. Biol., 28(4):615-21; Isenberg 2008, Am. J. Pathol., 173(4):1100-12) or oncology-related (Chan, et al. 2009, PNAS, 106(33): 14016-14021; Majeti, et al. 2009, Cell, 138(2):286-99) diseases. Modulating the SIRPα/CD47 pathway may therefore be a promising therapeutic option for multiple diseases.

The use of antibodies against CD47, SIRPα or CD47-derived SIRPα-binding polypeptides has been suggested as therapeutic approaches (see for example WO 1998/40940, WO 2004/108923, WO 2007/133811, and WO 2009/046541). Besides, SIRPα binding CD47-derived fusion proteins were efficacious in animal models of disease such as TNBS-colitis (Fortin, et al. 2009, J Exp Med., 206(9):1995-2011), Langerhans cell migration (J. Immunol. 2004, 172: 4091-4099), and arthritis (VLST Inc, 2008, Exp. Opin. Therap. Pat., 18(5): 555-561).

In addition, SIRPα/CD47 is suggested to be involved in controlling phagocytosis (van den Berg, et al. 2008, Trends in Immunol., 29(5):203-6) and intervention by SIRPα binding polypeptides was claimed to augment human stem cell engraftment in a NOD mouse strain (WO 2009/046541) suggesting the potential benefits of CD47 extracellular domain (ECD) containing therapeutics for use in human stem cell transplantation.

The present invention provides soluble binding proteins comprising heterodimers of first and second polypeptide chains, each chain comprising a binding moiety fused to an antibody heavy or light chain sequence. The soluble proteins can have mono-, bi- tri- or quad-specificity for an antigen, target, or binding partner, and an increased valency compared to prior art molecules. Compared to prior art molecules the soluble proteins of the invention provide an increased number of specificities for a binding partner and an increased valency. This has important advantages, as set out below. The soluble proteins are for use as therapeutics.

The present invention further provides improved soluble SIRPα binding proteins for use as therapeutics. SIRPα-binding antibody-like proteins as defined in the present invention may provide means to increase avidity to targeted SIRPα expressing cells compared to prior art CD47 protein fusions, while maintaining excellent developability properties. Additionally, without being bound by any theory, a higher avidity is expected to result in longer pharmaco-dynamic half-life thus providing enhanced therapeutic efficacy. These new findings offer new therapeutic tools to target SIRPα expressing cells and represent therapeutic perspectives, in particular for multiple autoimmune and inflammatory disorders, cancer disorders or stem cell transplantation.

Therefore, in one aspect, the invention provides a soluble protein, comprising a complex of two heterodimers, wherein each heterodimer essentially consists of:

(i) a first single chain polypeptide comprising:
(a) an antibody heavy chain sequence having VH, CH1, CH2, and CH3 regions; and
(b) a monovalent region of a mammalian binding molecule fused to the VH region; and
   (ii) a second single chain polypeptide comprising:
(c) an antibody light chain sequence having a VL and CL region; and
(d) a monovalent region of a mammalian binding molecule fused to the VL region;
   characterised in that each pair of VH and VL CDR sequences has specificity for an antigen, such that the total valency of said soluble protein is six.

The applicant has previously developed antibody-like molecules, termed “Fusobodies” wherein the variable regions of both arms of an antibody are replaced by regions of a mammalian binding molecule, for example SIRPα binding domains, thereby providing a multivalent soluble protein. The soluble proteins of the present invention are similar to the applicant's Fusobodies in that these molecules also comprise antibody sequences. However with the molecules of the present invention, the VH and VL regions of the antibody sequence—and the associated valency and antigen specificity—have been retained, these regions being fused to regions of a mammalian binding molecule. The molecules of the present invention thus have one or more binding specificities provided by the bivalent antibody sequences, and further specificities provided by the four monovalent regions of a mammalian binding molecule. To differentiate the soluble proteins of the present invention from those previously developed by the applicant, the term “Extended Fusobody” will be used hereinafter. The applicant's previously developed molecules will continue to be referred to as “Fusobodies”, or “non-extended Fusobodies”.

One example of an Extended Fusobody is shown in FIG. 1, which also depicts the applicant's previously developed Fusobody, together with a reference CD47-Fc molecule.

Compared to prior art molecules, the soluble proteins of the invention have increased valency. The heterodimers of the invention preferably have a valency of three, based on monovalency per polypeptide chain and each pair of VH and VL regions further providing a monovalent antigen binding specificity. The soluble proteins of the invention therefore have a valency of six (hexavalency), based on tetravalency contributed by the regions of the mammalian binding molecule on the four polypeptide chains, and a bivalency contributed by the antibody VH and VL regions. In preferred embodiments, each single chain polypeptide is monovalent, each heterodimer is trivalent, and each soluble protein (based on a complex of two heterodimers) is hexavalent. By incorporation of a monovalent binding molecule in each first and second single chain polypeptide, and a monovalent antigen binding specificity provided by each pair of VH and VL regions, the valency of each heterodimer is three, i.e. each heterodimer can bind up to three separate binding partners, or up to three times on the same binding partner. This is to be contrasted with prior art molecules (for example those disclosed in WO 01/46261) where the valency of a heterodimer of first and second polypeptide chains is one (i.e. both chains are required to bind the binding partner), to the extent that a complex of two heterodimers has a valency of two. A complex of two trivalent heterodimers of the invention has a valency of six, i.e. the protein can bind up to six binding partners, or up to six times on the same binding partner. The heterodimers of the invention are trivalent and a complex of heterodimers has a valency of n×3, where n is the number of heterodimers comprised within the complex. In preferred embodiments, the complex comprises two heterodimers, and has a valency of 6. Complexes comprising more than two heterodimers have a valency greater than 6, for example 9, 12, 15 or 18. The increased valency of the soluble proteins of the invention results in a higher avidity, with advantageous effects on half-life and efficacy. Beyond these effects another advantage of a therapeutic molecule having high-avidity (compared to one having lower avidity) is that a reduction in dosing can be used, for example by up to a factor of ten.

An antibody-like molecule having dual-variable domains fused to the constant region of an antibody is disclosed in WO 2010/127284. The disclosed molecules are bispecific and have a valency of four, this being derived from the two pairs of VH and VL regions on each arm of the molecule. One of the key differences between the soluble proteins or Extended Fusobodies of the present invention and the dual-variable domain molecules disclosed in WO 2010/127284 is that only one variable domain (i.e. VH and VL) is employed on each arm of the soluble protein/Extended Fusobody of the invention. By using monovalent regions of a mammalian binding molecule—for example an extracellular domain of a cell surface receptor such as CD47—instead of a second variable domain, specificity for a second (or third antigen) can be still obtained. One of the advantages of using a natural receptor domain is that the interaction with its cognate binding partner is more predictable, natural, specific, and in a therapeutic context, the domains of the mammalian binding molecule have no expected immunogenicity, compared to a therapeutic antibody or dual-variable domain molecule, which may comprise immunogenic regions and/or mutations to improve specificity, affinity and avidity. Compared to dual-variable domain molecules, another advantage in using monovalent mammalian binding regions fused to an antibody variable domain is that the problem of conformationally positioning the regions of the mammalian binding molecule next to the antibody variable domain (and yet retaining the required binding specificities) is far simpler than positioning two variable domains with different specificities, where precise and optimal use of linkers is invariably required. Thus, the multiple specificities achieved with prior art molecules can be achieved more easily with the soluble proteins of the invention, and whereby the molecules provide an increased valency and further advantages.

In one aspect the invention provides a multivalent soluble protein complex comprising two or more soluble proteins of the invention, wherein if the protein complex comprises N soluble proteins, the valency is N×6.

Therefore, in one aspect, the invention provides a soluble protein having at least hexavalency (or being at least hexavalent), comprising a complex of at least two heterodimers, wherein each heterodimer essentially consists of:

(i) a first single chain polypeptide comprising:
(a) an antibody heavy chain sequence having VH, CH1, CH2, and CH3 regions; and
(b) a region of a mammalian binding molecule fused to the VH region; and
   (ii) a second single chain polypeptide comprising:
(c) an antibody light chain sequence having a VL and CL region; and
(d) a region of a mammalian binding molecule fused to the VL region; characterised in that each pair of VH and VL CDR sequences has specificity for an antigen (i.e. is monovalent), and each region of a mammalian binding molecule has monovalency such that the total valency of said soluble protein is six.

In another aspect, the invention provides a complex of soluble proteins, each soluble protein, having at least hexavalency (or being at least hexavalent), comprising a complex of at least two heterodimers, wherein each heterodimer essentially consists of:

(i) a first single chain polypeptide comprising:
(a) an antibody heavy chain sequence having VH, CH1, CH2, and CH3 regions; and
(b) a region of a mammalian binding molecule fused to the VH region; and
   (ii) a second single chain polypeptide comprising:
(c) an antibody light chain sequence having a VL and CL region; and
(d) a region of a mammalian binding molecule fused to the VL region; characterised in that each pair of VH and VL CDR sequences has specificity for an antigen (i.e. is monovalent), and each region of a mammalian binding molecule has monovalency such that the total valency of said soluble protein is six, and wherein if the protein complex comprises N soluble proteins, the valency is N×6.

In another aspect the invention provides a soluble protein, comprising a complex of two heterodimers, wherein each heterodimer essentially consists of:

(i) a first single chain polypeptide comprising:
(a) a modified antibody heavy chain sequence having two CH1 regions, CH2, and CH3 regions in the order CH1-CH1-CH2-CH3; and
(b) a monovalent region of a mammalian binding molecule fused to the first CH1 region; and
   (ii) a second single chain polypeptide comprising:
(c) a modified antibody light chain sequence having two fused CL regions; and
(d) a monovalent region of a mammalian binding molecule fused to the first VL region; characterised in that the total valency of said soluble protein is four.

In this aspect the valency of the soluble protein is four, however, the molecule retains an Extended Fusobody-like structure because the VH and VL sequences are replaced with CH₁ and CL sequences, respectively.

In another aspect the invention provides a soluble protein, comprising a complex of two heterodimers, wherein each heterodimer comprises:

(i) a first single chain polypeptide comprising:
(a) an antibody heavy chain sequence having VH, CH1, CH2, and CH3 regions; and
(b) at least one monovalent region of a mammalian binding molecule fused to the VH region; and
   (ii) a second single chain polypeptide comprising:
(c) an antibody light chain sequence having a VL and CL region; and
(d) at least one monovalent region of a mammalian binding molecule fused to the VL region; characterised in that each pair of VH and VL CDR sequences has specificity for an antigen, such that the total valency of said soluble protein is at least six.

In a preferred aspect the soluble protein has binding specificity for one, two or three antigens. The binding specificity arises from (i) the antigen binding specificity of the VH and VL regions of the antibody sequence, and (ii) the binding specificity of each region of the mammalian binding molecule.

In a preferred aspect the VH and VL regions within each heterodimer are specific for the same antigen, preferably the same epitope on that antigen.

In a preferred aspect the mammalian binding molecule comprised within said first and second single chain polypeptides is the same. In a more preferred aspect the regions of the mammalian binding molecule comprised within said first and second single chain polypeptides are the same.

Therefore, the invention provides a soluble protein, comprising a complex of two heterodimers, wherein each heterodimer essentially consists of:

(i) a first single chain polypeptide comprising:
(a) an antibody heavy chain sequence having VH, CH1, CH2, and CH3 regions; and
(b) a monovalent region of a mammalian binding molecule fused to the VH region; and
   (ii) a second single chain polypeptide comprising:
(c) an antibody light chain sequence having a VL and CL region; and
(d) a monovalent region of the same mammalian binding molecule fused to the VL region; characterised in that each pair of VH and VL CDR sequences has specificity for an antigen, such that the total valency of said soluble protein is six.

The invention further provides a soluble protein, comprising a complex of two heterodimers, wherein each heterodimer essentially consists of:

(i) a first single chain polypeptide comprising:
(a) an antibody heavy chain sequence having VH, CH1, CH2, and CH3 regions; and
(b) a monovalent region of a mammalian binding molecule fused to the VH region; and
   (ii) a second single chain polypeptide comprising:
(c) an antibody light chain sequence having a VL and CL region; and
(d) the same region of the same mammalian binding molecule fused to the VL region; characterised in that each pair of VH and VL CDR sequences has specificity for an antigen, such that the total valency of said soluble protein is six.

In one embodiment each region of the mammalian binding molecule and each pair of VH and VL CDR sequences has binding specificity for the same single antigen. In one embodiment, the regions of the mammalian binding molecule can bind a first epitope on the antigen, and each pair of VH and VL CDR sequences can bind a second epitope on the same antigen. In another embodiment, the regions of the mammalian binding molecule and each pair of VH and VL CDR sequences can bind the same epitope on the same antigen.

In one embodiment the soluble protein or Extended Fusobody of the invention has binding specificity for two antigens, wherein each region of the mammalian binding molecule has binding specificity for a first antigen, and each pair of VH and VL CDR sequences has binding specificity for a second antigen. In a specific embodiment, a SIRPα-binding protein of the invention has specificity for SIRPα (based on an extracellular binding domain of CD47 comprised within each polypeptide sequence) and either TNF alpha or cyclosporin A, based on the specifity of the VH/VL and associated CDR sequences.

In another embodiment, the mammalian binding molecule comprised within said first and second single chain polypeptides is different. Therefore the invention provides a soluble protein, comprising a complex of two heterodimers, wherein each heterodimer essentially consists of:

(i) a first single chain polypeptide comprising:
(a) an antibody heavy chain sequence having VH, CH1, CH2, and CH3 regions; and
(b) a monovalent region of a first mammalian binding molecule fused to the VH region; and
   (ii) a second single chain polypeptide comprising:
(c) an antibody light chain sequence having a VL and CL region; and
(d) a monovalent region of a second mammalian binding molecule fused to the VL region; characterised in that said first and second mammalian binding molecules have binding specificities for first and second antigens, and each pair of VH and VL CDR sequences has specificity for either said first or said second antigen, whereby the soluble protein is bispecific, having a total valency of is six.

In an alternative embodiment, the VH and VL regions may bind a different antigen to the one or two antigens bound by the regions of the mammalian binding molecule. Such an Extended Fusobody is trispecific, i.e. can bind three different antigens, wherein the regions of the mammalian binding molecule comprised within the first single polypeptide chain have binding specificity for a first antigen, the regions of the mammalian binding molecule comprised within the second single polypeptide chain have binding specificity for a second antigen, and each pair of VH and VL CDR sequences has binding specificity for a third antigen. Therefore, the invention provides a soluble protein, comprising a complex of two heterodimers, wherein each heterodimer essentially consists of:

(i) a first single chain polypeptide comprising:
(a) an antibody heavy chain sequence having VH, CH1, CH2, and CH3 regions; and
(b) a monovalent region of a first mammalian binding molecule fused to the VH region; and
   (ii) a second single chain polypeptide comprising:
(c) an antibody light chain sequence having a VL and CL region; and
(d) a monovalent region of a second mammalian binding molecule fused to the VL region; characterised in that said first and second mammalian binding molecules have binding specificities for first and second antigens, and each pair of VH and VL CDR sequences has specificity for a third second antigen, whereby the soluble protein is trspecific, having a total valency of six.

In specific embodiments, the VH and VL CDR sequences have binding specificity for TNFalpha, or cyclosporin A, or epitopes derived therefrom.

In a preferred embodiment, the region of a mammalian binding molecule is fused to the N-terminal part of the antibody sequence (i.e. to the VH and VL constant regions). Thus, the C-terminus of the region of the mammalian binding molecule is fused to the N-terminus of the antibody sequence. In some embodiments the sequences are joined directly, in some embodiments a linker sequence can be used.

In one embodiment the binding molecule is a cytokine, growth factor, hormone, signaling protein, low molecular weight compound (drug), ligand, or cell surface receptor. Preferably, the binding molecule is a mammalian monomeric or homo-polymeric cell surface receptor. The region of the binding molecule may be the whole molecule, or a portion or fragment thereof, which may retain its biological activity. The region of the binding molecule may be an extracellular region or domain. In one embodiment, said mammalian monomeric or homo-polymeric cell surface receptor comprises an immunoglobulin superfamily (IgSF) domain, for example it comprises a SIRPalpha binding domain, which may be the extracellular domain of CD47.

In one embodiment, the invention relates to isolated soluble SIRPα-binding proteins or SIRPα-binding Extended Fusobodies, comprising a hexavalent complex of two trivalent heterodimers, wherein each heterodimer essentially consists of:

(i) a first single chain polypeptide comprising a first SIRPα-binding domain fused at the N-terminal part of a VH region of an antibody; and,
(ii) a second single chain polypeptide comprising a second SIRPα-binding domain fused at the N-terminal part of VL region of an antibody.

In a preferred embodiment, the C_{H}1, C_{H}2 and C_{H}3 regions can be derived from wild type or mutant variants of human IgG1, IgG2, IgG3 or IgG4 corresponding regions with silent effector functions and/or reduced cell killing, ADCC or CDC effector functions, for example reduced ADCC effector functions.

In one embodiment, said soluble protein or SIRPα-binding Extended Fusobody dissociates from binding to human SIRPα with a k_{off} (kd1) of 0.05 [1/s] or less, as measured by surface plasmon resonance, such as a BiaCORE assay, applying a bivalent kinetic fitting model.

In another embodiment, said soluble protein or SIRPα binding Fusobody inhibits the Staphylococcus aureus Cowan strain particles stimulated release of proinflammatory cytokines in in vitro generated monocyte-derived dendritic cells.

For example, said soluble protein or SIRPα binding Fusobody inhibits the Staphylococcus aureus Cowan strain particles stimulated release of proinflammatory cytokines in in vitro generated monocyte-derived dendritic cells, with an IC₅₀ of 2 nM or less, 1 nM or less, 0.2 nM or less, 0.1 nM or less, for example between 10 pM and 2 nM, or 20 pM and 1 nM, or 30 pM and 0.2 nM, as measured in a dendritic cell cytokine release assay.

In another related embodiment, said first and second single chain polypeptides of each heterodimer are covalently bound by a disulfide bridge, for example using a natural disulfide bridge between cysteine residues of the corresponding C_{H}1 and C_{L} regions.

In one embodiment, each region of said mammalian binding molecule is fused to its respective VH or VL sequence in the absence of a peptide linker. In another embodiment, each region of said mammalian binding molecule is fused to its respective VH or VL sequence via a peptide linker. The peptide linker may comprise 5 to 20 amino acids, for example, it may be a polymer of glycine and serine amino acids, preferably of (GGGGS)ₙ, wherein n is any integer between 1 and 4, preferably 2.

In one preferred embodiment, said soluble protein or SIRPα binding Extended Fusobody essentially consists of two heterodimers, wherein said first single chain polypeptide of each heterodimer comprises the hinge region of an immunoglobulin constant part, and the two heterodimers are stably associated with each other by a disulfide bridge between the cysteines at their hinge regions.

In one embodiment, the soluble protein of the invention comprises at least one SIRPα binding domain selected from the group consisting of:
(i) an extracellular domain of the human cell surface receptor CD47;
(ii) an extracellular domain derived from SEQ ID NO:2;
(iii) a polypeptide of SEQ ID NO:3, SEQ ID NO:4, SEQ ID NO:5, SEQ ID NO:57 or a fragment thereof retaining SIRPα binding properties; and,
(iv) a variant polypeptide of SEQ ID NO:3, SEQ ID NO:4, SEQ ID NO:5, SEQ ID NO:57 or said fragment, having at least 60, 70, 80, 90, 95, 96, 97, 98, or 99 percent sequence identity, and retaining SIRPα binding properties.

In a preferred embodiment, the region of an extracellular domain of CD47 is SEQ ID NO:3, SEQ ID NO:4 or SEQ ID NO:57.

In one specific embodiment, two or more SIRPα binding domains comprised within said first and second single polypeptide chains share at least 60, 70, 80, 90, 95, 96, 97, 98, 99, or 99.5% percent sequence identity with each other. In a preferred embodiment, two or more SIRPα binding domains have identical amino acid sequences.

In one specific embodiment, all SIRPα binding domains within the SIRPα binding Extended Fusobody have identical amino acid sequences. For example, all SIRPα binding domains consist of SEQ ID NO:3 or SEQ ID NO:4 or SEQ ID NO:5 or SEQ ID NO:57.

In one specific embodiment, said soluble protein of the invention or SIRPα binding Extended Fusobody comprises two heterodimers, wherein each heterodimer essentially consists of:

(i) a first single heavy chain polypeptide of SEQ ID NO:20 and a second single light chain polypeptide of SEQ ID NO:21;
(ii) a first single heavy chain polypeptide of SEQ ID NO:22 and a second single light chain polypeptide of SEQ ID NO:23; or
(ii) a first single heavy chain polypeptide of SEQ ID NO:40 and a second single light chain polypeptide of SEQ ID NO:41.

Said first and second single chain polypeptides are stably associated at least via one disulfide bond, similar to the heavy and light chains of an antibody.

In a related embodiment, the soluble protein or SIRPα binding Fusobody comprises two heterodimers, wherein the first and second single chain polypeptides of each heterodimer have at least 60, 70, 80, 90, 95, 96, 97, 98, or 99 percent sequence identity to corresponding first and second single chain polypeptide of (i) SEQ ID NO:20 and SEQ ID NO:21; (ii) SEQ ID NO:22 and SEQ ID NO:23; or (ii) SEQ ID NO:40 and SEQ ID NO:41 respectively. Preferably, these molecules retain the advantageous functional properties of a SIRPα binding Extended Fusobody as described above.

In one specific embodiment, the four SIRPα binding domains of a SIRPα binding Extended Fusobody according to the invention are identical in sequence.

The invention further relates to such multivalent soluble protein complexes comprising two or more Extended Fusobodies or SIRPα-binding Extended Fusobodies, wherein if the protein complex comprises N soluble proteins, the valency is N×6.

The invention further relates to such soluble proteins or Extended Fusobodies, in particular SIRPα-binding proteins or Extended Fusobodies for use as a drug or diagnostic tool, for example in the treatment or diagnosis of autoimmune and acute and chronic inflammatory disorders. In particular SIRPα-binding proteins or Extended Fusobodies are for use in a treatment selected from the group consisting of Th2-mediated airway inflammation, allergic disorders, asthma, inflammatory bowel diseases and arthritis.

The soluble proteins or Fusobodies of the invention may also be used in the treatment or diagnosis of ischemic disorders, leukemia or other cancer disorders, or in increasing hematopoietic stem engraftment in a subject in need thereof.

### DEFINITIONS

In order that the present invention may be more readily understood, certain terms are first defined. Additional definitions are set forth throughout the detailed description.

The term SIRPα refers to the human Signal Regulatory Protein Alpha (also designated CD172a or SHPS-1) which shows adhesion to CD47 (Integrin associated protein). Human SIRPα includes SEQ ID NO:1 but further includes, without limitation, any natural polymorphic variant, for example, comprising single nucleotide polymorphisms (SNPs), or splice variants of human SIRPα. Examples of splice variants or SNPs in SIRPα nucleotide sequence found in human are described in Table 1.

TABLE 1
Variants of SIRPα Protein

| Variant | | |
|---|---|---|
| Type | Variant ID | Description |
| Splice | NP_542970.1 | reference; short variant; |
| Variant | ENSP00000382941 | sequence NO: 1 |
| | | long variant, insertion of |
| | | four amino acids close to |
| | | C-terminus |
| Single | rs17855609 | DNA: A or T; protein: T or S |
| Nucleotide | | (pos. 50 of NP_542970.1) |
| Polymorphism | rs17855610 | DNA: C or T; protein: T or I |
| | | (pos. 52 of NP_542970.1) |
| | rs17855611 | DNA: G or A; protein: R or H |
| | | (pos. 54 of NP_542970.1) |
| | rs17855612 | DNA: C or T; protein: A or V |
| | | (pos. 57 of NP_542970.1) |
| | rs1057114 | DNA: G or C; protein: G or A |
| | | (pos. 75 of NP_542970.1) |
| | rs1135200 | DNA: C or G; protein: D or E |
| | | (pos. 95 of NP_542970.1) |
| | rs17855613 | DNA: A or G; protein: N or D |
| | | (pos. 100 of NP_542970.1) |
| | rs17855614 | DNA: C or A; protein: N or K |
| | | (pos. 100 of NP_542970.1) |
| | rs17855615 | DNA: C or A; protein: R or S |
| | | (pos. 107 of NP_542970.1) |
| | rs1135202 | DNA: G or A; protein: G or S |
| | | (pos. 109 of NP_542970.1) |
| | rs17855616 | DNA: G or A; protein: G or S |
| | | (pos. 109 of NP_542970.1) |
| | rs2422666 | DNA: G or C; protein: V or L |
| | | (pos. 302 of NP_542970.1) |
| | rs12624995 | DNA: T or G; protein: V or G |
| | | (pos. 379 of NP_542970.1) |
| | rs41278990 | DNA: C or T; protein: P or S |
| | | (pos. 482 of NP_542970.1) |

The term CD47 refers to Integrin associated protein, a mammalian membrane protein involved in the increase in intracellular calcium concentration that occurs upon cell adhesion to extracellular matrix. Human CD47 includes SEQ ID NO:2 but also any natural polymorphic variant, for example, comprising single nucleotide polymorphisms (SNPs), or splice variants of human CD47. Examples of splice variants or SNPs in CD47 nucleotide sequence found in human are described in Table 2.

TABLE 2
Variants of CD47 Protein

| Variant | | |
|---|---|---|
| Type | Variant ID | Description |
| Splice | NP_001768.1 | reference; longest variant; |
| Variant | | sequence NO: 2 |
| | NP_942088.1 | different, shorter C-terminus |
| | NP_001020250.1 | different, shorter C-terminus |
| | ENSP00000381308 | different, shorter C-terminus |
| Single | rs11546646 | DNA: C or G; protein: A or P |
| Nucleotide | | (pos. 96 of NP_001768.1) |
| Polymorphism | ENSSNP12389584 | DNA: C or G; protein: V or L |
| | | (pos. 246 of NP_001768.1) |

As used herein, the term “protein” refers to any organic compounds made of amino acids arranged in one or more linear chains and folded into a globular form. The amino acids in a polymer chain are joined together by the peptide bonds between the carboxyl and amino groups of adjacent amino acid residues. The term “protein” further includes, without limitation, peptides, single chain polypeptide or any complex molecules consisting primarily of two or more chains of amino acids. It further includes, without limitation, glycoproteins or other known post-translational modifications. It further includes known natural or artificial chemical modifications of natural proteins, such as without limitation, glycoengineering, pegylation, hesylation and the like, incorporation of non-natural amino acids, and amino acid modification for chemical conjugation with another molecule.

As used herein, a “complex protein” refers to a protein which is made of at least two single chain polypeptides, wherein said at least two single chain polypeptides are associated together under appropriate conditions via either non-covalent binding or covalent binding, for example, by disulfide bridge. A “heterodimeric protein” refers to a protein that is made of two single chain polypeptides forming a complex protein, wherein said two single chain polypeptides have different amino acid sequences, in particular, their amino acid sequences share not more than 90, 80, 70, 60 or 50% identity between each other. To the contrary, a “homodimeric protein” refers to a protein that is made of two identical or substantially identical polypeptides forming a complex protein, wherein said two single chain polypeptides share 100% identity, or at least 99% identity, or at least 95%, the amino acid differences consisting of amino acid substitution, addition or deletion which does not affect the functional and physical properties of the polypeptide compared to the other one of the homodimer, for example conservative amino acid substitutions.

As used herein, a protein is “soluble” when it lacks any transmembrane domain or protein domain that anchors or integrates the polypeptide into the membrane of a cell expressing such polypeptide. In particular, the soluble proteins of the invention may likewise exclude transmembrane and intracellular domains of CD47. As used herein the term “antibody” refers to a protein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, C_{H}1, C_{H}2 and C_{H}3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g. effector cells) and the first component (C1q) of the classical complement system.

The terms “complementarity determining region,” and “CDR,” refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The amino acid sequence boundaries of a given CDR can be determined by a number of methods, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme). The phrase “constant region” refers to the portion of the antibody molecule that confers effector functions.

As used in the present text, the term “Fusobody” (or “non-extended Fusobody”) refers to an antibody-like soluble protein comprising two heterodimers, each heterodimer consisting of one heavy and one light chain of amino acids, stably associated together, for example via one or more disulfide bond(s). Each heavy or light chain comprises constant regions of an antibody, referred hereafter respectively as the heavy and light chain constant regions of the Fusobody. The heavy chain constant region comprises at least the C_{H}1 region of an antibody and may further comprise C_{H}2 and C_{H}3 regions, including the hinge region. The light chain constant region comprises the C_{L} region of an antibody. In a Fusobody, the variable regions of an antibody are replaced by regions of a mammalian binding molecule, these being heterologous soluble binding domains. The term “heterologous” means that these domains are not naturally found associated with constant regions of an antibody. In particular, such heterologous binding domains do not have the typical structure of an antibody variable domain consisting of 4 framework regions, FR1, FR2, FR3 and FR4 and the 3 complementarity determining regions (CDRs) in-between. Each arm of the Fusobody therefore comprises a first single chain polypeptide comprising a first binding domain covalently linked at the N-terminal part of a constant C_{H}1 heavy chain region of an antibody, and a second single chain polypeptide comprising a second binding domain covalently linked at the N-terminal part of a constant C_{L} light chain region of an antibody. The covalent linkage may be direct, for example via peptidic bound or indirect, via a linker, for example a peptidic linker. The two heterodimers of the Fusobody are covalently linked, for example, by at least one disulfide bridge at their hinge region, like an antibody structure.

“Extended Fusobody” refers to an antibody-like soluble protein comprising two heterodimers, each heterodimer consisting of one heavy and one light chain of amino acids, stably associated together, for example via one or more disulfide bond(s). Each heavy or light chain comprises the constant and variable regions of an antibody, referred hereafter respectively as the heavy and light chain regions of the Extended Fusobody. Within the heavy chain the constant region comprises the C_{H}1, C_{H}2 and C_{H}3 regions of an antibody, including the hinge region. The C_{H}2 and C_{H}3 regions of an antibody, are referred to as the Fc part or Fc moiety of the Extended Fusobody, by analogy to antibody structure. Detailed description of the Fc part of an Extended Fusobody is described in a paragraph further below. Within the light chain the light chain constant region comprises the C_{L} region of an antibody. Fused to the VH and VL regions are regions of a mammalian binding molecule, these being heterologous soluble binding domains. The term “heterologous” means that these domains are not naturally found associated with the variable or constant regions of an antibody and do not have the typical structure of an antibody variable domain consisting of 4 framework regions, FR1, FR2, FR3 and FR4 and the 3 CDRs in-between. Each arm of the Extended Fusobody therefore comprises a first single chain polypeptide comprising a first binding domain covalently linked at the N-terminal part of a VH region of a heavy chain of an antibody, and a second single chain polypeptide comprising a second binding domain covalently linked at the N-terminal part of a VL region of a light chain of an antibody. The covalent linkage may be direct, for example via peptidic bond or indirect, via a linker, for example a peptidic linker. The two heterodimers of the Extended Fusobody are covalently linked, for example, by at least one disulfide bridge at their hinge region, like an antibody structure. As described previously, an Extended Fusobody has specificity for an antigen provided by its VH and VL regions, and further specificities provided by the heterologous soluble binding domains fused to the antibody heavy and light chain sequences.

As used herein, the term “Fc region” is used to define the C-terminal region of an immunoglobulin heavy chain and the soluble proteins and Extended Fusobodies of the invention. The definition includes native sequence Fc region and variant Fc regions. The human IgG heavy chain Fc region is generally defined as comprising the amino acid residue from position C226 or from P230 to the carboxyl-terminus of the IgG antibody. The numbering of residues in the Fc region is that of the EU index of Kabat. The C-terminal lysine (residue K447) of the Fc region may be removed, for example, during production or purification of the antibody.

The term “valency” of an antibody refers to the number of antigenic determinants that an individual antibody molecule can bind. The valency of all antibodies is at least two and in some instances more.

The term “avidity” is used to describe the combined strength of multiple bond interactions between proteins. Avidity is distinct from affinity which describes the strength of a single bond. As such, avidity is the combined synergistic strength of bond affinities (functional affinity) rather than the sum of bonds. With the Extended Fusobodies of the invention, the regions of the mammalian binding molecule and the antigen binding sites from the VH/VL pairs simultaneously interact with their respective binding partners. Whilst each single binding interaction may be readily broken (depending on the relative affinity), because many binding interactions are present at the same time, transient unbinding of a single site does not allow the molecule to diffuse away, and binding of that site is likely to be reinstated. The overall effect is synergistic, strong binding of antigen to antibody (e.g. IgM is said to have low affinity but high avidity because it has 10 weak binding sites as opposed to the 2 strong binding sites of IgG, IgE and IgD). FIG. 1 is a schematic representation of a Fusobody and Extended Fusobody molecule, compared with a reference CD47-Fc molecule. Examples of molecules with a Fusobody-like structure have been described in the art, in particular, molecules comprising ligand binding regions of a heterodimeric receptor where both chains of each heterodimer are required to bind each ligand i.e. having a valency of one per heterodimer, and a total valency of two for a protein consisting of two heterodimers, (see for example WO 01/46261).

In a preferred embodiment, the extracellular domain of a mammalian monomeric or homopolymeric cell surface receptor or a variant or region of such extracellular domain retaining ligand binding activities, is fused to the variable regions of the heavy and light chains of an antibody. The resulting Extended Fusobody molecule is a multivalent protein retaining the advantageous properties of an antibody molecule for use as a therapeutic molecule.

The term “mammalian binding molecule” as used herein is any molecule, or portion or fragment thereof, that can bind to a target molecule, cell, complex and/or tissue, and which includes proteins, nucleic acids, carbohydrates, lipids, low molecular weight compounds, and fragments thereof, each having the ability to bind to one or more of members selected from the group consisting of: soluble protein, cell surface protein, cell surface receptor protein, intracellular protein, carbohydrate, nucleic acid, a hormone, or a low molecular weight compound (small molecule drug), or a fragment thereof. The mammalian binding molecule may be a protein, cytokine, growth factor, hormone, signaling protein, inflammatory mediator, ligand, receptor, or fragment thereof. In preferred embodiments, the mammalian binding molecule is a native or mutated protein belonging to the immunoglobulin superfamily; a native hormone or a variant thereof being able to bind to its natural receptor; a nucleic acid or polynucleotide sequence being able to bind to complementary sequence and/or soluble cell surface or intracellular nucleic acid/polynucleotide binding proteins; a carbohydrate binding moiety being able to bind to other carbohydrate binding moieties and/or soluble, cell surface or intracellular proteins; a low molecular weight compound (drug) that binds to a soluble or cell surface or intracellular target protein.

The term “IgSF-domains” refers to the immunoglobulin super-family domain containing proteins comprising a vast group of cell surface and soluble proteins that are involved in the immune system by mediating binding, recognition or adhesion processes of cells. The immunoglobulin domain of the IgSF-domain molecules share structural similarity to immunoglobulins. IgSF-domains contain about 70-110 amino acids and are categorized according to their size and function. Ig-domains possess a characteristic Ig-fold, which has a sandwich-like structure formed by two sheets of antiparallel beta strands. The Ig-fold is stabilized by a highly conserved disulfide bonds formed between cysteine residues as well as interactions between hydrophobic amino acids on the inner side of the sandwich. One end of the Ig domain has a section called the complementarity determining region that is important for the specificity of the IgSF domain. Most Ig domains are either variable (IgV) or constant (IgC). Examples of proteins displaying one or more IgSF domains are cell surface co-stimulatory molecules (CD28, CD80, CD86), antigen receptors (TCR/BCR) co-receptors (CD3/CD4/CD8). Other examples are molecules involved in cell adhesion (ICAM-1, VCAM-1) or with IgSF domains forming a cytokine binding receptor (IL1R, IL6R) as well as intracellular muscle proteins. In many examples, the presence of multiple IgSF domains in close proximity to the cellular environment is a requirement for efficacy of the signaling triggered by said cell surface receptor containing such IgSF domain. A prominent example is the clustering of IgSF domain containing molecules (CD28, ICAM-1, CD80 and CD86) in the immunologic synapse that enables a microenvironment allowing optimal antigen-presentation by antigen-presenting cells as well as resulting in controlled activation of naive
cells (Dustin, 2009, Immunity). Other examples for other IgSF containing molecules that need clustering for proper function are CD2 (Li, et al. 1996, J. Mol. Biol., 263(2):209-26) and ICAM-1 (Jun, et al. 2001, J. Biol. Chem.; 276(31):29019-27).

Therefore, by mimicking an oligovalent structure containing IgSF domain, the Extended Fusobodies of the invention comprising several IgSF domains may advantageously be used for modulating the activity of their corresponding binding partner.

As used herein, the term SIRPγ refers to CD172g. Human SIRPγ includes SEQ ID NO:115 but also any natural polymorphic variant, for example, comprising single nucleotide polymorphisms (SNPs), or splice variants of human SIRPγ. Examples of splice variants or SNPs in SIRPγ nucleotide sequence found in human are described in Table 3.

TABLE 3
Variants of SIRPγ Protein

| Variant | | |
|---|---|---|
| Type | Variant ID | Description |
| Splice | NP_061026.2 | SEQ ID NO: 115 |
| Variant | NP_001034597.1 | aas 250-360 missing |
| | NP_543006 | aas 144-360 missing |
| | ENSP00000370992 | aas 1-33 missing |
| Single | rs6074959 | DNA: G or T; protein: A or S |
| Nucleotide | | (pos. 5 of NP_061026.2) |
| Polymorphism | rs6043409 | DNA: T or C; protein: V or A |
| | | (pos. 263 of NP_061026.2) |
| | rs6034239 | DNA: C or T; protein: S or L |
| | | (pos. 286 of NP_061026.2) |
| | rs41275436 | DNA: G or C; protein: V or L |
| | | (pos. 316 of NP_061026.2) |
| | rs41275434 | DNA: C or T; protein: A or V |
| | | (pos. 338 of NP_061026.2) |
| | rs35062363 | DNA: C or T; protein: A or V |
| | | (pos. 368 of NP_061026.2) |

The term “bivalent kinetic fitting model” as used herein refers to a model which describes the binding of a bivalent analyte to a monovalent ligand as described in Baumann et al., (1998, J. Immunol. Methods, 221(1-2):95-106), the contents of which are incorporated by reference. In this model two sets of rate constants are generated, one rate constant for each binding step, ka1, ka2, kd1 and kd2. The term “k_{assoc}” or “kₐ”, as used herein, is intended to refer to the association rate constant of a particular protein-protein interaction, whereas the term “k_{dis}” or “k_{d}” as used herein, is intended to refer to the dissociation rate constant of a particular protein-protein interaction. The term “k_{off}” is used as a synonym for k_{dis} or kd1 or the dissociation rate constant. The term “K_{D}”, as used herein, is intended to refer to the dissociation constant, which is obtained from the ratio of k_{d} to kₐ (i.e. k_{d}/kₐ) and is expressed as a molar concentration (M) for K_{D1} and as resonance units (RU) for K_{D2}. K_{D2} (RU) can be converted to a molar concentration (M) as described in Baumann et al. K_{D} values for protein-protein interactions can be determined using methods well established in the art. For example, a method for determining the K_{D} (or K_{D1} or K_{D2}) of a protein/protein interaction is by using surface plasmon resonance, or using a biosensor system such as a BiaCORE system. At least one assay for determining the K_{D} values of the proteins of the invention interacting with SIRPα is described in the Examples below.

As used herein, the term “affinity” refers to the strength of interaction between the polypeptide and its target at a single site. Within each site, the binding region of the polypeptide interacts through weak non-covalent forces with its target at numerous sites; the more interactions, the stronger the affinity.

As used herein, the term “high affinity” for a binding polypeptide or protein refers to a polypeptide or protein having a K_{D} of 1 μM or less for its target.

In one embodiment, the soluble protein of the invention inhibits immune complex-stimulated cell cytokine (e.g. IL-6, IL-10, IL-12p70, IL-23, IL-8 and/or TNF-α) release from peripheral blood monocytes, conventional dendritic cells (DCs) and/or monocyte-derived DCs stimulated with Staphylococcus aureus Cowan 1 (Pansorbin) or soluble CD40L and IFN-γ. One example of an immune complex-stimulated dendritic cell cytokine release assay is the Staphylococcus aureus Cowan strain particles stimulated release of proinflammatory cytokines in in vitro generated monocyte-derived dendritic cells described in more details in the Examples below. In a preferred embodiment, a protein that inhibits immune complex-stimulated cell cytokine release is a protein that inhibits the Staphylococcus aureus Cowan strain particles stimulated release of proinflammatory cytokines in of in vitro generated monocyte-derived dendritic cells with an IC₅₀ of 2 nM or less, 0.2 nM or less, 0.1 nM or less for example between 2 nM and 20 pM, or 1 nM and 10 pM as measured in a dendritic cell cytokine release assay.

As used herein, unless otherwise defined more specifically, the term “inhibition”, when related to a functional assay, refers to any statistically significant inhibition of a measured function when compared to a negative control.

Assays to evaluate the effects of the soluble proteins or Extended Fusobodies of the invention on functional properties of SIRPα are described in further detail in the Examples.

As used herein, the term “subject” includes any human or non-human animal.

The term “non-human animal” includes all vertebrates, e.g. mammals and non-mammals, such as non-human primates, sheep, dogs, cats, horses, cows, chickens, amphibians, reptiles, etc.

As used herein, the term, “optimized” means that a nucleotide sequence has been altered to encode an amino acid sequence using codons that are preferred in the production cell or organism, either a eukaryotic cell, for example, a cell of Pichia or Saccharomyces, a cell of Trichoderma, a Chinese Hamster Ovary cell (CHO) or a human cell, or a prokaryotic cell, for example, a strain of Escherichia coli.

The optimized nucleotide sequence is engineered to retain completely or as much as possible the amino acid sequence originally encoded by the starting nucleotide sequence, which is also known as the “parental” sequence. The optimized sequences herein have been engineered to have codons that are preferred in the corresponding production cell or organism, for example a mammalian cell, however optimized expression of these sequences in other prokaryotic or eukaryotic cells is also envisioned herein. The amino acid sequences encoded by optimized nucleotide sequences are also referred to as optimized.

As used herein, a “SIRPα binding domain” refers to any single chain polypeptide domain that is necessary for binding to SIRPα under appropriate conditions. A SIRPα binding domain comprises all amino acid residues directly involved in the physical interaction with SIRPα. It may further comprise other amino acids that do not directly interact with SIRPα but are required for the proper conformation of the SIRPα binding domain to interact with SIRPα. SIRPα binding domains may be fused to heterologous domains without significant alteration of their binding properties to SIRPα. SIRPα binding domain may be selected among the binding domains of proteins known to bind to SIRPα such as the CD47 protein. The SIRPα binding domain may further consist of artificial binders to SIRPα. In particular, binders derived from single chain immunoglobulin scaffolds, such as single domain antibody, single chain antibody (scFv) or camelid antibody. In one embodiment, the term “SIRPα binding domain” does not contain SIRPα antigen-binding regions derived from variable regions, such as V_{H} and V_{L} regions of an antibody that binds to SIRPα.

Various aspects of the invention are described in further detail in the following subsections.

Preferred embodiments of the Extended Fusobodies of the invention are soluble SIRPα binding proteins, complexes thereof, and derivatives all of which comprise SIRPα-binding domain as described hereafter. For ease of reading, Extended Fusobodies, complexes thereof, and derivatives, comprising SIRPα binding domains are referred to as the SIRPα binding Proteins of the Invention.

In one preferred embodiment, the SIRPα binding domain is selected from the group consisting of:
(i) an extracellular domain of human CD47;
(ii) a polypeptide of SEQ ID NO:4 or a fragment of SEQ ID NO:4 retaining SIRPα binding properties;
(iii) a variant polypeptide of SEQ ID NO:4 having at least 60, 70, 80, 90, 95, 96, 97, 98, or 99 percent sequence identity to SEQ ID NO:4 and retaining SIRPα binding properties;
(iv) a polypeptide of SEQ ID NO:3 or a fragment of SEQ ID NO:3 retaining SIRPα binding properties;
(v) a variant polypeptide of SEQ ID NO:3 having at least 60, 70, 80, 90, 95, 96, 97, 98, or 99 percent sequence identity to SEQ ID NO:3 and retaining SIRPα binding properties;
(vi) a polypeptide of SEQ ID NO:57 or a fragment of SEQ ID NO:57 retaining SIRPα binding properties; and,
(vii) a variant polypeptide of SEQ ID NO:57 having at least 60, 70, 80, 90, 95, 96, 97, 98, or 99 percent sequence identity to SEQ ID NO:57 and retaining SIRPα binding properties.

The SIRPα binding proteins of the invention should retain the capacity to bind to SIRPα. The binding domain of CD47 has been well characterized and one extracellular domain of human CD47 is a polypeptide of SEQ ID NO:4, SEQ ID NO:57 or SEQ ID NO:3. Fragments of the polypeptide of SEQ ID NO:4, SEQ ID NO:57 or SEQ ID NO:3 can therefore be selected among those fragments comprising the SIRPα binding domain of CD47. Those fragments generally do not comprise the transmembrane and intracellular domains of CD47. In non-limiting illustrative embodiments, SIRPα-binding domains essentially consist of SEQ ID NO:3, SEQ ID NO:4, SEQ ID NO:5, or SEQ ID NO:57. Fragments include without limitation shorter polypeptides wherein between 1 and 10 amino acids have been truncated from C-terminal or N-terminal of SEQ ID NO:3, SEQ ID NO:4 or SEQ ID NO:5, for example SEQ ID NO:57. SIRPα-binding domains further include, without limitation, a variant polypeptide of SEQ ID NO:4, SEQ ID NO:57 or SEQ ID NO:3, where amino acids residues have been mutated by amino acid deletion, insertion or substitution, yet have at least 60, 70, 80, 90, 95, 96, 97, 98, or 99 percent identity to SEQ ID NO:4, SEQ ID NO:57 or SEQ ID NO:3, respectively; so long as changes to the native sequence do not substantially affect the biological activity of the SIRPα binding proteins, in particular its binding properties to SIRPα. In some embodiments, it includes mutant amino acid sequences wherein no more than 1, 2, 3, 4 or 5 amino acids have been mutated by amino acid deletion or substitution in the SIRPα-binding domain when compared with SEQ ID NO:4, SEQ ID NO:57 or SEQ ID NO:3. Examples of mutant amino acid sequences are those sequences derived from single nucleotide polymorphisms (see Table 2).

As used herein, the percent identity between two sequences is a function of the number of identical positions shared by the sequences (i.e., % identity=# of identical positions/total # of positions ×100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino acid sequences can be determined using the algorithm of E. Myers and W. Miller (Comput. Appl. Biosci. 4:11-17, 1988) which has been incorporated into the ALIGN program. In addition, the percent identity between two amino acid sequences can be determined using the Needleman and Wunsch (J. Mol. Biol. 48:443-453, 1970) algorithm which has been incorporated into the GAP program in the GCG software package. Yet another program to determine percent identity is CLUSTAL (M. Larkin et al., Bioinformatics 23:2947-2948, 2007; first described by D. Higgins and P. Sharp, Gene 73:237-244, 1988) which is available as stand-alone program or via web servers (see http://www.clustal.org/).

In a specific embodiment, the SIRPα binding domain includes changes to SEQ ID NO:4, SEQ ID NO:57 or SEQ ID NO:3 wherein said changes to SEQ ID NO:4, SEQ ID NO:57 or SEQ ID NO:3 essentially consist of conservative amino acid substitutions.

Conservative amino acid substitutions are ones in which the amino acid residue is replaced with an amino acid residue having a similar side chain. Families of amino acid residues having similar side chains have been defined in the art. These families include amino acids with basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine, tryptophan), nonpolar side chains (e.g. alanine, valine, leucine, isoleucine, proline, phenylalanine, methionine), beta-branched side chains (e.g. threonine, valine, isoleucine) and aromatic side chains (e.g. tyrosine, phenylalanine, tryptophan, histidine). Thus, one or more amino acid residues within the SIRPα binding domain of SEQ ID NO:4, SEQ ID NO:57 or SEQ ID NO:3 can be replaced with other amino acid residues from the same side chain family, and the new polypeptide variant can be tested for retained function using the binding or functional assays described herein.

In another embodiment, the SIRPα binding domains are selected among those that cross-react with non-human primate SIRPα such as cynomolgus or rhesus monkeys.

In another embodiment, the SIRPα binding domains are selected among those that do not cross-react with human proteins closely related to SIRPα, such as SIRPγ.

In some embodiments, the SIRPα binding domains are selected among those that retain the capacity for a SIRPα-binding Protein that comprises such SIRPα binding domain, to inhibit the binding of CD47-Fc fusion to SIRPα+U937 cells, at least to the same extent as a SIRPα binding Protein comprising the extracellular domain of human SIRPα of SEQ ID NO:4 or SEQ ID NO:3, as measured in a plate-based cellular adhesion assay.

In other embodiments, the SIRPα binding domains are selected among those that retain the capacity for a SIRPα-binding Protein, that comprises such SIRPα binding domain, to inhibit Staphylococcus aureus Cowan strain particles stimulated release of proinflammatory cytokines in in vitro differentiated myeloid dendritic cells, at least to the same extent as a SIRPα binding Protein comprising the extracellular domain of human SIRPα of SEQ ID NO:4 or SEQ ID NO:3 as measured in a dendritic cell cytokine release assay.

The SIRPα binding domain can be fused directly in frame with the VH or VL regions or via a polypeptidic linker (spacer). Such spacer may be a single amino acid (such as, for example, a glycine residue) or between 5-100 amino acids, for example between 5-20 amino acids. The linker should permit the SIRPα binding domain to assume the proper spatial orientation to form a binding site with SIRPα. Suitable polypeptide linkers may be selected among those that adopt a flexible conformation. Examples of such linkers are (without limitation) those linkers comprising Glycine and Serine residues, for example, (Gly₄Ser)ₙ wherein n is an integer between 1-12, for example between 1 and 4, for example 2.

SIRPα binding Proteins of the invention can be conjugated or fused together to form multivalent proteins.

The skilled person can further advantageously use the background technologies developed for engineering antibody molecules, either to increase the valencies of the molecule, or improve or adapt the properties of the engineered molecules for their specific use.

In another embodiment, SIRPα binding Proteins of the invention can be fused to another heterologous protein, which is capable of increasing half-life of the resulting fusion protein in blood.

Such heterologous protein can be, for example, an immunoglobulin, serum albumin and fragments thereof. Such heterologous protein can also be a polypeptide capable of binding to serum albumin proteins to increase half life of the resulting molecule when administered in a subject. Such approach is for example described in EP0486525.

Alternatively or in addition, the soluble proteins of the invention further comprise a domain for multimerization.

### SIRPα Binding Extended Fusobody

In one aspect, the invention relates to an Extended Fusobody comprising at least one SIRPα binding domain. The two heterodimers of the Extended Fusobody may contain different binding domains with different binding specificities, thereby resulting in a bi- or trispecific Fusobody. For example, the Fusobody may comprise one heterodimer containing SIRPα binding domain and another heterodimer containing another heterologous binding domain. Alternatively, both heterodimers of the Fusobody comprise SIRPα binding domains. In the latter, the structure or amino acid sequence of such SIRPα binding domains may be identical or different. In one preferred embodiment, both heterodimers of the Fusobody comprise identical SIRPα binding domains.

### Specific Examples of SIRPα Binding Extended Fusobodies of the Invention

Fusobodies of the invention include without limitation the Fusobodies structurally characterized as described in Table 4 in the Examples. The SIRPα binding domain used in these examples is shown in SEQ ID NO:3 or SEQ ID NO:4. Specific examples of heavy chain amino acid sequences of SIRPα binding Extended Fusobodies of the invention are polypeptide sequences selected from the group consisting of: SEQ ID NO:20, SEQ ID NO:22 and SEQ ID NO:40. Specific examples of light chain amino acid sequences of SIRPα binding Extended Fusobodies of the invention are polypeptide sequences selected from the group consisting of: SEQ ID NO:21, SEQ ID NO:23 and SEQ ID NO:41.

Other SIRPα binding Extended Fusobodies of the invention comprise SIRPα binding domains that have been mutated by amino acid deletion, insertion or substitution, yet have at least 60, 70, 80, 90, 95, 96, 97, 98, or 99 percent sequence identity in any one of the corresponding SIRPα binding domains of SEQ ID NO:3 or SEQ ID NO:4. In some embodiments, Fusobodies of the invention comprise SIRPα binding domains which include mutant amino acid sequences wherein no more than 1, 2, 3, 4 or 5 amino acids have been changed by amino acid deletion or substitution in the SIRPα binding domains when compared with the SIRPα binding domains as depicted in any one of the sequences SEQ ID NO: SEQ ID NO:3 or SEQ ID NO:4.

In one embodiment, a SIRPα binding Extended Fusobody of the invention, described as Example #4, comprises a first single heavy chain polypeptide of SEQ ID NO:18 and a second single light chain polypeptide of SEQ ID NO:19.

In one embodiment, a SIRPα binding Extended Fusobody of the invention, described as Example #5, comprises a first single heavy chain polypeptide of SEQ ID NO:20 and a second single light chain polypeptide of SEQ ID NO:21.

In one embodiment, a SIRPα binding Extended Fusobody of the invention, described as Example #6, comprises a first single heavy chain polypeptide of SEQ ID NO:22 and a second single light chain polypeptide of SEQ ID NO:23.

In one embodiment, a SIRPα binding Extended Fusobody of the invention, described as Example #7, comprises a first single heavy chain polypeptide of SEQ ID NO:40 and a second single light chain polypeptide of SEQ ID NO:41.

In one embodiment, a SIRPα binding Extended Fusobody of the invention comprises a heavy chain polypeptide and/or light chain polypeptide having at least 95 percent sequence identity to at least one of the corresponding heavy chain and or light chain polypeptides of Example #4, #5, #6, or #7 above.

In another aspect, the invention provides an isolated Extended Fusobody of the invention, described as Example #4, having: a first single heavy chain polypeptide encoded by a nucleotide sequence of SEQ ID NO:75; and a second single light chain polypeptide encoded by a nucleotide sequence of SEQ ID NO:76.

In another aspect, the invention provides an isolated Extended Fusobody of the invention, described as Example #5, having: a first single heavy chain polypeptide encoded by a nucleotide sequence of SEQ ID NO:77; and a second single light chain polypeptide encoded by a nucleotide sequence of SEQ ID NO:78.

In another aspect, the invention provides an isolated Extended Fusobody of the invention, described as Example #6, having: a first single heavy chain polypeptide encoded by a nucleotide sequence of SEQ ID NO:79; and a second single light chain polypeptide encoded by a nucleotide sequence of SEQ ID NO:80.

In another aspect, the invention provides an isolated Extended Fusobody of the invention, described as Example #7, having: (iii) a first single heavy chain polypeptide encoded by a nucleotide sequence of SEQ ID NO:97; and a second single light chain polypeptide encoded by a nucleotide sequence of SEQ ID NO:98.

Other SIRPα binding Extended Fusobodies of the invention comprise a heavy chain encoded by nucleotide sequences which have been mutated by nucleotide deletion, insertion or substitution, yet have at least 60, 70, 80, 90, 95, 96, 97, 98, or 99 percent sequence identity to SEQ ID NO:77, or SEQ ID NO:79 or SEQ ID NO:97. In some embodiments, Extended Fusobodies of the invention comprise a heavy chain encoded by a nucleotide sequence which includes mutant nucleotide sequence wherein no more than 1, 2, 3, 4 or 5 nucleotides have been changed by nucleotide deletion, insertion or substitution when compared with SEQ ID NO:77, or SEQ ID NO:79 or SEQ ID NO:97. The SIRPα binding Extended Fusobodies of the invention comprise a light chain encoded by nucleotide sequences which have been mutated by nucleotide deletion, insertion or substitution, yet have at least 60, 70, 80, 90, 95, 96, 97, 98, or 99 percent sequence identity to SEQ ID NO:78, or SEQ ID NO:80 or SEQ ID NO:98. In some embodiments, Extended Fusobodies of the invention comprise a light chain encoded by a nucleotide sequence which includes mutant nucleotide sequence wherein no more than 1, 2, 3, 4 or 5 nucleotides have been changed by nucleotide deletion, insertion or substitution when compared with SEQ ID NO:78, or SEQ ID NO:80 or SEQ ID NO:98.

In preferred embodiments, the invention provides an isolated Extended Fusobody of the invention, wherein (a) the VH region comprises one or more CDRS selected from the group consisting of: SEQ ID NO:27, SEQ ID NO:28, and SEQ ID NO:29 and/or the VL region comprises one or more CDRS selected from the group consisting of: SEQ ID NO:31, SEQ ID NO:32 and SEQ ID NO:33, or (b) the VH region comprises one or more CDRS selected from the group consisting of: SEQ ID NO:45, SEQ ID NO:46, and SEQ ID NO:47 and/or the VL region comprises one or more CDRS selected from the group consisting of: SEQ ID NO:49, SEQ ID NO:50 and SEQ ID NO:51, or (c) the VH and/or VL regions comprises one or more CDRs sharing at least 60, 70, 80, 90, 95, 96, 97, 98, or 99 percent sequence identity with the corresponding CDR sequences as described in (a) or (b) above.

In preferred embodiments an Extended Fusobody of the invention comprises (a) a VH polypeptide sequence selected from the group consisting of: SEQ ID NO:26 and SEQ ID NO:44, and/or (b) a VL polypeptide sequence selected from the group consisting of: SEQ ID NO:30 and SEQ ID NO:48, and/or (c) a VH or VL polypeptide sequence having at least 95 percent sequence identity to at least one of the corresponding VH or VL sequences as described in (a) or (b) above.

In a preferred aspect, the invention further provides an Extended Fusobody, which cross-blocks or is cross-blocked by at least one Soluble Protein or Extended Fusobody as described previously, or which competes for binding to the same epitope as a Soluble Protein or Extended Fusobody as described previously.

### Functional Fusobodies

In yet another embodiment, a SIRPα binding Extended Fusobody of the invention has heavy and light chain amino acid sequences; heavy and light chain nucleotide sequences or SIRPα binding domains fused to heavy and light chain constant regions, that are homologous to the corresponding amino acid and nucleotide sequences of the specific SIRPα binding Fusobodies described in the above paragraph, in particular, Examples #4, #5 #6 and #7 as described in Table 4, and wherein said Extended Fusobodies retain substantially the same functional properties of at least one of the specific SIRPα binding Fusobodies described in the above paragraph, in particular, Examples #4-7 as described in Table 4.

For example, the invention provides an isolated Extended Fusobody comprising a heavy chain amino acid sequence and a light chain amino acid sequence, wherein: the heavy chain has an amino acid sequence that is at least 80%, at least 90%, at least 95% or at least 99% identical to an amino acid sequence selected from the group consisting of SEQ ID NO: 20, SEQ ID NO:22, and SEQ ID NO:40; the light chain has an amino acid sequence that is at least 80%, at least 90%, at least 95% or at least 99% identical to an amino acid sequence selected from the group consisting of SEQ ID NO:21, SEQ ID NO:23, and SEQ ID NO:41; the Extended Fusobody specifically binds to SIRPα, and either TNFalpha or cyclosporin A, and the Extended Fusobody inhibits Staphylococcus aureus Cowan strain particles stimulated release of proinflammatory cytokines in in vitro generated monocyte derived dendritic cells.

As used herein, an Extended Fusobody that “specifically binds to SIRPα” is intended to refer to a Fusobody that binds to human SIRPα polypeptide of SEQ ID NO:1 with a k_{off} (kd1) of 0.05 [1/s] or less, within at least one of the binding affinity assays described in the Examples, for example by surface plasmon resonance in a BiaCORE assay. An Extended Fusobody that “cross-reacts with a polypeptide other than SIRPα” is intended to refer to a Fusobody that binds that other polypeptide with a k_{off} (kd1) of 0.05 [1/s] or less. An Extended Fusobody that “does not cross-react with a particular polypeptide” is intended to refer to a Fusobody that binds to that polypeptide, with a with a k_{off} (kd1) at least ten fold higher, preferably at least hundred fold higher than the k_{off} (kd1) measuring binding affinity of said Extended Fusobody to human SIRPα under similar conditions. In certain embodiments, such Fusobodies that do not cross-react with the other polypeptide exhibit essentially undetectable binding against these proteins in standard binding assays.

In various embodiments, the Fusobody may exhibit one or more or all of the functional properties discussed above.

In other embodiments, the SIRPα-binding domains may be 50%, 60%, 70%, 80%, 90%, 95%, 96%, 97%, 98% or 99% identical to at least one of the specific sequences of SIRPα binding domains set forth in the above paragraph related to “SIRPα binding domains”, including without limitation a polypeptide of SEQ ID NO:3, SEQ ID NO:4, SEQ ID NO:5, SEQ ID NO:57 or a fragment thereof retaining SIRPα binding properties. In other embodiments, the SIRPα-binding domains may be identical to at least one of the specific sequences of SIRPα binding domains set forth in the above paragraph related to “SIRPα binding domains”, including without limitation a polypeptide of SEQ ID NO:3, SEQ ID NO:4, SEQ ID NO:5, SEQ ID NO:57 or a fragment thereof retaining SIRPα binding properties, except for an amino acid substitution in no more than 1, 2, 3, 4 or 5 amino acid positions of said specific sequence.

An Extended Fusobody having SIRPα-binding domains with high (i.e., at least 80%, 90%, 95%, 99% or greater) identity to specifically described SIRPα-binding domains, can be obtained by mutagenesis (e.g. site-directed or PCR-mediated mutagenesis) of nucleic acid molecules encoding said specific SIRPα-binding domains respectively, followed by testing of the encoded altered Extended Fusobody for retained function (i.e. the functions set forth above) using the functional assays described herein.

In other embodiments, the heavy chain and light chain amino acid sequences may be 50% 60%, 70%, 80%, 90%, 95%, 96%, 97%, 98% or 99% identical to the heavy and light chains of the specific Fusobody Examples #4-7 set forth above, while retaining at least one of the functional properties of SIRPα binding Extended Fusobody described above. A SIRPα binding Extended Fusobody having a heavy chain and light chain having high (i.e. at least 80%, 90%, 95% or greater) identity to the corresponding heavy chains of any of SEQ ID NO:20, or SEQ ID NO:22 or SEQ ID NO:40 and light chains of any of SEQ ID NO:21, or SEQ ID NO:23 or SEQ ID NO:41, respectively, can be obtained by mutagenesis (e.g. site-directed or PCR-mediated mutagenesis) of nucleic acid molecules encoding heavy chains SEQ ID NO: 77, SEQ ID NO:79, and SEQ ID NO:97; and light chains SEQ ID NO:78, SEQ ID NO:80 and SEQ ID NO:98; respectively, followed by testing of the encoded altered SIRPα binding Fusobody for retained function (i.e., the functions set forth above) using the functional assays described herein.

In one embodiment, a SIRPα binding Extended Fusobody of the invention is a variant of Example #4, having a heavy chain at least 80%, 90%, 95% or 99% identical to SEQ ID NO:18 and a light chain at least 80%, 90%, 95% or 99% identical to SEQ ID NO:19, the Extended Fusobody specifically binds to SIRPα, and the Extended Fusobody inhibits release of proinflammatory cytokines in in vitro generated monocyte-derived dendritic cells elicited by various bacterial derivatives such as Staphylococcus aureus Cowan strain particles or others.

In one embodiment, a SIRPα binding Extended Fusobody of the invention is a variant of Example #5, having a heavy chain at least 80%, 90%, 95% or 99% identical to SEQ ID NO:20 and a light chain at least 80%, 90%, 95% or 99% identical to SEQ ID NO:21, the Extended Fusobody specifically binds to SIRPα, and the Extended Fusobody exhibits at least one of the following functional properties: (i) it inhibits release of proinflammatory cytokines in in vitro generated monocyte-derived dendritic cells elicited by various bacterial derivatives such as Staphylococcus aureus Cowan strain particles or others, and (ii) it has binding specificity for TNF alpha.

In one embodiment, a SIRPα binding Extended Fusobody of the invention is a variant of Example #6, having a heavy chain at least 80%, 90%, 95% or 99% identical to SEQ ID NO:22 and a light chain at least 80%, 90%, 95% or 99% identical to SEQ ID NO:23, the Extended Fusobody specifically binds to SIRPα, and the Extended Fusobody exhibits at least one of the following functional properties: (i) it inhibits release of proinflammatory cytokines in in vitro generated monocyte-derived dendritic cells elicited by various bacterial derivatives such as Staphylococcus aureus Cowan strain particles or others, and (ii) it has binding specificity for TNF alpha.

In one embodiment, a SIRPα binding Extended Fusobody of the invention is a variant of Example #7, having a heavy chain at least 80%, 90%, 95% or 99% identical to SEQ ID NO:40 and a light chain at least 80%, 90%, 95% or 99% identical to SEQ ID NO:41, the Extended Fusobody specifically binds to SIRPα, and the Extended Fusobody exhibits at least one of the following functional properties: (i) it inhibits release of proinflammatory cytokines in in vitro generated monocyte-derived dendritic cells elicited by various bacterial derivatives such as Staphylococcus aureus Cowan strain particles or others, and (ii) it has binding specificity for cyclosporin A.

### Fc Domain of Extended Fusobody

An Fc domain comprises at least the C_{H}2 and C_{H}3 domain. As used herein, the term Fc domain further includes, without limitation, Fc variants into which an amino acid substitution, deletion or insertion at one, two, three, four of five amino acid positions has been introduced compared to natural Fc fragment of antibodies, for example, human Fc fragments.

The use of Fc domain for making soluble constructs with increased in vivo half life in human is well known in the art and for example described in Capon et al. (U.S. Pat. No. 5,428,130). In one embodiment, it is proposed to use a similar Fc moiety within a Fusobody construct. However, it is appreciated that the invention does not relate to known proteins of the Art sometimes referred as “Fc fusion proteins” or “immunoadhesin”. Indeed, the term “Fc fusion proteins” or “immunoadhesins” generally refer in the Art to a heterologous binding region directly fused to C_{H}2 and C_{H}3 domain, but which does not comprise at least either of C_{L} or C_{H}1 region. The resulting protein comprises two heterologous binding regions. The Fusobody may comprise an Fc moiety fused to the N-terminal of the C_{H}1 region, thereby reconstituting a full length constant heavy chain which can interact with a light chain, usually via C_{H}1 and C_{L} disulfide bonding.

In one embodiment, the hinge region of C_{H}1 of the Extended Fusobody or SIRPα binding Proteins is modified such that the number of cysteine residues in the hinge region is altered, e.g. increased or decreased. This approach is described further in U.S. Pat. No. 5,677,425 (Bodmer et al.). The number of cysteine residues in the hinge region of C_{H}1 is altered to, for example, facilitate assembly of the light and heavy chains or to increase or decrease the stability of the fusion polypeptide.

In another embodiment, the Fc region of the Extended Fusobody or SIRPα binding Proteins is modified to increase its biological half-life. Various approaches are possible. For example, one or more of the following positions can be mutated: 252, 254, 256, as described in U.S. Pat. No. 6,277,375, for example: M252Y, S254T, T256E.

In yet other embodiments, the Fc region of the Extended Fusobody or SIRPα binding Proteins is altered by replacing at least one amino acid residue with a different amino acid residue to alter the effector functions of the Fc portion. For example, one or more amino acids can be replaced with a different amino acid residue such that the Fc portion has an altered affinity for an effector ligand. The effector ligand to which affinity is altered can be, for example, an Fc receptor or the C1 component of complement. This approach is described in further detail in U.S. Pat. Nos. 5,624,821 and 5,648,260, both by Winter et al.

In another embodiment, one or more amino acids selected from amino acid residues can be replaced with a different amino acid residue such that the resulting Fc portion has altered C1q binding and/or reduced or abolished complement dependent cytotoxicity (CDC). This approach is described in further detail in U.S. Pat. No. 6,194,551 (Idusogie et al.)

In another embodiment, one or more amino acid residues are altered to thereby alter the ability of the Fc region to fix complement. This approach is described further in PCT Publication WO 94/29351 by Bodmer et al.

In yet another embodiment, the Fc region of the Extended Fusobody or SIRPα binding Proteins is modified to increase the ability of the fusion polypeptide to mediate antibody dependent cellular cytotoxicity (ADCC) and/or to increase or decrease the affinity of the Fc region for an Fcγ receptor by modifying one or more amino acids. This approach is described further in PCT Publication WO 00/42072. Moreover, the binding sites on human IgG1 for FcγRI, FcγRII, FcγRIII and FcRn have been mapped and variants with improved binding have been described (see Shields, R. L. et al., 2001 J. Biol. Chem. 276:6591-6604).

In one embodiment, the Fc domain of the Extended Fusobody or SIRPα binding Proteins is of human origin and may be from any of the immunoglobulin classes, such as IgG or IgA and from any subtype such as human IgG1, IgG2, IgG3 and IgG4 or chimera of IgG1, IgG2, IgG3 and IgG4. In other embodiments the Fc domain is from a non-human animal, for example, but not limited to, a mouse, rat, rabbit, camelid, shark, non-human primate or hamster.

In certain embodiments, the Fc domain of IgG1 isotype is used in the Extended Fusobody or SIRPα binding Proteins. In some specific embodiments, a mutant variant of IgG1Fc fragment is used, e.g. a silent IgG1Fc which reduces or eliminates the ability of the fusion polypeptide to mediate antibody dependent cellular cytotoxicity (ADCC) and/or to bind to an Fcγ receptor. An example of an IgG1 isotype silent mutant, is a so-called LALA mutant, wherein leucine residues are replaced by alanine residues at amino acid positions 234 and 235, as described by Hezareh et al. (J. Virol 2001 December; 75(24):12161-8). Another example of an IgG1 isotype silent mutant comprises the D265A mutation, and/or the P329A mutation. In certain embodiments, the Fc domain is a mutant preventing glycosylation at residue at position 297 of Fc domain, for example, an amino acid substitution of asparagine residue at position 297 of the Fc domain. Example of such amino acid substitution is the replacement of N297 by a glycine or an alanine.

In another embodiment, the Fc domain is derived from IgG2, IgG3 or IgG4.

In one embodiment, the Fc domain of the Extended Fusobody or SIRPα binding Proteins comprises a dimerization domain, preferably via cysteine capable of making covalent disulfide bridge between two fusion polypeptides comprising such Fc domain.

### Glycosylation Modifications

In still another embodiment, the glycosylation pattern of the soluble proteins of the invention, including in particular the SIRPα-binding Proteins or Extended Fusobodies, can be altered compared to typical mammalian glycosylation pattern such as those obtained in CHO or human cell lines. For example, an aglycoslated protein can be made by using prokaryotic cell lines as host cells or mammalian cells that has been engineered to lack glycosylation. Carbohydrate modifications can also be accomplished by; for example, altering one or more sites of glycosylation within the SIRPα binding protein or Extended Fusobody.

Additionally or alternatively, a glycosylated protein can be made that has an altered type of glycosylation. Such carbohydrate modifications can be accomplished by, for example, expressing the soluble proteins of the invention in a host cell with altered glycosylation machinery, i.e the glycosylation pattern of the soluble protein is altered compared to the glycosylation pattern observed in corresponding wild type cells. Cells with altered glycosylation machinery have been described in the art and can be used as host cells in which to express recombinant soluble proteins to thereby produce such soluble proteins with altered glycosylation. For example, EP 1,176,195 (Hang et al.) describes a cell line with a functionally disrupted FUT8 gene, which encodes a fucosyl transferase, such that glycoproteins expressed in such a cell line exhibit hypofucosylation. WO 03/035835 describes a variant CHO cell line, Lecl3 cells, with reduced ability to attach fucose to Asn(297)-linked carbohydrates, also resulting in hypofucosylation of glycoproteins expressed in that host cell (see also Shields, R. L. et al., 2002 J. Biol. Chem. 277:26733-26740). Alternatively, the soluble proteins can be produced in yeast, e.g. Pichia pastoris, or filamentous fungi, e.g. Trichoderma reesei, engineered for mammalian-like glycosylation pattern (see for example EP1297172B1). Advantages of those glycoengineered host cells are, inter alia, the provision of polypeptide compositions with homogeneous glycosylation pattern and/or higher yield.

### Pegylated Soluble Proteins and Other Conjugates

Another embodiment of the soluble proteins or the invention relates to pegylation. The soluble proteins of the invention, for example, SIRPα-binding Proteins or Extended Fusobodies can be pegylated. Pegylation is a well-known technology to increase the biological (e.g. serum) half-life of the resulting biologics as compared to the same biologics without pegylation. To pegylate a polypeptide, the polypeptide is typically reacted with polyethylene glycol (PEG), such as a reactive ester or aldehyde derivative of PEG, under conditions in which one or more PEG groups become attached to the polypeptides. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. Methods for pegylating proteins are known in the art and can be applied to the soluble proteins of the invention. See for example, EP 0 154 316 by Nishimura et al. and EP 0 401 384 by Ishikawa et al.

Alternative conjugates or polymeric carrier can be used, in particular to improve the pharmacokinetic properties of the resulting conjugates. The polymeric carrier may comprise at least one natural or synthetic branched, linear or dendritic polymer. The polymeric carrier is preferably soluble in water and body fluids and is preferably a pharmaceutically acceptable polymer. Water soluble polymer moieties include, but are not limited to, e.g. polyalkylene glycol and derivatives thereof, including PEG, PEG homopolymers, mPEG, polypropyleneglycol homopolymers, copolymers of ethylene glycol with propylene glycol, wherein said homopolymers and copolymers are unsubstituted or substituted at one end e.g. with an acylgroup; polyglycerines or polysialic acid; carbohydrates, polysaccharides, cellulose and cellulose derivatives, including methylcellulose and carboxymethylcellulose; starches (e.g. hydroxyalkyl starch (HAS), especially hydroxyethyl starch (HES) and dextrines, and derivatives thereof; dextran and dextran derivatives, including dextransulfat, crosslinked dextrin, and carboxymethyl dextrin; chitosan (a linear polysaccharide), heparin and fragments of heparin; polyvinyl alcohol and polyvinyl ethyl ethers; polyvinylpyrrollidon; alpha, beta-poly[(2-hydroxyethyl)-DL-aspartamide; and polyoxy-ethylated polyols.

### Use of the SIRPα Binding Proteins as a Medicament

The Extended Fusobodies and in particular the SIRPα binding soluble proteins of the invention may be used as a medicament, in particular to decrease or suppress (in a statistically or biologically significant manner) the inflammatory and/or autoimmune response, in particular, a response mediated by SIRPα+ cells in a subject. When conjugated to cytotoxic agents or with cell-killing effector functions provided by Fc moiety, the SIRPα binding can also be advantageously used in treating, decrease or suppress cancer disorders or tumors, such as, in particular myeloid lymphoproliferative diseases such as acute myeloid lymphoproliferative (AML) disorders or bladder cancer.

### Nucleic Acid Molecules Encoding the Soluble Proteins of the Invention

Another aspect of the invention pertains to nucleic acid molecules that encode the soluble proteins of the invention, including without limitation, the embodiments related to Extended Fusobodies, for example as described in Table 4 of the Examples. The invention provides an isolated nucleic acid encoding at least one single chain polypeptide of one heterodimer of the soluble protein. Non-limiting examples of nucleotide sequences encoding the SIRPα binding Extended Fusobodies comprise SEQ ID NO:77 and SEQ ID NO:78; or SEQ ID NO:79 and SEQ ID NO:80; or SEQ ID NO:97 and SEQ ID NO:98, each pair encoding respectively the heavy and light chains of a SIRPα binding Extended Fusobody.

The nucleic acids may be present in whole cells, in a cell lysate, or may be nucleic acids in a partially purified or substantially pure form. A nucleic acid is “isolated” or “rendered substantially pure” when purified away from other cellular components or other contaminants, e.g. other cellular nucleic acids or proteins, by standard techniques, including alkaline/SDS treatment, CsCl banding, column chromatography, agarose gel electrophoresis and others well known in the art. See, F. Ausubel, et al., ed. 1987 Current Protocols in Molecular Biology, Greene Publishing and Wiley Interscience, New York. A nucleic acid of the invention can be, for example, DNA or RNA and may or may not contain intronic sequences. In an embodiment, the nucleic acid is a cDNA molecule. The nucleic acid may be present in a vector such as a phage display vector, or in a recombinant plasmid vector. The invention thus provides an isolated nucleic acid or a cloning or expression vector comprising at least one nucleic acid selected from the group consisting of: SEQ ID NO:77, SEQ ID NO:78, SEQ ID NO:79, SEQ ID NO:80, SEQ ID NO:97, and SEQ ID NO:98.

DNA fragments encoding the soluble SIRPα binding proteins or Extended Fusobodies, as described above and in the Examples, can be further manipulated by standard recombinant DNA techniques, for example to include any signal sequence for appropriate secretion in expression system, any purification tag and cleavable tag for further purification steps. In these manipulations, a DNA fragment is operatively linked to another DNA molecule, or to a fragment encoding another protein, such as a purification/secretion tag or a flexible linker. The term “operatively linked”, as used in this context, is intended to mean that the two DNA fragments are joined in a functional manner, for example, such that the amino acid sequences encoded by the two DNA fragments remain in-frame, or such that the protein is expressed under control of a desired promoter.

### Generation of Transfectomas Producing the SIRPα-Binding Proteins and Extended Fusobodies

The soluble proteins of the invention, for example SIRPα-binding proteins or Extended Fusobodies can be produced in a host cell transfectoma using, for example, a combination of recombinant DNA techniques and gene transfection methods as is well known in the art. For expressing and producing recombinant Extended Fusobodies in host cell transfectoma, the skilled person can advantageously use its own general knowledge related to the expression and recombinant production of antibody molecules or antibody-like molecules. The invention provides a recombinant host cell suitable for the production of a soluble protein or protein complex of the invention, comprising the nucleic acids encoding said first and second single chain polypeptides of said heterodimers of said protein, and optionally, secretion signals.

In one aspect the recombinant host cell comprises the nucleic acids of SEQ ID NO:77 and SEQ ID NO:78; or SEQ ID NO:79 and SEQ ID NO:80; or SEQ ID NO:97 and SEQ ID NO:98 stably integrated in the genome. In a preferred aspect the host cell is a mammalian cell line. The invention provides a process for the production of a soluble protein, such as a SIRPα-binding protein or Extended Fusobody, or a protein complex of the invention, as described previously, comprising culturing the host cell under appropriate conditions for the production of the soluble protein or protein complex, and isolating said protein.

For example, to express the soluble proteins of the invention or intermediates thereof, DNAs encoding the corresponding polypeptides, can be obtained by standard molecular biology techniques (e.g. PCR amplification or cDNA cloning using a hybridoma that expresses the polypeptides of interest) and the DNAs can be inserted into expression vectors such that the corresponding gene is operatively linked to transcriptional and translational control sequences. The expression vector and expression control sequences are chosen to be compatible with the expression host cell used. The gene encoding the soluble proteins of the invention, e.g. the heavy and light chains of the SIRPα binding Extended Fusobodies or intermediates are inserted into the expression vector by standard methods (e.g. ligation of complementary restriction sites on the gene fragment and vector, or blunt end ligation if no restriction sites are present). Additionally or alternatively, the recombinant expression vector can encode a signal peptide that facilitates secretion of the polypeptide chain(s) from a host cell. The gene can be cloned into the vector such that the signal peptide is linked in frame to the amino terminus of the polypeptide chain. In specific embodiments with CD47 derived sequences as SIRPα binding region, the signal peptide can be a CD47 signal peptide or a heterologous signal peptide (i.e. a signal peptide not naturally associated with CD47 sequence).

In addition to the polypeptide encoding sequence, the recombinant expression vectors of the invention carry regulatory sequences that control the expression of the gene in a host cell. The term “regulatory sequence” is intended to include promoters, enhancers and other expression control elements (e.g. polyadenylation signals) that control the transcription or translation of the polypeptide chain genes. Such regulatory sequences are described, for example, in Goeddel (Gene Expression Technology, Methods in Enzymology 185, Academic Press, San Diego, Calif. 1990). It will be appreciated by those skilled in the art that the design of the expression vector, including the selection of regulatory sequences, may depend on such factors as the choice of the host cell to be transformed, the level of expression of protein desired, etc. Regulatory sequences for mammalian host cell expression include viral elements that direct high levels of protein expression in mammalian cells, such as promoters and/or enhancers derived from cytomegalovirus (CMV), Simian Virus 40 (SV40), adenovirus (e.g. the adenovirus major late promoter (AdMLP)), and polyoma. Alternatively, nonviral regulatory sequences may be used, such as the ubiquitin promoter or P-globin promoter.

Still further, regulatory elements composed of sequences from different sources, such as the SRa promoter system, which contains sequences from the SV40 early promoter and the long terminal repeat of human T cell leukemia virus type 1 (Takebe, Y. et al., 1988 Mol. Cell. Biol. 8:466-472).

In addition to this, the recombinant expression vectors of the invention may carry additional sequences, such as sequences that regulate replication of the vector in host cells (e.g. origins of replication) and selectable marker genes. The selectable marker gene facilitates selection of host cells into which the vector has been introduced (see, e.g. U.S. Pat. Nos. 4,399,216; 4,634,665; and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr-host cells with methotrexate selection/amplification) and the neo gene (for G418 selection).

For expression of the protein, the expression vector(s) encoding the soluble proteins or intermediates such as heavy and light chain sequences of the SIRPα binding Extended Fusobody is transfected into a host cell by standard techniques. The various forms of the term “transfection” are intended to encompass a wide variety of techniques commonly used for the introduction of exogenous DNA into a prokaryotic or eukaryotic host cell, e.g. electroporation, calcium-phosphate precipitation, DEAE-dextran transfection and the like. It is theoretically possible to express the soluble proteins of the invention in either prokaryotic or eukaryotic host cells. Expression of glycoprotein in eukaryotic cells, in particular mammalian host cells, is discussed because such eukaryotic cells, and in particular mammalian cells, are more likely than prokaryotic cells to assemble and secrete a properly folded and biologically active glycoprotein such as the SIRPα binding Extended Fusobodies.

The Extended Fusobodies can be advantageously produced using well known expression systems developed for antibodies molecules. One of the advantages of the Extended Fusobodies of the invention over prior art molecules which comprise dual variable domains is that the antigen/target specificities can be achieved using a combination of natural or near-natural mammalian binding domain sequences together with VH and VL sequences provided by an antibody. Because the soluble proteins comprise only one set of VH and VL sequences per heterodimer, the positioning of these regions next to the associated regions of the mammalian binding molecules is less critical than that required when positioning two (or more) sets of VH and VL sequences. Thus, in terms of utilization and optimisation of any linker sequences, and further with regard to expression of the heterodimers in a host cell, the soluble proteins of the invention provide increased simplicity and ease of production, and require simpler manipulation using molecular biology. Put another way, there is less requirement to optimise the spacing of the sequences comprised within the soluble proteins of the invention and yet still retain the required functionality. This is to be contrasted with those molecules where dual specificity is achieved using two sets of VH and VL domains, where their respective conformations and positioning with respect to each other can be more critical, and which therefore requires more spatial optimisation.

Mammalian host cells for expressing the soluble proteins and intermediates such as heavy and light chains of the SIRPα binding Fusobodies of the invention include Chinese Hamster Ovary cells (CHO cells), including dhfr-CHO cells, (described by Urlaub and Chasin, 1980, Proc. Natl. Acad. Sci. USA 77:4216-4220) used with a DH FR selectable marker, e.g. as described in R. J. Kaufman and P. A. Sharp, 1982 Mol. Biol. 159:601-621), NSO myeloma cells, COS cells and SP2 cells or human cell lines (including PER-C6 cell lines, Crucell or HEK293 cells, Yves Durocher et al., 2002, Nucleic acids research vol 30, No 2 e9). When recombinant expression vectors encoding polypeptides are introduced into mammalian host cells, the soluble proteins and intermediates such as heavy and light chains of the SIRPα-binding Extended Fusobodies of the invention are produced by culturing the host cells for a period of time sufficient to allow for expression of the recombinant polypeptides in the host cells or secretion of the recombinant polypeptides into the culture medium in which the host cells are grown. The polypeptides can then be recovered from the culture medium using standard protein purification methods.

### Multivalent SIRPα Binding Proteins

In another aspect, the present invention provides multivalent proteins, for example in the form of a complex, comprising at least two identical or different soluble SIRPα binding proteins of the invention. In one embodiment, the multivalent protein comprises at least two, three or four soluble SIRPα binding proteins of the invention. The soluble SIRPα binding proteins can be linked together via protein fusion or covalent or non-covalent linkages. The multivalent proteins of the present invention can be prepared by conjugating the constituent binding specificities, using methods known in the art. For example, each binding specificity of the multivalent protein can be generated separately and then conjugated to one another.

A variety of coupling or cross-linking agents can be used for covalent conjugation. Examples of cross-linking agents include protein A, carbodiimide, N-succinimidyl-5-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohaxane-1-carboxylate (sulfo-SMCC) (see e.g. Karpovsky et al., 1984 J. Exp. Med. 160:1686; Liu, M A et al., 1985 Proc. Natl. Acad. Sci. USA 82:8648). Other methods include those described in Paulus, 1985 Behring Ins. Mitt. No. 78, 118-132;

Brennan et al., 1985 Science 229:81-83), and Glennie et al., 1987 J. Immunol. 139: 2367-2375). Covalent linkage can be obtained by disulfide bridge between two cysteines, for example disulfide bridge from cysteine of an Fc domain.

### Conjugated SIRPα Binding Proteins

In another aspect, the present invention features an Extended Fusobody, in particular a SIRPα binding Extended Fusobody, conjugated to a therapeutic moiety, such as a cytotoxin, a drug (e.g. an immunosuppressant) or a radiotoxin. Such conjugates are referred to herein as “Conjugated Extended Fusobodies” or “Conjugated SIRPα binding Extended Fusobodies”. A cytotoxin or cytotoxic agent includes any agent that is detrimental to (e.g. kills) cells. Such agents have been used to prepare conjugates of antibodies or immunoconjugates. Such technologies can be applied advantageously with Conjugated Extended Fusobodies, in particular Conjugated SIRPα binding Extended Fusobodies. Examples of cytotoxin or cytotoxic agent include taxon, cytochalasin B, gramicidin D, ethidium bromide, emetine, mitomycin, etoposide, tenoposide, vincristine, vinblastine, t. colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g. methotrexate, 6-mercaptopurine, 6-thioguanine, cytarabine, 5-fluorouracil decarbazine), ablating agents (e.g. mechlorethamine, thioepa chloraxnbucil, meiphalan, carmustine (BSNU) and lomustine (CCNU), cyclothosphamide, busulfan, dibromomannitol, streptozotocin, mitomycin C, and cis-dichlorodiamine platinum (II) (DDP) cisplatin, anthracyclines (e.g. daunorubicin (formerly daunomycin) and doxorubicin), antibiotics (e.g. dactinomycin (formerly actinomycin), bleomycin, mithramycin, and anthramycin (AMC)), and anti-mitotic agents (e.g. vincristine and vinblastine).

Other examples of therapeutic cytotoxins that can be conjugated to the Extended Fusobodies of the invention include duocarmycins, calicheamicins, maytansines and auristatins, and derivatives thereof.

Cytoxins can be conjugated to SIRPα binding Proteins or Extended Fusobodies of the invention using linker technology available in the art. Examples of linker types that have been used to conjugate a cytotoxin to SIRPα binding Proteins or Extended Fusobodies of the invention include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g. cathepsins B, C, D).

For further discussion of types of cytotoxins, linkers and methods for conjugating therapeutic agents to antibodies, see also Saito, G. et al., 2003 Adv. Drug Deliv. Rev. 55:199-215; Trail, P. A. et al., 2003 Cancer Immunol. Immunother. 52:328-337; Payne, G., 2003 Cancer Cell 3:207-212; Allen, T. M., 2002 Nat. Rev. Cancer 2:750-763; Pastan, I. and Kreitman, R. J., 2002 Curr. Opin. Investig. Drugs 3:1089-1091; Senter, P. D. and Springer, C. J., 2001 Adv. Drug Deliv. Rev. 53:247-264.

SIRPα binding Proteins or Extended Fusobodies of the present invention also can be conjugated to a radioactive isotope to generate cytotoxic radiopharmaceuticals. Examples of radioactive isotopes that can be conjugated to the SIRPα binding Proteins or Extended Fusobodies of the present invention for use diagnostically or therapeutically include, but are not limited to, iodineI31, indium111, yttrium90, and lutetium177. Method for preparing radioimmunconjugates are established in the art. Examples of radioimmunoconjugates are commercially available, including Zevalin™ (DEC Pharmaceuticals) and Bexxar™ (Corixa Pharmaceuticals), and similar methods can be used to prepare radiopharmaceuticals using SIRPα binding Proteins or Extended Fusobodies of the present invention of the invention. Furthermore, techniques for conjugating toxin or radioisotopes to antibodies are well known, see, e.g. Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

### Pharmaceutical Compositions

In another aspect, the present invention provides a composition, e.g. a pharmaceutical composition, containing one or a combination of the soluble SIRPα binding proteins or Extended Fusobodies of the present invention, formulated together with one or more pharmaceutically acceptable vehicles or carriers.

Pharmaceutical formulations comprising a soluble SIRPα binding protein or Extended Fusobody of the invention may be prepared for storage by mixing the proteins having the desired degree of purity with optional physiologically acceptable carriers, excipients or stabilizers (Remington: The Science and Practice of Pharmacy 20th edition (2000)), in the form of aqueous solutions, lyophilized or other dried formulations. The invention further relates to a lyophilized composition comprising at least the soluble protein of the invention, e.g. the SIRPα binding Extended Fusobodies of the invention and one or more appropriate pharmaceutically acceptable carriers. The invention also relates to syringes pre-filled with a liquid formulation comprising at least the soluble protein of the invention, e.g. the SIRPα binding Extended Fusobodies, and one or more appropriate pharmaceutically acceptable carriers or vehicles.

The pharmaceutical composition may additionally comprise at least one other active ingredient. Thus, pharmaceutical compositions of the invention also can be administered in combination therapy, i.e., combined with other agents. For example, the combination therapy can include a soluble SIRPα binding protein or Extended Fusobody of the present invention combined with at least one other active ingredient, such as an anti-inflammatory or another chemotherapeutic agent. Examples of therapeutic agents that can be used in combination therapy are described in greater detail below in the section on uses of the soluble SIRPα binding proteins of the invention.

As used herein, “pharmaceutically acceptable carrier” or “pharmaceutically acceptable vehicle” includes any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g. by injection or infusion). Depending on the route of administration, the active principle may be coated in a material to protect it from the action of acids and other natural conditions that may inactivate the active principle.

The pharmaceutical composition of the invention may include one or more pharmaceutically acceptable salts. A “pharmaceutically acceptable salt” refers to a salt that retains the desired biological activity of the parent compound and does not impart any undesired toxicological effects (see e.g. Berge, S. M., et al., 1977 J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and di-carboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as, aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, one can include isotonic agents, for example, sugars, polyalcohols such as mannitol, sorbitol, or sodium chloride in the composition. Prolonged absorption of the injectable compositions can be brought about by including in the composition an agent that delays absorption for example, monostearate salts and gelatin.

Sterile injectable solutions can be prepared by incorporating the soluble proteins, e.g. the SIRPα binding Extended Fusobodies in the required amount in an appropriate solvent with one or a combination of ingredients enumerated above, as required, followed by sterilization microfiltration. Generally, dispersions are prepared by incorporating the active principle into a sterile vehicle that contains a basic dispersion medium and the required other ingredients from those enumerated above. In the case of sterile powders for the preparation of sterile injectable solutions, the methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

Dosage regimens are adjusted to provide the optimum desired response (e.g. a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals.

For administration of the soluble SIRPα binding proteins or Extended Fusobodies of the invention, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-30 mg/kg. An exemplary treatment regime entails administration once per week, once every two weeks, once every three weeks, once every four weeks, once a month, once every 3 months or once every three to 6 months. Dosage regimens for a soluble SIRPα binding proteins or Extended Fusobodies of the invention include 1 mg/kg body weight or 3 mg/kg body weight by intravenous administration, with the protein being given using one of the following dosing schedules: every four weeks for six dosages, then every three months; every three weeks; 3 mg/kg body weight once followed by 1 mg/kg body weight every three weeks.

The soluble SIRPα binding proteins or Extended Fusobodies are usually administered on multiple occasions. Intervals between single dosages can be, for example, weekly, monthly, every three months or yearly. Intervals can also be irregular as indicated by measuring blood levels of soluble polypeptide/protein in the patient. In some methods, dosage is adjusted to achieve a plasma polypeptide concentration of about 0.1-1000 μg/ml and in some methods about 5-300 μg/ml.

Alternatively, the soluble SIRPα binding proteins or Extended Fusobodies can be administered as a sustained release formulation, in which case less frequent administration is required. Dosage and frequency vary depending on the half-life of the soluble proteins in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated or until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of soluble SIRPα binding proteins or Extended Fusobodies can result in a decrease in severity of disease symptoms, an increase in frequency and duration of disease symptom-free periods, or a prevention of impairment or disability due to the disease affliction.

A composition of the present invention can be administered by one or more routes of administration using one or more of a variety of methods known in the art. As will be appreciated by the skilled artisan, the route and/or mode of administration will vary depending upon the desired results. Routes of administration for Soluble Proteins of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, intraocular, transtracheal, subcutaneous, subcuticular, intraarticular, subcapsular, subarachnoid, intraspinal, epidural and intrastemal injection and infusion.

Alternatively, soluble SIRPα binding proteins or Extended Fusobodies can be administered by a nonparenteral route, such as a topical, epidermal or mucosal route of administration, for example, intranasally, orally, vaginally, rectally, sublingually or topically.

The active principles can be prepared with carriers that will protect the proteins against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are published or generally known to those skilled in the art. See, e.g. Sustained and Controlled Release Drug Delivery Systems, J. R. Robinson, ed., Marcel Dekker, Inc., New York, 1978.

Therapeutic compositions can be administered with medical devices known in the art. For example, in one embodiment, a therapeutic composition of the invention can be administered with a needleless hypodermic injection device, such as the devices shown in U.S. Pat. Nos. 5,399,163; 5,383,851; 5,312,335; 5,064,413; 4,941,880; 4,790,824 or 4,596,556. Examples of well known implants and modules useful in the present invention include: U.S. Pat. No. 4,487,603, which shows an implantable micro-infusion pump for dispensing medication at a controlled rate; U.S. Pat. No. 4,486,194, which shows a therapeutic device for administering medicants through the skin; U.S. Pat. No. 4,447,233, which shows a medication infusion pump for delivering medication at a precise infusion rate; U.S. Pat. No. 4,447,224, which shows a variable flow implantable infusion apparatus for continuous drug delivery; U.S. Pat. No. 4,439,196, which shows an osmotic drug delivery system having multi-chamber compartments; and U.S. Pat. No. 4,475,196, which shows an osmotic drug delivery system. Many other such implants, delivery systems, and modules are known to those skilled in the art.

In certain embodiments, the soluble SIRPα binding proteins or Extended Fusobodies can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g. U.S. Pat. Nos. 4,522,811; 5,374,548; and 5,399,331. The liposomes may comprise one or more moieties which are selectively transported into specific cells or organs, thus enhance targeted drug delivery (see, e.g. V. V. Ranade, 1989 J. Cline Pharmacol. 29:685).

### Uses and Methods of the Invention

The soluble SIRPα binding proteins or Extended Fusobodies have in vitro and in vivo diagnostic and therapeutic utilities. For example, these molecules can be administered to cells in culture, e.g. in vitro or in vivo, or in a subject, e.g. in vivo, to treat, prevent or diagnose a variety of disorders. In one embodiment, the soluble SIRPα binding proteins or Extended Fusobodies can be used in in vitro expansion of stem cells or other cell types like pancreatic beta cells in the presence of other cell types which otherwise would interfere with expansion. In addition, in particular the soluble SIRPα binding proteins or Extended Fusobodies are used to in vitro qualify and quantify the expression of functional SIRPα at the cell surface of cells from a biological sample of an organism such as human. This application may be useful as commercially available SIRPα antibodies cross-react with various isoforms of SIRPβ making difficult to unambigously quantify SIRPα protein expression on the cell surface. Quantification of soluble SIRPα binding Proteins or Extended Fusobodies can therefore be used for diagnostic purpose for example to assess the correlation of the quantity of SIRPα protein expression with immune or cancer disorders and therefore allow selection of patients (patient stratification) for treatment with, for example, conjugated SIRPα binding proteins or antibody-based therapies targeted against SIRPα

The methods are particularly suitable for treating, preventing or diagnosing autoimmune and inflammatory disorders mediated by SIRPα+ cells e.g. allergic asthma or ulcerative colitis. These include acute and chronic inflammatory conditions, allergies and allergic conditions, autoimmune diseases, ischemic disorders, severe infections, and cell or tissue or organ transplant rejection including transplants of non-human tissue (xenotransplants). The methods are particularly suitable for treating, preventing or diagnosing autoimmune and inflammatory or malignant disorders mediated by cells expressing aberrant or mutated variants of the activating SIRPβ receptor which are reactive to CD47 and dysfunction via binding to CD47 or other SIRPα ligands.

Examples of autoimmune diseases include, without limitation, arthritis (for example rheumatoid arthritis, arthritis chronica progrediente and arthritis deformans) and rheumatic diseases, including inflammatory conditions and rheumatic diseases involving bone loss, inflammatory pain, spondyloarhropathies including ankolsing spondylitis, Reiter syndrome, reactive arthritis, psoriatic arthritis, and enterophathis arthritis, hypersensitivity (including both airways hypersensitivity and dermal hypersensitivity) and allergies. Autoimmune diseases include autoimmune haematological disorders (including e.g. hemolytic anaemia, aplastic anaemia, pure red cell anaemia and idiopathic thrombocytopenia), systemic lupus erythematosus, inflammatory muscle disorders, polychondritis, sclerodoma, Wegener granulomatosis, dermatomyositis, chronic active hepatitis, myasthenia gravis, psoriasis, Steven-Johnson syndrome, idiopathic sprue, endocrine ophthalmopathy, Graves disease, sarcoidosis, multiple sclerosis, primary biliary cirrhosis, juvenile diabetes (diabetes mellitus type I), uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including gout, langerhans cell histiocytosis, idiopathic nephrotic syndrome or minimal change nephropathy), tumors, multiple sclerosis, inflammatory disease of skin and cornea, myositis, loosening of bone implants, metabolic disorders, such as atherosclerosis, diabetes, and dislipidemia.

The soluble SIRPα binding proteins or Extended Fusobodies are also useful for the treatment, prevention, or amelioration of asthma, bronchitis, pneumoconiosis, pulmonary emphysema, and other obstructive or inflammatory diseases of the airways.

The soluble SIRPα binding proteins or Extended Fusobodies are also useful for the treatment, prevention, or amelioration of immune system-mediated or inflammatory myopathies including coronar myopathies.

The soluble SIRPα binding proteins or Extended Fusobodies are also useful for the treatment, prevention, or amelioration of disease involving the endothelial or smooth muscle system which express SIRPα.

The soluble SIRPα binding proteins or Extended Fusobodies are also useful for the treatment of IgE-mediated disorders. IgE mediated disorders include atopic disorders, which are characterized by an inherited propensity to respond immunologically to many common naturally occurring inhaled and ingested antigens and the continual production of IgE antibodies. Specific atopic disorders include allergic asthma, allergic rhinitis, atopic dermatitis and allergic gastroenteropathy.

However, disorders associated with elevated IgE levels are not limited to those with an inherited (atopic) etiology. Other disorders associated with elevated IgE levels, that appear to be IgE-mediated and are treatable with the formulations of this present invention include hypersensitivity (e.g. anaphylactic hypersensitivity), eczema, urticaria, allergic bronchopulmonary aspergillosis, parasitic diseases, hyper-IgE syndrome, ataxia-telangiectasia, Wiskott-Aldrich syndrome, thymic alymphoplasia, IgE myeloma and graft-versus-host reaction.

The soluble SIRPα binding proteins or Extended Fusobodies are useful as first line treatment of acute diseases involving the major nervous system in which inflammatory pathways are mediated by SIRPα+ cells such as activated microglia cells. A particular application for instance can be the silencing of activated microglia cells after spinal cord injury to accelerate healing and prevent the formation of lymphoid structures and antibodies autoreactive to parts of the nervous system.

The soluble SIRPα binding proteins or Extended Fusobodies may be administered as the sole active ingredient or in conjunction with, e.g. as an adjuvant to or in combination to, other drugs e.g. immunosuppressive or immunomodulating agents or other anti-inflammatory agents, e.g. for the treatment or prevention of diseases mentioned above. For example, the soluble SIRPα binding proteins or Extended Fusobodies may be used in combination with DMARD, e.g. Gold salts, sulphasalazine, antimalarias, methotrexate, D-penicillamine, azathioprine, mycophenolic acid, cyclosporine A, tacrolimus, sirolimus, minocycline, leflunomide, glococorticoids; a calcineurin inhibitor, e.g. cyclosporin A or FK 506; a modulator of lymphocyte recirculation, e.g. FTY720 and FTY720 analogs; a mTOR inhibitor, e.g. rapamycin, 40-O-(2-hydroxyethyl)-rapamycin, CCI779, ABT578, AP23573 or TAFA-93; an ascomycin having immuno-suppressive properties, e.g. ABT-281, ASM981, etc.; corticosteroids; cyclo-phos-phamide; azathioprene; methotrexate; leflunomide; mizoribine; mycophenolic acid; myco-pheno-late mofetil; 15-deoxyspergualine or an immunosuppressive homologue, analogue or derivative thereof; immunosuppressive monoclonal antibodies, e.g. monoclonal antibodies to leukocyte receptors, e.g. MHC, CD2, CD3, CD4, CD7, CD8, CD25, CD28, CD40. CD45, CD58, CD80, CD86 or their ligands; other immunomodulatory compounds, e.g. LEA29Y; adhesion molecule inhibitors, e.g. LFA-1 antagonists, ICAM-1 or -3 antagonists, VCAM-4 antagonists or VLA-4 antagonists; or a chemotherapeutic agent, e.g. paclitaxel, gemcitabine, cisplatinum, doxorubicin or 5-fluorouracil; anti TNF agents, e.g. monoclonal antibodies to TNF, e.g. infliximab, adalimumab, CDP870, or receptor constructs to TNF-RI or TNF-RII, e.g. Etanercept, PEG-TNF-RI; blockers of proinflammatory cytokines, IL-1 blockers, e.g. Anakinra or IL-1 trap, AAL160, ACZ 885, IL-6 blockers; chemokines blockers, e.g inhibitors or activators of proteases, e.g. metalloproteases, anti-IL-15 antibodies, anti-IL-6 antibodies, anti-CD20 antibodies, anti-CD22 antibodies, anti-IL17 antibodies, anti-IL12 antibodies, anti-IL12R antibodies, anti-IL23 antibodies, anti-IL23R antibodies, anti-IL21 antibodies, NSAIDs, such as aspirin, ibuprophen, paracetamol, naproxen, selective Cox2 inhibitors, combined Cox1 and 2 inhibitors like diclofenac, or an anti-infectious agent (list not limited to the agent mentioned).

The soluble SIRPα binding proteins or Extended Fusobodies are also useful as co-therapeutic agents for use in conjunction with anti-inflammatory or bronchodilatory drug substances, particularly in the treatment of obstructive or inflammatory airways diseases such as those mentioned hereinbefore, for example as potentiators of therapeutic activity of such drugs or as a means of reducing required dosaging or potential side effects of such drugs. An agent of the invention may be mixed with the anti-inflammatory or bronchodilatory drug in a fixed pharmaceutical composition or it may be administered separately, before, simultaneously with or after the anti-inflammatory or bronchodilatory drug. Such anti-inflammatory drugs include steroids, in particular glucocorticosteroids such as budesonide, beclamethasone, fluticasone or mometasone, and dopamine receptor agonists such as cabergoline, bromocriptine or ropinirole. Such bronchodilatory drugs include anticholinergic or antimuscarinic agents, in particular ipratropium bromide, oxitropium bromide and tiotropium bromide.

Combinations of agents of the invention and steroids may be used, for example, in the treatment of COPD or, particularly, asthma. Combinations of agents of the invention and anticholinergic or antimuscarinic agents or dopamine receptor agonists may be used, for example, in the treatment of asthma or, particularly, COPD.

In accordance with the foregoing, the present invention also provides a method for the treatment of an obstructive or inflammatory airways disease which comprises administering to a subject, particularly a human subject, in need thereof a soluble SIRPα binding protein or Extended Fusobody, as hereinbefore described. In another aspect, the invention provides a soluble SIRPα binding Protein or Extended Fusobody, as hereinbefore described for use in the preparation of a medicament for the treatment of an obstructive or inflammatory airways disease.

The soluble SIRPα binding proteins or Extended Fusobodies are also particularly useful for the treatment, prevention, or amelioration of chronic gastrointestinal inflammation, such as inflammatory bowel diseases, including Crohn's disease and ulcerative colitis.

“Chronic gastrointestinal inflammation” refers to inflammation of the mucosal of the gastrointestinal tract that is characterized by a relatively longer period of onset, is long-lasting (e.g. from several days, weeks, months, or years and up to the life of the subject), and is associated with infiltration or influx of mononuclear cells and can be further associated with periods of spontaneous remission and spontaneous occurrence. Thus, subjects with chronic gastrointestinal inflammation may be expected to require a long period of supervision, observation, or care. “Chronic gastrointestinal inflammatory conditions” (also referred to as “chronic gastrointestinal inflammatory diseases”) having such chronic inflammation include, but are not necessarily limited to, inflammatory bowel disease (IBD), colitis induced by environmental insults (e.g. gastrointestinal inflammation (e.g. colitis) caused by or associated with (e.g. as a side effect) a therapeutic regimen, such as administration of chemotherapy, radiation therapy, and the like), colitis in conditions such as chronic granulomatous disease (Schappi et al. Arch Dis Child. 2001 February; 1984(2):147-151), celiac disease, celiac sprue (a heritable disease in which the intestinal lining is inflamed in response to the ingestion of a protein known as gluten), food allergies, gastritis, infectious gastritis or enterocolitis (e.g. Helicobacter pylori-infected chronic active gastritis) and other forms of gastrointestinal inflammation caused by an infectious agent, and other like conditions.

As used herein, “inflammatory bowel disease” or “IBD” refers to any of a variety of diseases characterized by inflammation of all or part of the intestines. Examples of inflammatory bowel disease include, but are not limited to, Crohn's disease and ulcerative colitis. Reference to IBD throughout the specification is often referred to in the specification as exemplary of gastrointestinal inflammatory conditions, and is not meant to be limiting.

In accordance with the foregoing, the present invention also provides a method for the treatment of chronic gastrointestinal inflammation or inflammatory bowel diseases, such as ulcerative colitis, which comprises administering to a subject, particularly a human subject, in need thereof, a soluble SIRPα binding Protein or Extended Fusobody, as hereinbefore described. In another aspect, the invention provides a soluble SIRPα binding protein or Extended Fusobody, as hereinbefore described for use in the preparation of a medicament for the treatment of chronic gastrointestinal inflammation or inflammatory bowel diseases.

The present invention is also useful in the treatment, prevention or amelioration of leukemias or other cancer disorders. For example, the soluble SIRPα binding proteins of the invention could induce cell depletion or apoptosis in leukemias. A soluble SIRPα binding protein or Extended Fusobody can be used in treating, preventing or ameliorating cancer disorders selected from acute myeloid leukemia, acute lymphoblastic leukemia, chronic myeloid leukemia, chronic lymphocytic leukemia, myeloproliferative disorders, myelodysplastic syndromes, multiple myeloma, non-Hodgkin lymphoma, hodgkin disease, bladder cancer, malignant forms of langerhans cell histiocytosis.

Modulating SIRPα-CD47 interaction can be used to increase hematopoietic stem cell engraftment (see e.g. WO2009/046541 related to the use of CD47-Fc fusion proteins). The present invention, and for example, soluble SIRPα binding proteins or Extended Fusobodies are therefore useful for increasing human hematopoietic stem cell engraftment. Hematopoietic stem cell engraftment can be used to treat or reduce symptoms of a patient that is suffering from impaired hematopoiesis or from an inherited immunodeficient disease, an autoimmune disorder or hematopoietic disorder, or having received any myelo-ablative treatment. For example, such hematopoietic disorder is selected from acute myeloid leukemia, acute lymphoblastic leukemia, chronic myeloid leukemia, chronic lymphocytic leukemia, myeloproliferative disorders, myelodysplastic syndromes, multiple myeloma, non-Hodgkin lymphoma, hodgkin disease, aplastic anemia, pure red cell aplasia, paroxysmal nocturnal hemoglobinuria, fanconi anemi, thalassemia major, Sickle cell anemia, severe combined immunodeficiency, Wiskott-Aldrich syndrome, hemophagocytic lymphohistiocytosis and inborn errors of metabolism. Therefore, in one embodiment, the invention relates to Soluble SIRPα binding Proteins or Fusobodies for use in treating hematopoietic disorder is selected from acute myeloid leukemia, acute lymphoblastic leukemia, chronic myeloid leukemia, chronic lymphocytic leukemia, myeloproliferative disorders, myelodysplastic syndromes, multiple myeloma, non-Hodgkin lymphoma, hodgkin disease, aplastic anemia, pure red cell aplasia, paroxysmal nocturnal hemoglobinuria, fanconi anemi, thalassemia major, Sickle cell anemia, severe combined immunodeficiency, Wiskott-Aldrich syndrome, hemophagocytic lymphohistiocytosis and inborn errors of metabolism in particular, after treatment with an expanded cell population containing hematopoietic stem cell, in order to improve hematopoietic stem cell engraftment.

Also encompassed within the scope of the present invention is a method as defined above comprising co-administration, e.g. concomitantly or in sequence, of a therapeutically effective amount of a soluble SIRPα binding protein or Extended Fusobody, and at least one second drug substance, said second drug substance being an immuno-suppressive/immunomodulatory, anti-inflammatory chemotherapeutic or anti-infectious drug, e.g. as indicated above.

Also encompassed within the scope of the present invention is a therapeutic combination, e.g. a kit, comprising of a therapeutically effective amount of a) a soluble SIRPα binding protein or Extended Fusobody and b) at least one second substance selected from an immuno-suppressive/immunomodulatory, anti-inflammatory chemotherapeutic or anti-infectious drug, e.g. as indicated above. The kit may comprise instructions for its administration.

Where the soluble SIRPα binding proteins or Extended Fusobodies are administered in conjunction with other immuno-suppressive/immunomodulatory, anti-inflammatory chemotherapeutic or anti-infectious therapy, dosages of the co-administered combination compound will of course vary depending on the type of co-drug employed, on the condition being treated and so forth.

### BRIEF DESCRIPTION OF THE FIGURES

FIG. 1: Schematic representation of an example of a SIRPalpha binding Extended Fusobody, compared with a non-extended Fusobody and a reference CD47-Fc molecule.

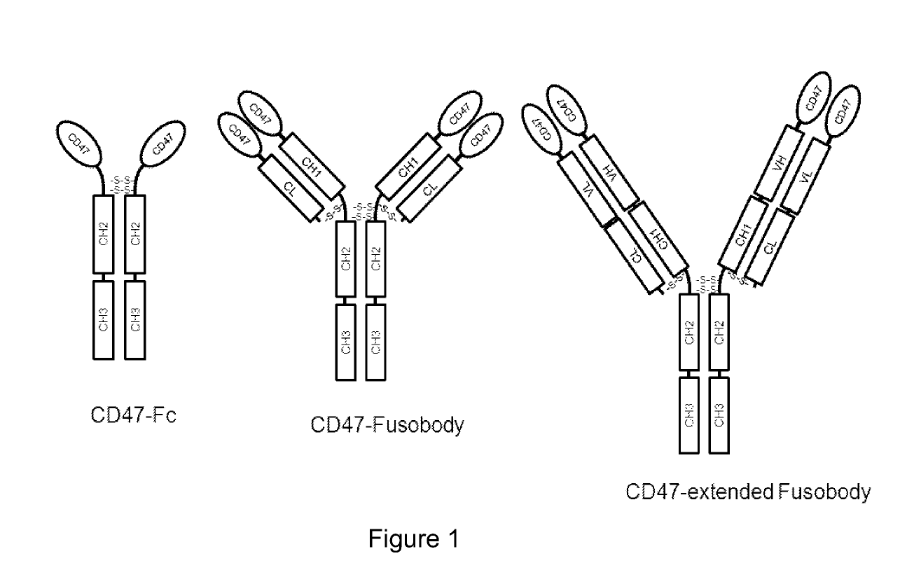

FIG. 2A: Binding of a reference CD47-Fc molecule (Example #9) to immobilized human SIRPalpha.

FIG. 2B: Binding of an Extended Fusobody having CD47 and TNFalpha specificity (Example #5) to immobilized human SIRPalpha.

FIG. 3: Binding of Extended Fusobodies having specificity for CD47 and TNFalpha (Example #5 and #6) to immobilized recombinant human TNFalpha, compared to a non-Extended Fusobody having CD47 specificity (Example #2) and an anti-TNFalpha monoclonal antibody (Example #8).

The invention having been fully described, it is further illustrated by the following examples and claims, which are illustrative and are not meant to be further limiting.

### EXAMPLES

### 1. Examples of Extended Fusobodies of the Invention

The following table 4 provides examples of Extended Fusobodies of the invention (examples #4, #5, #6, and #7) that may be produced by recombinant methods using DNA encoding the disclosed Extended Fusobody heavy and light chain amino acid sequences. The table further includes Fusobodies having a non-extended format (examples #2 and #3), and reference CD47-Fc molecules (examples #1 and #9), and a commercially available conventional anti-TNF antibody (example #8).

TABLE 4

| | | | | | | SIRPα | SEQ ID of full |
|---|---|---|---|---|---|---|---|
| | | | CH1 region or | VH or VL | | binding | length |
| Example | Description | Fc Part | CL region | region | Linker | region | polypeptide |
| #1 | CD47-Fc | SEQ ID | Not applicable | Not | Not | SEQ ID | SEQ ID NO: 7 |
| | reference | NO: 6 | | applicable | applicable | NO: 4 | |
| | molecule | | | | | | |
| | (CD47 ECD fused | | | | | | |
| | to IgG1 LALA Fc | | | | | | |
| #2 | Heavy chain of | SEQ ID | SEQ ID NO: 10 | Not | SEQ ID | SEQ ID | SEQ ID NO: 14 |
| | non-extended | NO: 11 | | applicable | NO: 9 | NO: 5 | |
| | Fusobody | | | | | | |
| | (CD47 C15G- | | | | | | |
| | [G4S]₂ linker- | | | | | | |
| | CH1-IgG1 LALA | | | | | | |
| | Fc) | | | | | | |
| #2 | Light chain of | Not | SEQ ID NO: 13 | Not | SEQ ID | SEQ ID | SEQ ID NO: 15 |
| | non-extended | applicable | | applicable | NO: 9 | NO: 5 | |
| | Fusobody | | | | | | |
| | (CD47 C15G- | | | | | | |
| | [G4S]₂ linker-CL | | | | | | |
| | (human, kappa) | | | | | | |
| #3 | Heavy chain of | SEQ ID | SEQ ID NO: 10 | Not | SEQ ID | SEQ ID | SEQ ID NO: 16 |
| | non-extended | NO: 11 | | applicable | NO: 9 | NO: 4 | |
| | Fusobody with | | | | | | |
| | WT CD47 | | | | | | |
| | domains | | | | | | |
| | (CD47-[G4S]₂ | | | | | | |
| | linker-CH1- | | | | | | |
| | IgG1 LALA Fc) | | | | | | |
| #3 | Light chain of | Not | SEQ ID NO: 13 | Not | SEQ ID | SEQ ID | SEQ ID NO: 17 |
| | non-extended | applicable | | applicable | NO: 9 | NO: 4 | |
| | Fusobody with | | | | | | |
| | WT CD47 | | | | | | |
| | domains | | | | | | |
| | (CD47-[G4S]₂ | | | | | | |
| | linker-CL | | | | | | |
| | (human, kappa) | | | | | | |
| #4 | Heavy chain of | SEQ ID | SEQ ID NO: 10 | SEQ ID | SEQ ID | SEQ ID | SEQ ID NO: 18 |
| | Extended | NO: 11 | Linker between | NO: 10 | NO: 8 | NO: 57 | |
| | Fusobody | | CH1-CH1 | | | | |
| | 2x CH1/CL | | domains | | | | |
| | domain; | | corresponds to | | | | |
| | (CD47 truncated- | | SEQ ID NO: 120 | | | | |
| | [G4S]- | | | | | | |
| | CH1-Linker-CH1- | | | | | | |
| | IgG1 LALA Fc) | | | | | | |
| #4 | Light chain of | Not | SEQ ID NO: 13 | SEQ ID | SEQ ID | SEQ ID | SEQ ID NO: 19 |
| | Extended | applicable | Linker between | NO: 13 | NO: 8 | NO: 57 | |
| | Fusobody | | CL-CL domains | | | | |
| | 2x CH1/CL | | corresponds to | | | | |
| | domain; | | SEQ ID NO: 9 | | | | |
| | (CD47 truncated- | | | | | | |
| | [G4S]- | | | | | | |
| | CL-CL human, | | | | | | |
| | kappa) | | | | | | |
| #5 | Heavy chain of | SEQ ID | SEQ ID NO: 10 | SEQ ID | Not | SEQ ID | SEQ ID NO: 20 |
| | Extended | NO: 122 | | NO: 26 | applicable | NO: 4 | |
| | Fusobody | | | | | | |
| | huCD47(wt)-anti- | | | | | | |
| | TNFalpha- | | | | | | |
| | Fusobody | | | | | | |
| | (hlgG1wt-hkappa) | | | | | | |
| #5 | Light chain of | Not | SEQ ID NO: 13 | SEQ ID | Not | SEQ ID | SEQ ID NO: 21 |
| | Extended | applicable | | NO: 30 | applicable | NO: 4 | |
| | Fusobody | | | | | | |
| | huCD47(wt)-anti- | | | | | | |
| | TNFalpha- | | | | | | |
| | Fusobody | | | | | | |
| | (hlgG1wt-hkappa) | | | | | | |
| #6 | Heavy chain of | SEQ ID | SEQ ID NO: 10 | SEQ ID | SEQ ID | SEQ ID | SEQ ID NO: 22 |
| | Extended | NO: 122 | | NO: 26 | NO: 9 | NO: 4 | |
| | Fusobody | | | | | | |
| | 2GS linker | | | | | | |
| | huCD47(wt)- | | | | | | |
| | 2xG4S-anti- | | | | | | |
| | TNFalpha- | | | | | | |
| | Fusobody | | | | | | |
| | (hlgG1wt-hkappa) | | | | | | |
| #6 | Light chain of | Not | SEQ ID NO: 13 | SEQ ID | SEQ ID | SEQ ID | SEQ ID NO: 23 |
| | Extended | applicable | | NO: 30 | NO: 9 | NO: 3 | |
| | Fusobody | | | | | | |
| | 2GS linker | | | | | | |
| | huCD47(wt)- | | | | | | |
| | 2xG4S-anti- | | | | | | |
| | TNFalpha- | | | | | | |
| | Fusobody | | | | | | |
| | (hlgG1wt-hkappa) | | | | | | |
| #7 | Heavy chain of | SEQ ID | SEQ ID NO: 10 | SEQ ID | SEQ ID | SEQ ID | SEQ ID NO: 40 |
| | Extended | NO: 11 | | NO: 44 | NO: 9 | NO: 3 | |
| | Fusobody | | | | | | |
| | anti CSA | | | | | | |
| | backbone | | | | | | |
| | huCD47(wt)- | | | | | | |
| | 2xG4S-CSA- | | | | | | |
| | Fusobody | | | | | | |
| | (hlgG1LALA- | | | | | | |
| | hkappa) | | | | | | |
| #7 | Light chain of | Not | SEQ ID NO: 13 | SEQ ID | SEQ ID | SEQ ID | SEQ ID NO: 41 |
| | Extended | applicable | | NO: 48 | NO: 9 | NO: 3 | |
| | Fusobody | | | | | | |
| | anti CSA | | | | | | |
| | backbone | | | | | | |
| | huCD47(wt)- | | | | | | |
| | 2xG4S-CSA- | | | | | | |
| | Fusobody | | | | | | |
| | (hlgG1LALA- | | | | | | |
| | hkappa) | | | | | | |
| #8 | Heavy chain of | SEQ ID | SEQ ID NO: 54 | SEQ ID | Not | Not | SEQ ID NO: 38 |
| anti- | control anti- | NO: 56 | | NO: 26 | applicable | applicable | |
| TNFalpha | TNFalpha IgG1 | | | | | | |
| IgG | WT | | | | | | |
| #8 | Light chain of | Not | SEQ ID NO: 55 | SEQ ID | Not | Not | SEQ ID NO: 39 |
| anti- | control anti- | applicable | | NO: 30 | applicable | applicable | |
| TNFalpha | TNFalpha IgG1 | | | | | | |
| IgG | WT | | | | | | |
| #9 | CD47-Fc | SEQ ID | Not applicable | Not | Not | SEQ ID | SEQ ID |
| | reference | NO: 116 | | applicable | applicable | NO: 4 | NO: 117 |
| | molecule | | | | | | |
| | (CD47 ECD fused | | | | | | |
| | to IgG1 N297A Fc | | | | | | |

### 2. Affinity Determination

### 2.1. Binding Assay to SIRPalpha (BiaCORE Assay)

Avidity of Extended Fusobodies with SIRPalpha binding moieties to divalent recombinant SIRPalpha can be characterized by surface plasmon resonance. For this human SIRPalpha-Fc (1 μg/mL, R&D systems, UK) can be immobilized via Protein A on a BiaCORE chip alike CM5 (carboxymethylated dextran matrix) after surface activation/deactivation by standard procedures like EDC/NHS or ethanolamine respectively. Assessment can be done by contact time of injected Extended Fusobodies with SIRPalpha binding moieties for 120 s, dissociation times for 240 s and flow rates for 50 μl/min. After each injection of analyte, the chip can be regenerated with Gentle elution buffer (ThermoScientific).

### 2.2 Binding Assay to Immobilized Antigen

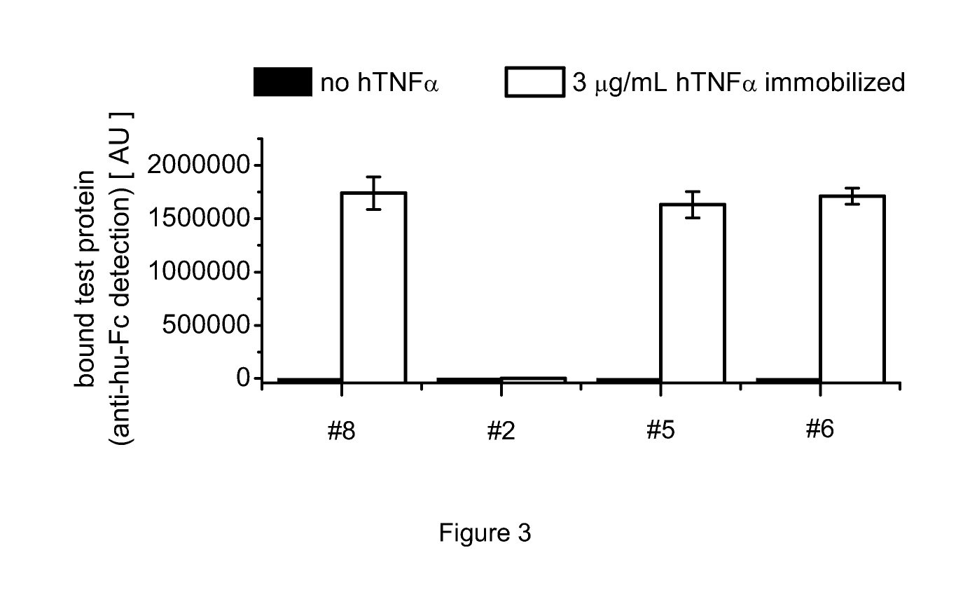

The ability of Extended Fusobodies to bind to the primary antigen of the underlying antibody-scaffold (or alternatively to the ligand of the fused-on receptor domains) can be tested by DELFIA-based methods. For the CD47-TNFalpha Extended Fusobodies (Examples #5 and #6), shown in FIG. 3, this was done by immobilizing human recombinant TNFα (Novartis inhouse or R&D systems, UK) at 1-3 μg/mL in phosphate buffered saline pH 7.6 (PBS, Life-technologies, CH) onto appropriate microtiter plates (Maxisorb, Nunc Brand, CH). After blocking with PBS containing 1% w/v bovine serum albumin (BSA), 0.05% Tween20 (Sigma Aldrich Inc, CH) test proteins are added in PBS/0.5% BSA at concentrations 0.01-1 μg/mL at room temperature on a shaker. Unbound proteins are removed by 3 wash cycles in PBS/BSA 0.5%/Tween20 0.05% followed by the addition of biotinylated goat anti-human IgG (Southern Biotech) 1-3 μg/ml. After 3 wash cycles bound biotinylated anti-human Ig is detected using Streptavidin-Europium and DELFIA detection reagents following manufacturer's instruction (Perkin Elmer). Europium-derived time resolved fluorescence can be quantified using a dedicated reader (Victor², Perkin Elmer).

### 2.3 Whole Blood Human Cell Binding Assay

Human Blood from healthy volunteers is collected into Na-Heparin coated vacutainers (BectonDickinison, BD) applying ethical guidelines. Blood is aliquoted into 96-well deep well polypropylene plates (Costar) and incubated with various concentrations of SIRPalpha binding proteins, including the Fusobodies of the present invention and reference CD47 Fc molecules, all in the presence of final 0.1% w/v sodium azide, on ice. The fluorochrome Alexa Fluor 647 (AX647) can be conjugated to the SIRPalpha binding Proteins using a labelling kit (Invitrogen). AX647-conjugated SIRPalpha binding Proteins (as described in Example 1 and table 4) can be added to the whole blood samples at a concentration of 1-10 nM for 30 min on ice. During the last 15 minutes concentration-optimized antibodies against phenotypic cell surface markers are added: CD14-PE (clone MEM18, Immunotools, Germany), CD3 Percp-Cy5.5 (clone SK7, BD), CD16 FITC (clone 3G8, BD). Whole blood is lysed by addition of 10× volume of FACSLYSING solution (BD) and incubation for 10 min at RT. Samples are washed twice with phosphate-buffered solution containing 0.5% bovine serum albumin (SIGMA-ALDRICH). Samples are acquired on a Facs Canto II (BD) within 24 hrs after lysing. Cell subsets are gated according to the monocyte light scatter profile and by CD14+ and CD3− expression. Of these cell subsets fluorescence histograms can be drawn and statistically evaluated taking the median fluoroescence intensity as readout.

### 3. Dendritic Cell Cytokine Release Assay for Measuring Inhibition of Staphylococcus aureus Cowan 1 Strain Particles Stimulated Release of Proinflammatory Cytokines

Peripheral blood monocytes (CD14+) are differentiated with GMSCF/IL4 to monocyte-derived dendritic cells (DCs) as previously described (Latour et al., J of Immunol, 2001: 167:2547). DCs are stimulated with Staphylococcus aureus Cowan 1 particles at 1/40.000 (Pansorbin) in the presence of various concentrations of human SIRPα binding Fusobodies (1 to 10000 μM) in X-VIVO15 serum-free medium. TNFalpha release is assessed by HTRF (Cisbio) after 24 h cultivation.

### 4. Results

Binding properties of the SIRPα binding Extended Fusobodies and reference molecules as described in Table 4 are presented in Tables 5A and 5B.

TABLE 5A

| | | | | | | | Improvment |
|---|---|---|---|---|---|---|---|
| | | | | | | | factor over |
| | | | Binding mode; | | | | Example #1 |
| Example | | | Valency of CD47 | IC50 | | | divalent |
| # | Format | Remark | region | nM | STDEV | N | CD47-Fc |
| 1 | CD47-Fc | Divalent CD47 Fc | Monospecific; | 99.35 | 56.31 | 13 | 1 |
| | | Reference molecule | divalent | | | | |
| 2 | Non- | huCD47C15G- | Monospecific; | 1.19 | 0.61 | 6 | 84 |
| | Extended | human IgG1 LALA- | tetravalent | | | | |
| | Fusobody | hkappa | | | | | |
| 3 | Non- | huCD47 wild type- | Monospecific; | 5.06 | 3.00 | 21 | 20 |
| | Extended | 2GS linker-human | tetravalent | | | | |
| | Fusobody | IgG1LALA-hkappa | | | | | |
| 4 | Extended | huCD47-1GS | Monospecific; | 0.87 | 0.19 | 3 | 115 |
| | Fusobody- | truncated-CH1- | tetravalent | | | | |
| | two | CH1-CH2-CH3 from | | | | | |
| | CH1/CL | human IgG1 LALA | | | | | |
| | domains | | | | | | |
| 5 | Extended | huCD47 wild type- | Bispecific; | 0.47 | 0.16 | 4 | 213 |
| | Fusobody | G4S-anti TNF | tetravalent | | | | |
| | | alpha-human | | | | | |
| | | IgG1wt-hkappa | | | | | |
| | | Extended Fusobody | | | | | |
| | | having 1GS linker | | | | | |
| 6 | Extended | huCD47 wild type- | Bispecific; | 2.50 | 1.04 | 3 | 40 |
| | Fusobody | G4SG4S-anti TNF | tetravalent | | | | |
| | | alpha-human | | | | | |
| | | IgG1wt-hkappa | | | | | |
| | | Extended Fusobody | | | | | |
| | | having 2GS linker | | | | | |
| 7 | Extended | huCD47 wild type- | Bispecific; | 5.66 | 3.35 | 3 | 18 |
| | Fusobody | G4SG4S-anti CSA- | tetravalent | | | | |
| | | human IgG1 LALA- | | | | | |
| | | hkappa | | | | | |
| | | Extended Fusobody | | | | | |
| | | with CD47 and | | | | | |
| | | cyclosporin A | | | | | |
| | | specificity | | | | | |
| 8 | Monoclon | anti-TNFalpha IgG1 | monospecific; | >1350 | | 2 | |
| | al antibody | wild type | bivalent | | | | |

### 4.1 Affinity Determination

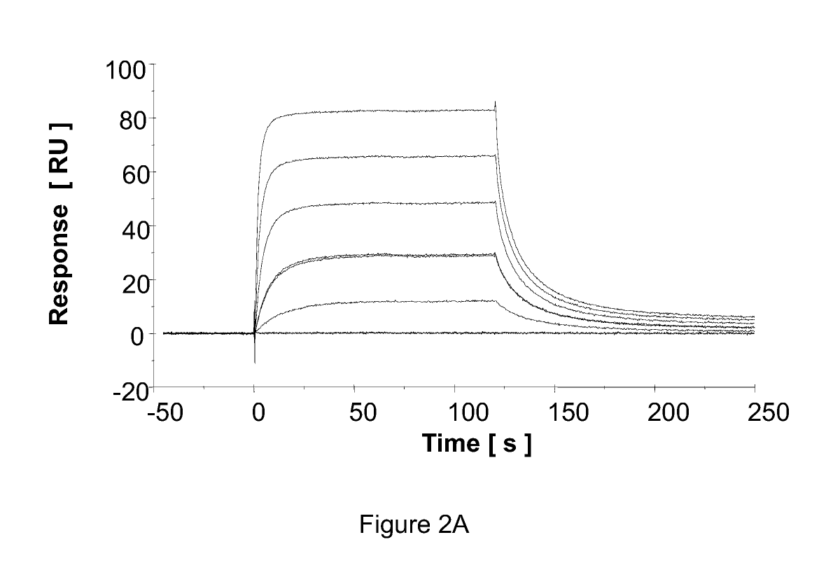

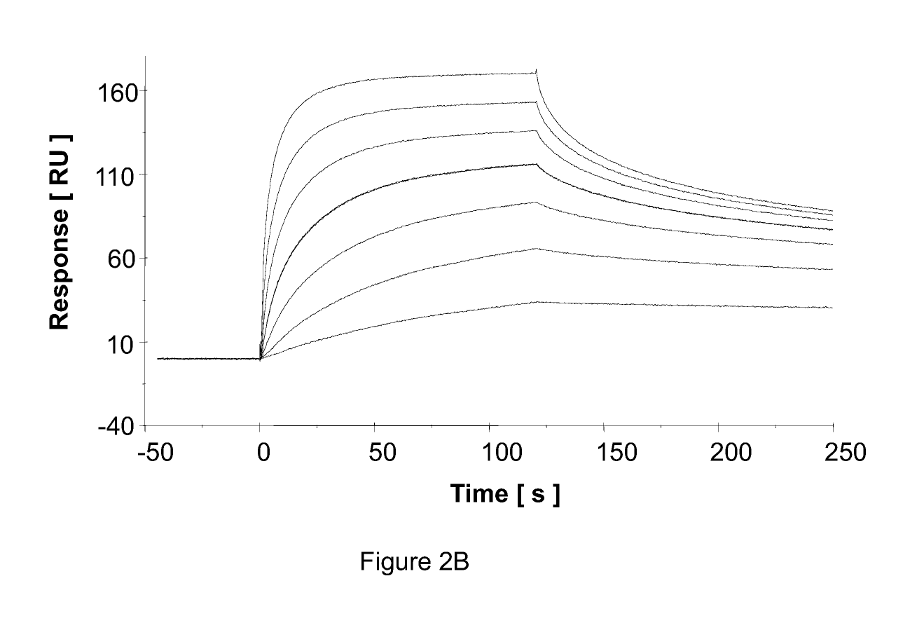

BiaCORE binding data (Koffs) for Extended Fusobody Example #5, compared to a reference CD47-Fc molecule (Example #9) are shown in Table 5B. The BiaCORE binding for these molecules are shown in FIGS. 2A and 2B respectively. The results show that the Extended Fusobody #5 has a higher avidity for SIRPalpha (based on an improved Koff or kd1). This finding is also reflected in the results listed in Table 5A, where the Extended Fusobodies show up to 200 fold improved IC₅₀ values compared to the reference CD47-Fc molecule.

TABLE 5B

| Exam- | | | | | |
|---|---|---|---|---|---|
| ple # | Description | kd1 (1/s) | KD1 (M) | kd2 (1/s) | KD2 (M) |
| #9 | CD47-Fc | 0.1092 | 9.39E−07 | 0.003399 | 1.29E−05 |
| #5 | CD47-anti- | 0.005089 | 5.35E−08 | 0.009904 | 4.11E−07 |
| | TNF-alpha | | | | |
| | Extended | | | | |
| | Fusobody | | | | |

### 4.2 Inhibition of Cytokine Release

The concentration (IC₅₀) at which inhibition of TNFalpha release occurs from Staphylococcus aureus Cowan 1 particles stimulated human monocyte-derived dendritic cells is presented in Table 6. The results demonstrate that CD47 Extended Fusobodies are functionally active to block dendritic cell activation in pM potencies. These data demonstrate that the function of CD47 domains is retained in both monospecific and bispecific Extended Fusobody scaffolds.

TABLE 6

| | | | Binding mode; | | | |
|---|---|---|---|---|---|---|
| Example | | | Valency of CD47 | | | |
| # | Format | Remark | region | IC50 nM | STDEV | N |
| 1 | CD47-Fc | Divalent CD47 Fc Reference | Monospecific; | 0.038 | 0.004 | 2 |
| | | molecule | divalent | | | |
| 2 | Non-Extended | huCD47C15G-human IgG1 LALA- | Monospecific; | 0.058 | 0.033 | 5 |
| | Fusobody | hkappa | tetravalent | | | |
| 3 | Non-Extended | huCD47 wild type-2GS linker- | Monospecific; | 0.059 | 0.065 | 25 |
| | Fusobody | human IgG1LALA-hkappa | tetravalent | | | |
| 4 | Extended Fusobody- | huCD47-1GS truncated-CH1-CH1- | Monospecific; | 0.081 | 0.031 | 4 |
| | two CH1/CL | CH2-CH3 from human IgG1 LALA | tetravalent | | | |
| | domains | | | | | |
| 5 | Extended Fusobody | huCD47 wild type-G4S-anti TNF | Bispecific; | 0.046 | 0.031 | 4 |
| | | alpha-human IgG1wt-hkappa | tetravalent | | | |
| | | Extended Fusobody having 1GS | | | | |
| | | linker | | | | |
| 6 | Extended Fusobody | huCD47 wild type-G4SG4S-anti | Bispecific; | 0.045 | 0.014 | 4 |
| | | TNF alpha-human IgG1wt-hkappa | tetravalent | | | |
| | | Extended Fusobody having 2GS | | | | |
| | | linker | | | | |
| 7 | Extended Fusobody | huCD47 wild type-G4SG4S-anti | Bispecific; | 0.053 | 0.052 | 3 |
| | | CSA-human IgG1 LALA-hkappa | tetravalent | | | |
| | | Extended Fusobody with CD47 and | | | | |
| | | cyclosporin A specificity | | | | |
| 8 | Monoclonal | anti-TNFalpha IgG1 wild type | monospecific; | 0.017 | 0.012 | 5 |
| | antibody | | bivalent | | | |

### 4.3 Binding to TNF Alpha

FIG. 3 shows that those Extended Fusobodies having specificity for both CD47 and TNFalpha (Example #5 and #6) can bind TNFalpha despite modifications introduced into the variable domains of the underlying scaffolding antibody, in this case the introduction of a linker to fuse the CD47 domains to the VH/VL of the anti-TNFalpha antibody. In contrast, a monospecific non-Extended Fusobody having CD47 specificity (Example #2) did not bind to immobilized TNFalpha. These data show that a primary antigen of 75 KDa such as TNFalpha can still be bound efficiently by CD47-TNFalpha-Extended Fusobodies containing different linker lengths. Moreover, binding to the antigen is feasible despite antigen immobilization onto a plastic surface. Other experiments have shown that soluble antigen (TNFα) can also be bound and be neutralized by CD47-TNFα Fusobodies in which the CD47 domains are simultaneously occupied by SIRPalpha (data not shown). Collectively these data confirm the mutispecific binding capability of the Extended Fusobodies of the invention.

### Useful Amino Acid and Nucleotide Sequences for Practicing the Invention

TABLE 7A
Brief description of useful amino acid and nucleotide
sequences for practicing the invention.

| SEQ ID NO: | Description of the sequence |
|---|---|
| 1 | Full length human SIRPalpha amino acid sequence (including signal |
| | sequence amino acids 1-30 (GenBank: CAC12723) |
| 2 | Full length human CD47 amino acid sequence (including signal sequence |
| | (Q08722) amino acids 1-18) |
| 3 | Extracellular Domain (ECD) of human CD47 amino acid sequence (without |
| | signal sequence) |
| 4 | Other possible ECD region of human CD47 amino acid sequence (without |
| | signal sequence) |
| 5 | CD47 extracellular domain variant with C15G mutation |
| 6 | Fc region amino acid sequence (CH2-CH3 derived from human IgG1) |
| 7 | Full length amino acid sequence of Example #1 reference CD47-Fc molecule |
| | monomer |
| 8 | G4S linker amino acid sequence |
| 9 | G4S G4S dual linker amino acid sequence |
| 10 | C_{H}1 region of heavy chain of reference Fusobody #2 and #3 and Extended |
| | Fusobodies #4, #5, #6, and #7. |
| 11 | Fc region amino acid sequence of reference Fusobody #2 and #3 and |
| | Extended Fusobodies #4, #5, #6, and #7 (CH2-CH3 derived from IgG1 with |
| | L234A L235A Fc silencing mutation) |
| 12 | Heavy chain constant region of reference Fusobody #2 and #3 and Extended |
| | Fusobodies #4, #5, #6, and #7 (CH1, CH2 and CH3) |
| 13 | C_{L} region of light chain of reference Fusobody #2 and #3 and Extended |
| | Fusobodies #4, #5, #6, and #7 (human, kappa) |
| 14 | Reference Fusobody #2 full length heavy chain of (comprising CD47 C15G |
| | variant) |
| 15 | Full length light chain of reference Fusobody #2(comprising CD47 C15G |
| | variant) |
| 16 | Full length heavy chain of reference Fusobody #3 (comprising wt CD47 |
| | sequence and two G4S linker sequences) |
| 17 | Full length light chain of reference Fusobody #3 (comprising wt CD47 |
| | sequence and two G4S linker sequences) |
| 18 | Extended Fusobody #4 full length heavy chain sequence (monospecific, |
| | comprising dual CH1 sequences and a G4S sequence linking the N-terminal |
| | CH1 sequence to the CD47 sequence) |
| 19 | Extended Fusobody #4 full length light chain sequence (monospecific, |
| | comprising dual CL sequences and a G4S sequence linking the N-terminal |
| | CL sequence to the CD47 sequence) |
| 20 | Extended Fusobody #5 full length heavy chain sequence (bispecificity for |
| | TNFalpha and SIRPalpha), comprising TNFalpha VH sequence fused to |
| | CH1, CH2 and CH3 sequences derived from IgG1 and a G4S sequence |
| | linking the TNFalpha VH sequence to the CD47 sequence) |
| 21 | Extended Fusobody #5 full length light chain sequence (bispecificity for |
| | TNFalpha and SIRPalpha), comprising TNFalpha VL sequence fused to CL, |
| | human, kappa, and a G4S sequence linking the N-terminal CL sequence to |
| | the CD47 sequence) |
| 22 | Extended Fusobody #6 full length heavy chain sequence (bispecificity for |
| | TNFalpha and SIRPalpha), comprising TNFalpha VH sequence fused to |
| | CH1, CH2 and CH3 sequences derived from IgG1 and a dual G4S sequence |
| | linking the TNFalpha VH sequence to the CD47 sequence) |
| 23 | Extended Fusobody #6 full length light chain sequence (bispecificity for |
| | TNFalpha and SIRPalpha), comprising TNFalpha VL sequence fused to CL, |
| | human, kappa, and a dual G4S sequence linking the N-terminal CL |
| | sequence to the CD47 sequence) |
| 24 | Heavy chain antibody sequence of Extended Fusobody #5 and #6 |
| | (comprising TNFalpha VH sequence fused to CH1, CH2 and CH3 |
| | sequences derived from IgG1) |
| 25 | Light chain antibody sequence of Extended Fusobody #5 and #6 (comprising |
| | TNFalpha VL sequence fused to human, kappa CL sequence) |
| 26 | VH sequence of Extended Fusobody #5 and #6 (specificity for TNFalpha) |
| | and TNFalpha reference antibody #8 |
| 27 | HCDR1 of Extended Fusobody #5 and #6 and TNFalpha reference antibody #8 |
| 28 | HCDR2 of Extended Fusobody #5 and #6 and TNFalpha reference antibody #8 |
| 29 | HCDR3 of Extended Fusobody #5 and #6 and TNFalpha reference antibody #8 |
| 30 | VL sequence of Extended Fusobody #5 (specificity for TNFalpha) and |
| | TNFalpha reference antibody |
| 31 | LCDR1 of Extended Fusobody #5 and #6 and TNFalpha reference antibody #8 |
| 32 | LCDR2 of Extended Fusobody #5 and #6 and TNFalpha reference antibody #8 |
| 33 | LCDR3 of Extended Fusobody #5 and #6 and TNFalpha reference antibody #8 |
| 34 | CD47/VH sequence of Extended Fusobody #5 |
| 35 | CD47/VL sequence of Extended Fusobody #5 |
| 36 | CD47/VH sequence of Extended Fusobody #6 |
| 37 | CD47/VL sequence of Extended Fusobody #6 |
| 38 | Full length heavy chain of TNFalpha reference antibody |
| 39 | Full length light chain of TNFalpha reference antibody |
| 40 | Extended Fusobody #7 full length heavy chain sequence (bispecificity for |
| | cyclosporin A and SIRPalpha), comprising cyclosporin A VH sequence fused |
| | to CH1, CH2 and CH3 sequences derived from IgG1 and a dual G4S |
| | sequence linking the cyclosporin A VH sequence to the CD47 sequence |
| 41 | Extended Fusobody #7 full length light chain sequence (bispecificity for |
| | cyclosporin A and SIRPalpha), comprising cyclosporin A VL sequence fused |
| | to CL, human, kappa, and a dual G4S sequence linking the N-terminal CL |
| | sequence to the CD47 sequence) |
| 42 | Heavy chain antibody sequence of Extended Fusobody #7 (comprising |
| | cyclosporin A VH sequence fused to CH1, CH2 and CH3 sequences, IgG1 |
| | LALA) |
| 43 | Light chain antibody sequence of Extended Fusobody #7 (comprising |
| | cyclosporin A VL sequence fused to human, kappa CL sequence) |
| 44 | VH sequence of Extended Fusobody #7 (specificity for cyclosporin A) |
| 45 | HCDR1 of Extended Fusobody #7 |
| 46 | HCDR2 of Extended Fusobody #7 |
| 47 | HCDR3 of Extended Fusobody #7 |
| 48 | VL sequence of Extended Fusobody #7 (specificity for cyclosporin A) |
| 49 | LCDR1 of Extended Fusobody #7 |
| 50 | LCDR2 of Extended Fusobody #7 |
| 51 | LCDR3 of Extended Fusobody #7 |
| 52 | CD47/VH sequence of Extended Fusobody #7 |
| 53 | CD47/VL sequence of Extended Fusobody #7 |
| 54 | C_{H}1 region of heavy chain of TNFalpha reference antibody #8 |
| 55 | C_{L} region of light chain of TNFalpha reference antibody #8 |
| 56 | Fc region amino acid sequence of TNFalpha reference antibody #8 |
| 57 | CD47 extracellular domain truncated variant (shortened C-terminal part) |
| 58 | Nucleic acid sequence of SEQ ID NO: 1 |
| 59 | Nucleic acid sequence of SEQ ID NO: 2 |
| 60 | Nucleic acid sequence of SEQ ID NO: 3 |
| 61 | Nucleic acid sequence of SEQ ID NO: 4 |
| 62 | Nucleic acid sequence of SEQ ID NO: 5 |
| 63 | Nucleic acid sequence of SEQ ID NO: 6 |
| 64 | Nucleic acid sequence of SEQ ID NO: 7 |
| 65 | Nucleic acid sequence of SEQ ID NO: 8 |
| 66 | Nucleic acid sequence of SEQ ID NO: 9, for Example #2 and #3 |
| 67 | Nucleic acid sequence of SEQ ID NO: 10 |
| 68 | Nucleic acid sequence of SEQ ID NO: 11 |
| 69 | Nucleic acid sequence of SEQ ID NO: 12 |
| 70 | Nucleic acid sequence of SEQ ID NO: 13 |
| 71 | Nucleic acid sequence of SEQ ID NO: 14 |
| 72 | Nucleic acid sequence of SEQ ID NO: 15 |
| 73 | Nucleic acid sequence of SEQ ID NO: 16 |
| 74 | Nucleic acid sequence of SEQ ID NO: 17 |
| 75 | Nucleic acid sequence of SEQ ID NO: 18 |
| 76 | Nucleic acid sequence of SEQ ID NO: 19 |
| 77 | Nucleic acid sequence of SEQ ID NO: 20 |
| 78 | Nucleic acid sequence of SEQ ID NO: 21 |
| 79 | Nucleic acid sequence of SEQ ID NO: 22 |
| 80 | Nucleic acid sequence of SEQ ID NO: 23 |
| 81 | Nucleic acid sequence of SEQ ID NO: 24 |
| 82 | Nucleic acid sequence of SEQ ID NO: 25 |
| 83 | Nucleic acid sequence of SEQ ID NO: 26, encoding Example #6 |
| 84 | Nucleic acid sequence of SEQ ID NO: 27 |
| 85 | Nucleic acid sequence of SEQ ID NO: 28 |
| 86 | Nucleic acid sequence of SEQ ID NO: 29 |
| 87 | Nucleic acid sequence of SEQ ID NO: 30 |
| 88 | Nucleic acid sequence of SEQ ID NO: 31 |
| 89 | Nucleic acid sequence of SEQ ID NO: 32 |
| 90 | Nucleic acid sequence of SEQ ID NO: 33 |
| 91 | Nucleic acid sequence of SEQ ID NO: 34 |
| 92 | Nucleic acid sequence of SEQ ID NO: 35 |
| 93 | Nucleic acid sequence of SEQ ID NO: 36 |
| 94 | Nucleic acid sequence of SEQ ID NO: 37 |
| 95 | Nucleic acid sequence of SEQ ID NO: 38 |
| 96 | Nucleic acid sequence of SEQ ID NO: 39 |
| 97 | Nucleic acid sequence of SEQ ID NO: 40 |
| 98 | Nucleic acid sequence of SEQ ID NO: 41 |
| 99 | Nucleic acid sequence of SEQ ID NO: 42 |
| 100 | Nucleic acid sequence of SEQ ID NO: 43 |
| 101 | Nucleic acid sequence of SEQ ID NO: 44 |
| 102 | Nucleic acid sequence of SEQ ID NO: 45 |
| 103 | Nucleic acid sequence of SEQ ID NO: 46 |
| 104 | Nucleic acid sequence of SEQ ID NO: 47 |
| 105 | Nucleic acid sequence of SEQ ID NO: 48 |
| 106 | Nucleic acid sequence of SEQ ID NO: 49 |
| 107 | Nucleic acid sequence of SEQ ID NO: 50 |
| 108 | Nucleic acid sequence of SEQ ID NO: 51 |
| 109 | Nucleic acid sequence of SEQ ID NO: 52 |
| 110 | Nucleic acid sequence of SEQ ID NO: 53 |
| 111 | Nucleic acid sequence of SEQ ID NO: 54 |
| 112 | Nucleic acid sequence of SEQ ID NO: 55 |
| 113 | Nucleic acid sequence of SEQ ID NO: 56 |
| 114 | Nucleic acid sequence of SEQ ID NO: 57 |
| 115 | Amino acid sequence of SIRPgamma NP_061026.2 |
| 116 | Fc region amino acid sequence (CH2-CH3 derived from human IgG1 bearing |
| | N297A mutation) |
| 117 | Full length amino acid sequence of Example #9 reference CD47-Fc molecule |
| | monomer |
| 118 | Nucleic acid sequence of SEQ ID NO: 116 |
| 119 | Nucleic acid sequence of SEQ ID NO: 117 |
| 120 | Amino acid sequence linker example #4 (seq18) |
| 121 | Nucleic acid sequence of SEQ ID NO: 120 |
| 122 | Amino acid sequence for Fc region of IgG1 wild type |
| 123 | Nucleic acid sequence of SEQ ID NO: 122 |
| 124 | Alternative nucleic acid sequence of SEQ ID NO: 10, used with Example #2 |
| | and #3 |
| 125 | Alternative nucleic acid sequence of SEQ ID NO: 9, used with Example #4 |
| 126 | Alternative nucleic acid sequence of SEQ ID NO: 9, used with Example #6 |
| | and #7 |
| 127 | Nucleic acid sequence of SEQ ID NO: 26, used with Example #5 |

TABLE 7B
Sequence listing

| SEQ ID | |
|---|---|
| NO: | AMINO ACID OR NUCLEOTIDE SEQUENCE |
| 1 | MEPAGPAPGRLGPLLCLLLAASCAWSGVAGEEELQVIQPDKSVLVAAGETATLRC |
| | TATSLIPVGPIQWFRGAGPGRELIYNQKEGHFPRVTTVSDLTKRNNMDFSIRIGNIT |
| | PADAGTYYCVKFRKGSPDDVEFKSGAGTELSVRAKPSAPVVSGPAARATPQHTV |
| | SFTCESHGFSPRDITLKWFKNGNELSDFQTNVDPVGESVSYSIHSTAKVVLTRED |
| | VHSQVICEVAHVTLQGDPLRGTANLSETIRVPPTLEVTQQPVRAENQVNVTCQVR |
| | KFYPQRLQLTWLENGNVSRTETASTVTENKDGTYNWMSWLLVNVSAHRDDVKLT |
| | CQVEHDGQPAVSKSHDLKVSAHPKEQGSNTAAENTGSNERNIYIVVGVVCTLLVA |
| | LLMAALYLVRIRQKKAQGSTSSTRLHEPEKNAREITQDTNDITYADLNLPKGKKPA |
| | PQAAEPNNHTEYASIQTSPQPASEDTLTYADLDMVHLNRTPKQPAPKPEPSFSEY |
| | ASVQVPRK |
| 2 | MWPLVAALLLGSACCGSAQLLFNKTKSVEFTFCNDTVVIPCFVTNMEAQNTTEVY |
| | VKWKFKGRDIYTFDGALNKSTVPTDFSSAKIEVSQLLKGDASLKMDKSDAVSHTG |
| | NYTCEVTELTREGETIIELKYRVVSWFSPNENILIVIFPIFAILLFWGQFGIKTLKYRS |
| | GGMDEKTIALLVAGLVITVIVIVGAILFVPGEYSLKNATGLGLIVTSTGILILLHYYVFS |
| | TAIGLTSFVIAILVIQVIAYILAVVGLSLCIAACIPMHGPLLISGLSILALAQLLGLVYMKF |
| | VASNQKTIQPPRKAVEEPLNAFKESKGMMNDE |
| 3 | QLLFNKTKSVEFTFCNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVSWFSPNE |
| 4 | QLLFNKTKSVEFTFCNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVSWFSPNEN |
| 5 | QLLFNKTKSVEFTFGNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVSWFSPNEN |
| 6 | LEPKSCDKTHTCPPCPAPEAAGGPSVFLFPPKPKDTLMISRTPEVTCVVVDVSHE |
| | DPEVKFNVVYVDGVEVHNAKTKPREEQYNSTYRVVSVLTVLHQDWLNGKEYKCK |
| | VSNKALPAPIEKTISKAKGQPREPQVYTLPPSREEMTKNQVSLTCLVKGFYPSDIA |
| | VEWESNGQPENNYKTTPPVLDSDGSFFLYSKLTVDKSRWQQGNVFSCSVMHEA |
| | LHNHYTQKSLSLSPGK |
| 7 | QLLFNKTKSV EFTFCNDTW IPCFVTNMEA QNTTEVYVKW |
| | KFKGRDIYTFDGALNKSTVP TDFSSAKIEV SQLLKGDASL KMDKSDAVSH |
| | TGNYTCEVTELTREGETIIE LKYRVVSWFS PNENLEPKSC DKTHTCPPCP |
| | APEAAGGPSVFLFPPKPKDT LMISRTPEVT CVVVDVSHED PEVKFNWYVD |
| | GVEVHNAKTKPREEQYNSTY RVVSVLTVLH QDWLNGKEYK CKVSNKALPA |
| | PIEKTISKAKGQPREPQVYT LPPSREEMTK NQVSLTCLVK GFYPSDIAVE |
| | WESNGQPENNYKTTPPVLDS DGSFFLYSKL TVDKSRWQQG NVFSCSVMHE |
| | ALHNHYTQKSLSLSPGK |
| 8 | GGGGS |
| 9 | GGGGSGGGGS |
| 10 | SASTKGPSVFPLAPSSKSTSGGTAALGCLVKDYFPEPVTVSWNSGALTSGVHTFP |
| | AVLQSSGLYSLSSVVTVPSSSLGTQTYICNVNHKPSNTKVDKRV |
| 11 | EPKSCDKTHTCPPCPAPEAAGGPSVFLFPPKPKDTLMISRTPEVTCVVVDVSHED |
| | PEVKFNWYVDGVEVHNAKTKPREEQYNSTYRVVSVLTVLHQDWLNGKEYKCKVS |
| | NKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALH |
| | NHYTQKSLSLSPGK |
| 12 | SASTKGPSVFPLAPSSKSTSGGTAALGCLVKDYFPEPVTVSWNSGALTSGVHTFP |
| | AVLQSSGLYSLSSVVTVPSSSLGTQTYICNVNHKPSNTKVDKRV |
| | EPKSCDKTHTCPPCPAPEAAGGPSVFLFPPKPKDTLMISRTPEVTCVVVDVSHED |
| | PEVKFNWYVDGVEVHNAKTKPREEQYNSTYRVVSVLTVLHQDWLNGKEYKCKVS |
| | NKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALH |
| | NHYTQKSLSLSPGK |
| 13 | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| 14 | QLLFNKTKSVEFTFGNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVSWFSPNENGGGGSGGGGSSASTKGPSVFPLAPSSKSTSGGTAALGCLVKD |
| | YFPEPVTVSWNSGALTSGVHTFPAVLQSSGLYSLSSVVTVPSSSLGTQTYICNVN |
| | HKPSNTKVDKRVEPKSCDKTHTCPPCPAPEAAGGPSVFLFPPKPKDTLMISRTPE |
| | VTCVVVDVSHEDPEVKFNWYVDGVEVHNAKTKPREEQYNSTYRVVSVLTVLHQD |
| | WLNGKEYKCKVSNKALPAPIEKTISKAKGQPREPQVYTLPPSREEMTKNQVSLTC |
| | LVKGFYPSDIAVEWESNGQPENNYKTTPPVLDSDGSFFLYSKLTVDKSRWQQGN |
| | VFSCSVMHEALHNHYTQKSLSLSPGK |
| 15 | QLLFNKTKSVEFTFGNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVSWFSPNENGGGGSGGGGSRTVAAPSVFIFPPSDEQLKSGTASVVCLLNNFY |
| | PREAKVQWKVDNALQSGNSQESVTEQDSKDSTYSLSSTLTLSKADYEKHKVYAC |
| | EVTHQGLSSPVTKSFNRGEC |
| 16 | QLLFNKTKSVEFTFCNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVSWFSPNENGGGGSGGGGSSASTKGPSVFPLAPSSKSTSGGTAALGCLVKD |
| | YFPEPVTVSWNSGALTSGVHTFPAVLQSSGLYSLSSVVTVPSSSLGTQTYICNVN |
| | HKPSNTKVDKRVEPKSCDKTHTCPPCPAPEAAGGPSVFLFPPKPKDTLMISRTPE |
| | VTCVVVDVSHEDPEVKFNWYVDGVEVHNAKTKPREEQYNSTYRVVSVLTVLHQD |
| | WLNGKEYKCKVSNKALPAPIEKTISKAKGQPREPQVYTLPPSREEMTKNQVSLTC |
| | LVKGFYPSDIAVEWESNGQPENNYKTTPPVLDSDGSFFLYSKLTVDKSRWQQGN |
| | VFSCSVMHEALHNHYTQKSLSLSPGK |
| 17 | QLLFNKTKSVEFTFCNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVSWFSPNENGGGGSGGGGSRTVAAPSVFIFPPSDEQLKSGTASVVCLLNNFY |
| | PREAKVQWKVDNALQSGNSQESVTEQDSKDSTYSLSSTLTLSKADYEKHKVYAC |
| | EVTHQGLSSPVTKSFNRGEC |
| 18 | QLLFNKTKSVEFTFCNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVSGGGGSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKRVEP |
| | KSCGGGGSGGGGSSASTKGPSVFPLAPSSKSTSGGTAALGCLVKDYFPEPVTVS |
| | WNSGALTSGVHTFPAVLQSSGLYSLSSVVTVPSSSLGTQTYICNVNHKPSNTKVD |
| | KRVEPKSCDKTHTCPPCPAPEAAGGPSVFLFPPKPKDTLMISRTPEVTCVVVDVS |
| | HEDPEVKFNVVYVDGVEVHNAKTKPREEQYNSTYRVVSVLTVLHQDWLNGKEYK |
| | CKVSNKALPAPIEKTISKAKGQPREPQVYTLPPSREEMTKNQVSLTCLVKGFYPSD |
| | IAVEWESNGQPENNYKTTPPVLDSDGSFFLYSKLTVDKSRWQQGNVFSCSVMHE |
| | ALHNHYTQKSLSLSPGK |
| 19 | QLLFNKTKSVEFTFCNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVSGGGGSRTVAAPSVFIFPPSDEQLKSGTASVVCLLNNFYPREAKVQWKVDN |
| | ALQSGNSQESVTEQDSKDSTYSLSSTLTLSKADYEKHKVYACEVTHQGLSSPVTK |
| | SFNRGECGGGGSGGGGSRTVAAPSVFIFPPSDEQLKSGTASVVCLLNNFYPREA |
| | KVQWKVDNALQSGNSQESVTEQDSKDSTYSLSSTLTLSKADYEKHKVYACEVTH |
| | QGLSSPVTKSFNRGEC |
| 20 | QLLFNKTKSVEFTFCNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVSWFSPNENEVQLVESGGGLVQPGRSLRLSCAASGFTFDDYAMHWVRQAPG |
| | KGLEVVVSAITWNSGHIDYADSVEGRFTISRDNAKNSLYLQMNSLRAEDTAVYYCA |
| | KVSYLSTASSLDYWGQGTLVTVSSASTKGPSVFPLAPSSKSTSGGTAALGCLVKD |
| | YFPEPVTVSWNSGALTSGVHTFPAVLQSSGLYSLSSVVTVPSSSLGTQTYICNVN |
| | HKPSNTKVDKRVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLTVLHQD |
| | WLNGKEYKCKVSNKALPAPIEKTISKAKGQPREPQVYTLPPSREEMTKNQVSLTC |
| | LVKGFYPSDIAVEWESNGQPENNYKTTPPVLDSDGSFFLYSKLTVDKSRWQQGN |
| | VFSCSVMHEALHNHYTQKSLSLSPGK |
| 21 | QLLFNKTKSVEFTFCNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVSWFSPNENDIQMTQSPSSLSASVGDRVTITCRASQGIRNYLAVVYQQKPGKA |
| | PKLLIYAASTLQSGVPSRFSGSGSGTDFTLTISSLQPEDVATYYCQRYNRAPYTFG |
| | QGTKVEIKRTVAAPSVFIFPPSDEQLKSGTASVVCLLNNFYPREAKVQWKVDNAL |
| | QSGNSQESVTEQDSKDSTYSLSSTLTLSKADYEKHKVYACEVTHQGLSSPVTKSF |
| | NRGEC |
| 22 | QLLFNKTKSVEFTFCNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVSWFSPNENGGGGSGGGGSEVQLVESGGGLVQPGRSLRLSCAASGFTFDDY |
| | AMHWVRQAPGKGLEVVVSAITWNSGHIDYADSVEGRFTISRDNAKNSLYLQMNSL |
| | RAEDTAVYYCAKVSYLSTASSLDYWGQGTLVTVSSASTKGPSVFPLAPSSKSTSG |
| | GTAALGCLVKDYFPEPVTVSWNSGALTSGVHTFPAVLQSSGLYSLSSVVTVPSSS |
| | LGTQTYICNVNHKPSNTKVDKRVEPKSCDKTHTCPPCPAPELLGGPSVFLFPPKP |
| | KDTLMISRTPEVTCVVVDVSHEDPEVKFNVVYVDGVEVHNAKTKPREEQYNSTYR |
| | VVSVLTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| 23 | QLLFNKTKSVEFTFCNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVSWFSPNENGGGGSGGGGSDIQMTQSPSSLSASVGDRVTITCRASQGIRNYL |
| | AWYQQKPGKAPKLLIYAASTLQSGVPSRFSGSGSGTDFTLTISSLQPEDVATYYC |
| | QRYNRAPYTFGQGTKVEIKRTVAAPSVFIFPPSDEQLKSGTASVVCLLNNFYPREA |
| | KVQWKVDNALQSGNSQESVTEQDSKDSTYSLSSTLTLSKADYEKHKVYACEVTH |
| | QGLSSPVTKSFNRGEC |
| 24 | EVQLVESGGGLVQPGRSLRLSCAASGFTFDDYAMHWVRQAPGKGLEWVSAITW |
| | NSGHIDYADSVEGRFTISRDNAKNSLYLQMNSLRAEDTAVYYCAKVSYLSTASSLD |
| | YWGQGTLVTVSSASTKGPSVFPLAPSSKSTSGGTAALGCLVKDYFPEPVTVSWN |
| | SGALTSGVHTFPAVLQSSGLYSLSSWTVPSSSLGTQTYICNVNHKPSNTKVDKR |
| | VEPKSCDKTHTCPPCPAPELLGGPSVFLFPPKPKDTLMISRTPEVTCVVVDVSHE |
| | DPEVKFNVVYVDGVEVHNAKTKPREEQYNSTYRVVSVLTVLHQDWLNGKEYKCK |
| | VSNKALPAPIEKTISKAKGQPREPQVYTLPPSREEMTKNQVSLTCLVKGFYPSDIA |
| | VEWESNGQPENNYKTTPPVLDSDGSFFLYSKLTVDKSRWQQGNVFSCSVMHEA |
| | LHNHYTQKSLSLSPGK |
| 25 | DIQMTQSPSSLSASVGDRVTITCRASQGIRNYLAWYQQKPGKAPKLLIYAASTLQS |
| | GVPSRFSGSGSGTDFTLTISSLQPEDVATYYCQRYNRAPYTFGQGTKVEIKRTVA |
| | APSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC |
| 26 | EVQLVESGGGLVQPGRSLRLSCAASGFTFDDYAMHWVRQAPGKGLEWVSAITW |
| | NSGHIDYADSVEGRFTISRDNAKNSLYLQMNSLRAEDTAVYYCAKVSYLSTASSLD |
| | YWGQGTLVTVS |
| 27 | DYAMH |
| 28 | AITWNSGHIDYADSVEG |
| 29 | VSYLSTASSLDY |
| 30 | DIQMTQSPSSLSASVGDRVTITCRASQGIRNYLAVVYQQKPGKAPKLLIYAASTLQS |
| | GVPSRFSGSGSGTDFTLTISSLQPEDVATYYCQRYNRAPYTFGQGTKVEIK |
| 31 | RASQGIRNYLA |
| 32 | AASTLQS |
| 33 | QRYNRAPYT |
| 34 | QLLFNKTKSVEFTFCNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVSWFSPNENEVQLVESGGGLVQPGRSLRLSCAASGFTFDDYAMHWVRQAPG |
| | KGLEWVSAITWNSGHIDYADSVEGRFTISRDNAKNSLYLQMNSLRAEDTAVYYCA |
| | KVSYLSTASSLDYWGQGTLVTVSS |
| 35 | QLLFNKTKSVEFTFCNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVSWFSPNENDIQMTQSPSSLSASVGDRVTITCRASQGIRNYLAWYQQKPGKA |
| | PKLLIYAASTLQSGVPSRFSGSGSGTDFTLTISSLQPEDVATYYCQRYNRAPYTFG |
| | QGTKVEIK |
| 36 | QLLFNKTKSVEFTFCNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVSWFSPNENGGGGSGGGGSEVQLVESGGGLVQPGRSLRLSCAASGFTFDDY |
| | AMHWVRQAPGKGLEWVSAITWNSGHIDYADSVEGRFTISRDNAKNSLYLQMNSL |
| | RAEDTAVYYCAKVSYLSTASSLDYWGQGTLVTVSS |
| 37 | QLLFNKTKSVEFTFCNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVSWFSPNENGGGGSGGGGSDIQMTQSPSSLSASVGDRVTITCRASQGIRNYL |
| | AWYQQKPGKAPKLLIYAASTLQSGVPSRFSGSGSGTDFTLTISSLQPEDVATYYC |
| | QRYNRAPYTFGQGTKVEIK |
| 38 | EVQLVESGGGLVQPGRSLRLSCAASGFTFDDYAMHWVRQAPGKGLEWVSAITW |
| | NSGHIDYADSVEGRFTISRDNAKNSLYLQMNSLRAEDTAVYYCAKVSYLSTASSLD |
| | YWGQGTLVTVSSASTKGPSVFPLAPSSKSTSGGTAALGCLVKDYFPEPVTVSWN |
| | SGALTSGVHTFPAVLQSSGLYSLSSWTVPSSSLGTQTYICNVNHKPSNTKVDKKV |
| | EPKSCDKTHTCPPCPAPELLGGPSVFLFPPKPKDTLMISRTPEVTCVVVDVSHED |
| | PEVKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVE |
| | WESNGQPENNYKTTPPVLDSDGSFFLYSKLTVDKSRWQQGNVFSCSVMHEALH |
| | NHYTQKSLSLSPGK |
| 39 | DIQMTQSPSSLSASVGDRVTITCRASQGIRNYLAWYQQKPGKAPKLLIYAASTLQS |
| | GVPSRFSGSGSGTDFTLTISSLQPEDVATYYCQRYNRAPYTFGQGTKVEIKRTVA |
| | APSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC |
| 40 | QLLFNKTKSVEFTFCNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVSWFSPNENGGGGSGGGGSEVQLEQSGPVLVKPGTSMKISCKTSGYSFTGY |
| | TMSWVRQSHGKSLEWIGLIIPSNGGTNYNQKFKDKASLTVDKSSSTAYMELLSLT |
| | SEDSAVYYCARPSYYGSRNYYAMDYWGQGTSVTVSSASTKGPSVFPLAPSSKST |
| | SGGTAALGCLVKDYFPEPVTVSWNSGALTSGVHTFPAVLQSSGLYSLSSVVTVPS |
| | SSLGTQTYICNVNHKPSNTKVDKRVEPKSCDKTHTCPPCPAPEAAGGPSVFLFPP |
| | KPKDTLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVYTLPPS |
| | REEMTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| 41 | QLLFNKTKSVEFTFCNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVSWFSPNENGGGGSGGGGSDIVLTQSPASLAVSLGQRATISCRASESVDNSG |
| | FSFMNWFQQKPGQPPKLLIYAASNQGSGVPARFSGSGSETDFSLNIHPMEEDDT |
| | AVYFCQQSKEVPWTFGGGTKLEIKRTVAAPSVFIFPPSDEQLKSGTASVVCLLNNF |
| | YPREAKVQWKVDNALQSGNSQESVTEQDSKDSTYSLSSTLTLSKADYEKHKVYA |
| | CEVTHQGLSSPVTKSFNRGEC |
| 42 | EVQLEQSGPVLVKPGTSMKISCKTSGYSFTGYTMSWVRQSHGKSLEWIGLIIPSN |
| | GGTNYNQKFKDKASLTVDKSSSTAYMELLSLTSEDSAVYYCARPSYYGSRNYYA |
| | MDYWGQGTSVTVSSASTKGPSVFPLAPSSKSTSGGTAALGCLVKDYFPEPVTVS |
| | WNSGALTSGVHTFPAVLQSSGLYSLSSVVTVPSSSLGTQTYICNVNHKPSNTKVD |
| | KRVEPKSCDKTHTCPPCPAPEAAGGPSVFLFPPKPKDTLMISRTPEVTCVVVDVS |
| | HEDPEVKFNWYVDGVEVHNAKTKPREEQYNSTYRVVSVLTVLHQDWLNGKEYK |
| | CKVSNKALPAPIEKTISKAKGQPREPQVYTLPPSREEMTKNQVSLTCLVKGFYPSD |
| | IAVEWESNGQPENNYKTTPPVLDSDGSFFLYSKLTVDKSRWQQGNVFSCSVMHE |
| | ALHNHYTQKSLSLSPGK |
| 43 | DIVLTQSPASLAVSLGQRATISCRASESVDNSGFSFMNWFQQKPGQPPKLLIYAAS |
| | NQGSGVPARFSGSGSETDFSLNIHPMEEDDTAVYFCQQSKEVPWTFGGGTKLEI |
| | KRTVAAPSVFIFPPSDEQLKSGTASVVCLLNNFYPREAKVQWKVDNALQSGNSQE |
| | SVTEQDSKDSTYSLSSTLTLSKADYEKHKVYACEVTHQGLSSPVTKSFNRGEC |
| 44 | EVQLEQSGPVLVKPGTSMKISCKTSGYSFTGYTMSWVRQSHGKSLEWIGLIIPSN |
| | GGTNYNQKFKDKASLTVDKSSSTAYMELLSLTSEDSAVYYCARPSYYGSRNYYA |
| | MDYWGQGTSVTVS |
| 45 | GYTMS |
| 46 | LIIPSNGGTNYNQKFKD |
| 47 | PSYYGSRNYYAMDY |
| 48 | DIVLTQSPASLAVSLGQRATISCRASESVDNSGFSFMNWFQQKPGQPPKLLIYAAS |
| | NQGSGVPARFSGSGSETDFSLNIHPMEEDDTAVYFCQQSKEVPWTFGGGTKLEI |
| | K |
| 49 | RASESVDNSGFSFMN |
| 50 | AASNQGS |
| 51 | QQSKEVPWT |
| 52 | QLLFNKTKSVEFTFCNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVSWFSPNENGGGGSGGGGSEVQLEQSGPVLVKPGTSMKISCKTSGYSFTGY |
| | TMSWVRQSHGKSLEWIGLIIPSNGGTNYNQKFKDKASLTVDKSSSTAYMELLSLT |
| | SEDSAVYYCARPSYYGSRNYYAMDYWGQGTSVTVSS |
| 53 | QLLFNKTKSVEFTFCNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVSWFSPNENGGGGSGGGGSDIVLTQSPASLAVSLGQRATISCRASESVDNSG |
| | FSFMNWFQQKPGQPPKLLIYAASNQGSGVPARFSGSGSETDFSLNIHPMEEDDT |
| | AVYFCQQSKEVPWTFGGGTKLEIK |
| 54 | SASTKGPSVFPLAPSSKSTSGGTAALGCLVKDYFPEPVTVSWNSGALTSGVHTFP |
| | AVLQSSGLYSLSSVVTVPSSSLGTQTYICNVNHKPSNTKVDKKV |
| 55 | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| 56 | EPKSCDKTHTCPPCPAPELLGGPSVFLFPPKPKDTLMISRTPEVTCVVVDVSHED |
| | PEVKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVE |
| | WESNGQPENNYKTTPPVLDSDGSFFLYSKLTVDKSRWQQGNVFSCSVMHEALH |
| | NHYTQKSLSLSPGK |
| 57 | QLLFNKTKSVEFTFCNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVS |
| 58 | ATGGAGCCCGCCGGCCCGGCCCCCGGCCGCCTCGGGCCGCTGCTCTGCCTG |
| | CTGCTCGCCGCGTCCTGCGCCTGGTCAGGAGTGGCGGGTGAGGAGGAGCTG |
| | CAGGTGATTCAGCCTGACAAGTCCGTGTTGGTTGCAGCTGGAGAGACAGCCA |
| | CTCTGCGCTGCACTGCGACCTCTCTGATCCCTGTGGGGCCCATCCAGTGGTT |
| | CAGAGGAGCTGGACCAGGCCGGGAATTAATCTACAATCAAAAAGAAGGCCAC |
| | TTCCCCCGGGTAACAACTGTTTCAGACCTCACAAAGAGAAACAACATGGACTT |
| | TTCCATCCGCATCGGTAACATCACCCCAGCAGATGCCGGCACCTACTACTGTG |
| | TGAAGTTCCGGAAAGGGAGCCCCGATGACGTGGAGTTTAAGTCTGGAGCAGG |
| | CACTGAGCTGTCTGTGCGCGCCAAACCCTCTGCCCCCGTGGTATCGGGCCCT |
| | GCGGCGAGGGCCACACCTCAGCACACAGTGAGCTTCACCTGCGAGTCCCACG |
| | GCTTCTCACCCAGAGACATCACCCTGAAATGGTTCAAAAATGGGAATGAGCTC |
| | TCAGACTTCCAGACCAACGTGGACCCCGTAGGAGAGAGCGTGTCCTACAGCA |
| | TCCACAGCACAGCCAAGGTGGTGCTGACCCGCGAGGACGTTCACTCTCAAGT |
| | CATCTGCGAGGTGGCCCACGTCACCTTGCAGGGGGACCCTCTTCGTGGGACT |
| | GCCAACTTGTCTGAGACCATCCGAGTTCCACCCACCTTGGAGGTTACTCAACA |
| | GCCCGTGAGGGCAGAGAACCAGGTGAATGTCACCTGCCAGGTGAGGAAGTTC |
| | TACCCCCAGAGACTACAGCTGACCTGGTTGGAGAATGGAAACGTGTCCCGGA |
| | CAGAAACGGCCTCAACCGTTACAGAGAACAAGGATGGTACCTACAACTGGATG |
| | AGCTGGCTCCTGGTGAATGTATCTGCCCACAGGGATGATGTGAAGCTCACCTG |
| | CCAGGTGGAGCATGACGGGCAGCCAGCGGTCAGCAAAAGCCATGACCTGAA |
| | GGTCTCAGCCCACCCGAAGGAGCAGGGCTCAAATACCGCCGCTGAGAACACT |
| | GGATCTAATGAACGGAACATCTATATTGTGGTGGGTGTGGTGTGCACCTTGCT |
| | GGTGGCCCTACTGATGGCGGCCCTCTACCTCGTCCGAATCAGACAGAAGAAA |
| | GCCCAGGGCTCCACTTCTTCTACAAGGTTGCATGAGCCCGAGAAGAATGCCA |
| | GAGAAATAACACAGGACACAAATGATATCACATATGCAGACCTGAACCTGCCC |
| | AAGGGGAAGAAGCCTGCTCCCCAGGCTGCGGAGCCCAACAACCACACGGAG |
| | TATGCCAGCATTCAGACCAGCCCGCAGCCCGCGTCGGAGGACACCCTCACCT |
| | ATGCTGACCTGGACATGGTCCACCTCAACCGGACCCCCAAGCAGCCGGCCCC |
| | CAAGCCTGAGCCGTCCTTCTCAGAGTACGCCAGCGTCCAGGTCCCGAGGAAG |
| | TGA |
| 59 | ATGTGGCCCCTGGTAGCGGCGCTGTTGCTGGGCTCGGCGTGCTGCGGATCA |
| | GCTCAGCTACTATTTAATAAAACAAAATCTGTAGAATTCACGTTTTGTAATGACA |
| | CTGTCGTCATTCCATGCTTTGTTACTAATATGGAGGCACAAAACACTACTGAAG |
| | TATACGTAAAGTGGAAATTTAAAGGAAGAGATATTTACACCTTTGATGGAGCTC |
| | TAAACAAGTCCACTGTCCCCACTGACTTTAGTAGTGCAAAAATTGAAGTCTCAC |
| | AATTACTAAAAGGAGATGCCTCTTTGAAGATGGATAAGAGTGATGCTGTCTCAC |
| | ACACAGGAAACTACACTTGTGAAGTAACAGAATTAACCAGAGAAGGTGAAACG |
| | ATCATCGAGCTAAAATATCGTGTTGTTTCATGGTTTTCTCCAAATGAAAATATTC |
| | TTATTGTTATTTTCCCAATTTTTGCTATACTCCTGTTCTGGGGACAGTTTGGTAT |
| | TAAAACACTTAAATATAGATCCGGTGGTATGGATGAGAAAACAATTGCTTTACT |
| | TGTTGCTGGACTAGTGATCACTGTCATTGTCATTGTTGGAGCCATTCTTTTCGT |
| | CCCAGGTGAATATTCATTAAAGAATGCTACTGGCCTTGGTTTAATTGTGACTTC |
| | TACAGGGATATTAATATTACTTCACTACTATGTGTTTAGTACAGCGATTGGATTA |
| | ACCTCCTTCGTCATTGCCATATTGGTTATTCAGGTGATAGCCTATATCCTCGCT |
| | GTGGTTGGACTGAGTCTCTGTATTGCGGCGTGTATACCAATGCATGGCCCTCT |
| | TCTGATTTCAGGTTTGAGTATCTTAGCTCTAGCACAATTACTTGGACTAGTTTAT |
| | ATGAAATTTGTGGCTTCCAATCAGAAGACTATACAACCTCCTAGGAAAGCTGTA |
| | GAGGAACCCCTTAATGCATTCAAAGAATCAAAAGGAATGATGAATGATGAATAA |
| 60 | CAGCTACTATTTAATAAAACAAAATCTGTAGAATTCACGTTTTGTAATGACACTG |
| | TCGTCATTCCATGCTTTGTTACTAATATGGAGGCACAAAACACTACTGAAGTAT |
| | ACGTAAAGTGGAAATTTAAAGGAAGAGATATTTACACCTTTGATGGAGCTCTAA |
| | ACAAGTCCACTGTCCCCACTGACTTTAGTAGTGCAAAAATTGAAGTCTCACAAT |
| | TACTAAAAGGAGATGCCTCTTTGAAGATGGATAAGAGTGATGCTGTCTCACAC |
| | ACAGGAAACTACACTTGTGAAGTAACAGAATTAACCAGAGAAGGTGAAACGAT |
| | CATCGAGCTAAAATATCGTGTTGTTTCATGGTTTTCTCCAAATGAA |
| 61 | CAGCTACTATTTAATAAAACAAAATCTGTAGAATTCACGTTTTGTAATGACACTG |
| | TCGTCATTCCATGCTTTGTTACTAATATGGAGGCACAAAACACTACTGAAGTAT |
| | ACGTAAAGTGGAAATTTAAAGGAAGAGATATTTACACCTTTGATGGAGCTCTAA |
| | ACAAGTCCACTGTCCCCACTGACTTTAGTAGTGCAAAAATTGAAGTCTCACAAT |
| | TACTAAAAGGAGATGCCTCTTTGAAGATGGATAAGAGTGATGCTGTCTCACAC |
| | ACAGGAAACTACACTTGTGAAGTAACAGAATTAACCAGAGAAGGTGAAACGAT |
| | CATCGAGCTAAAATATCGTGTTGTTTCATGGTTTTCTCCAAATGAAAAT |
| 62 | CAGCTACTATTTAATAAAACAAAATCTGTAGAATTCACGTTTGGTAATGACACT |
| | GTCGTCATTCCATGCTTTGTTACTAATATGGAGGCACAAAACACTACTGAAGTA |
| | TACGTAAAGTGGAAATTTAAAGGAAGAGATATTTACACCTTTGATGGAGCTCTA |
| | AACAAGTCCACTGTCCCCACTGACTTTAGTAGTGCAAAAATTGAAGTCTCACAA |
| | TTACTAAAAGGAGATGCCTCTTTGAAGATGGATAAGAGTGATGCTGTCTCACAC |
| | ACAGGAAACTACACTTGTGAAGTAACAGAATTAACCAGAGAAGGTGAAACGAT |
| | CATCGAGCTAAAATATCGTGTTGTTTCATGGTTTTCTCCAAATGAAAAT |
| 63 | CTCGAGCCGAAATCTTGTGACAAAACTCACACATGCCCACCGTGCCCAGCACC |
| | TGAAGCTGCAGGGGGACCGTCAGTCTTCCTCTTCCCCCCAAAACCCAAGGAC |
| | ACCCTCATGATCTCCCGGACCCCTGAGGTCACATGCGTGGTGGTGGACGTGA |
| | GCCACGAAGACCCTGAGGTCAAGTTCAACTGGTACGTGGACGGCGTGGAGGT |
| | GCATAATGCCAAGACAAAGCCGCGGGAGGAGCAGTACAACAGCACGTACCGG |
| | GTGGTCAGCGTCCTCACCGTCCTGCACCAGGACTGGCTGAATGGCAAGGAGT |
| | ACAAGTGCAAGGTCTCCAACAAAGCCCTCCCAGCCCCCATCGAGAAAACCATC |
| | TCCAAAGCCAAAGGGCAGCCCCGAGAACCACAGGTGTACACCCTGCCCCCAT |
| | CCCGGGAGGAGATGACCAAGAACCAGGTCAGCCTGACCTGCCTGGTCAAAGG |
| | CTTCTATCCCAGCGACATCGCCGTGGAGTGGGAGAGCAATGGGCAGCCGGAG |
| | AACAACTACAAGACCACGCCTCCCGTGCTGGACTCCGACGGCTCCTTCTTCCT |
| | CTACAGCAAGCTCACCGTGGACAAGAGCAGGTGGCAGCAGGGGAACGTCTTC |
| | TCATGCTCCGTGATGCATGAGGCTCTGCACAACCACTACACGCAGAAGAGCCT |
| | CTCCCTGTCTCCGGGTAAA |
| 64 | ATGTGGCCCCTGGTAGCGGCGCTGTTGCTGGGCTCGGCGTGCTGCGGATCA |
| | GCTCAGCTACTATTTAATAAAACAAAATCTGTAGAATTCACGTTTTGTAATGACA |
| | CTGTCGTCATTCCATGCTTTGTTACTAATATGGAGGCACAAAACACTACTGAAG |
| | TATACGTAAAGTGGAAATTTAAAGGAAGAGATATTTACACCTTTGATGGAGCTC |
| | TAAACAAGTCCACTGTCCCCACTGACTTTAGTAGTGCAAAAATTGAAGTCTCAC |
| | AATTACTAAAAGGAGATGCCTCTTTGAAGATGGATAAGAGTGATGCTGTCTCAC |
| | ACACAGGAAACTACACTTGTGAAGTAACAGAATTAACCAGAGAAGGTGAAACG |
| | ATCATCGAGCTAAAATATCGTGTTGTTTCATGGTTTTCTCCAAATGAAAATCTC |
| | GAGCCGAAATCTTGTGACAAAACTCACACATGCCCACCGTGCCCAGCACCTGA |
| | AGCTGCAGGGGGACCGTCAGTCTTCCTCTTCCCCCCAAAACCCAAGGACACC |
| | CTCATGATCTCCCGGACCCCTGAGGTCACATGCGTGGTGGTGGACGTGAGCC |
| | ACGAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGGGTG |
| | GTCAGCGTCCTCACCGTCCTGCACCAGGACTGGCTGAATGGCAAGGAGTACA |
| | AGTGCAAGGTCTCCAACAAAGCCCTCCCAGCCCCCATCGAGAAAACCATCTCC |
| | AAAGCCAAAGGGCAGCCCCGAGAACCACAGGTGTACACCCTGCCCCCATCCC |
| | GGGAGGAGATGACCAAGAACCAGGTCAGCCTGACCTGCCTGGTCAAAGGCTT |
| | CTATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCCTCT |
| | ACAGCAAGCTCACCGTGGACAAGAGCAGGTGGCAGCAGGGGAACGTCTTCTC |
| | ATGCTCCGTGATGCATGAGGCTCTGCACAACCACTACACGCAGAAGAGCCTCT |
| | CCCTGTCTCCGGGTAAATGA |
| 65 | GGCGGCGGCGGATCC |
| 66 | GGAGGTGGTGGATCTGGAGGTGGAGGTAGC |
| 67 | TCAGCTAGCACCAAGGGCCCCAGCGTGTTCCCCCTGGCCCCCAGCAGCAAGA |
| | GCACCAGCGGCGGCACAGCCGCCCTGGGCTGCCTGGTGAAGGACTACTTCC |
| | CCGAGCCCGTGACCGTGTCCTGGAACAGCGGAGCCCTGACCTCCGGCGTGC |
| | ACACCTTCCCCGCCGTGCTGCAGAGCAGCGGCCTGTACAGCCTGTCCAGCGT |
| | GGTGACAGTGCCCAGCAGCAGCCTGGGCACCCAGACCTACATCTGCAACGTG |
| | AACCACAAGCCCAGCAACACCAAGGTGGACAAGAGAGTG |
| 68 | GAGCCCAAGAGCTGCGACAAGACCCACACCTGCCCCCCCTGCCCAGCCCCA |
| | GAGGCAGCGGGCGGACCCTCCGTGTTCCTGTTCCCCCCCAAGCCCAAGGACA |
| | CCCTGATGATCAGCAGGACCCCCGAGGTGACCTGCGTGGTGGTGGACGTGA |
| | GCCACGAGGACCCAGAGGTGAAGTTCAACTGGTACGTGGACGGCGTGGAGG |
| | TGCACAACGCCAAGACCAAGCCCAGAGAGGAGCAGTACAACAGCACCTACAG |
| | GGTGGTGTCCGTGCTGACCGTGCTGCACCAGGACTGGCTGAACGGCAAGGAA |
| | TACAAGTGCAAGGTCTCCAACAAGGCCCTGCCAGCCCCCATCGAAAAGACCAT |
| | CAGCAAGGCCAAGGGCCAGCCACGGGAGCCCCAGGTGTACACCCTGCCCCC |
| | CTCCCGGGAGGAGATGACCAAGAACCAGGTGTCCCTGACCTGTCTGGTGAAG |
| | GGCTTCTACCCCAGCGACATCGCCGTGGAGTGGGAGAGCAACGGCCAGCCC |
| | GAGAACAACTACAAGACCACCCCCCCAGTGCTGGACAGCGACGGCAGCTTCT |
| | TCCTGTACAGCAAGCTGACCGTGGACAAGTCCAGGTGGCAGCAGGGCAACGT |
| | GTTCAGCTGCAGCGTGATGCACGAGGCCCTGCACAACCACTACACCCAGAAG |
| | AGCCTGAGCCTGTCCCCCGGCAAG |
| 69 | TCAGCTAGCACCAAGGGCCCCAGCGTGTTCCCCCTGGCCCCCAGCAGCAAGA |
| | GCACCAGCGGCGGCACAGCCGCCCTGGGCTGCCTGGTGAAGGACTACTTCC |
| | CCGAGCCCGTGACCGTGTCCTGGAACAGCGGAGCCCTGACCTCCGGCGTGC |
| | ACACCTTCCCCGCCGTGCTGCAGAGCAGCGGCCTGTACAGCCTGTCCAGCGT |
| | GGTGACAGTGCCCAGCAGCAGCCTGGGCACCCAGACCTACATCTGCAACGTG |
| | AACCACAAGCCCAGCAACACCAAGGTGGACAAGAGAGTGGAGCCCAAGAGCT |
| | GCGACAAGACCCACACCTGCCCCCCCTGCCCAGCCCCAGAGGCAGCGGGCG |
| | GACCCTCCGTGTTCCTGTTCCCCCCCAAGCCCAAGGACACCCTGATGATCAG |
| | CAGGACCCCCGAGGTGACCTGCGTGGTGGTGGACGTGAGCCACGAGGACCC |
| | AGAGGTGAAGTTCAACTGGTACGTGGACGGCGTGGAGGTGCACAACGCCAAG |
| | ACCAAGCCCAGAGAGGAGCAGTACAACAGCACCTACAGGGTGGTGTCCGTGC |
| | TGACCGTGCTGCACCAGGACTGGCTGAACGGCAAGGAATACAAGTGCAAGGT |
| | CTCCAACAAGGCCCTGCCAGCCCCCATCGAAAAGACCATCAGCAAGGCCAAG |
| | GGCCAGCCACGGGAGCCCCAGGTGTACACCCTGCCCCCCTCCCGGGAGGAG |
| | ATGACCAAGAACCAGGTGTCCCTGACCTGTCTGGTGAAGGGCTTCTACCCCA |
| | GCGACATCGCCGTGGAGTGGGAGAGCAACGGCCAGCCCGAGAACAACTACA |
| | AGACCACCCCCCCAGTGCTGGACAGCGACGGCAGCTTCTTCCTGTACAGCAA |
| | GCTGACCGTGGACAAGTCCAGGTGGCAGCAGGGCAACGTGTTCAGCTGCAGC |
| | GTGATGCACGAGGCCCTGCACAACCACTACACCCAGAAGAGCCTGAGCCTGT |
| | CCCCCGGCAAG |
| 70 | CGTACGGTGGCCGCTCCCAGCGTGTTCATCTTCCCCCCCAGCGACGAGCAGC |
| | TGAAGAGCGGCACCGCCAGCGTGGTGTGCCTGCTGAACAACTTCTACCCCCG |
| | GGAGGCCAAGGTGCAGTGGAAGGTGGACAACGCCCTGCAGAGCGGCAACAG |
| | CCAGGAGAGCGTCACCGAGCAGGACAGCAAGGACTCCACCTACAGCCTGAGC |
| | AGCACCCTGACCCTGAGCAAGGCCGACTACGAGAAGCATAAGGTGTACGCCT |
| | GCGAGGTGACCCACCAGGGCCTGTCCAGCCCCGTGACCAAGAGCTTCAACAG |
| | GGGCGAGTGC |
| 71 | ATGTGGCCCCTGGTAGCGGCGCTGTTGCTGGGCTCGGCGTGCTGCGGATCA |
| | GCTCAGCTACTATTTAATAAAACAAAATCTGTAGAATTCACGTTTGGTAATGAC |
| | ACTGTCGTCATTCCATGCTTTGTTACTAATATGGAGGCACAAAACACTACTGAA |
| | GTATACGTAAAGTGGAAATTTAAAGGAAGAGATATTTACACCTTTGATGGAGCT |
| | CTAAACAAGTCCACTGTCCCCACTGACTTTAGTAGTGCAAAAATTGAAGTCTCA |
| | CAATTACTAAAAGGAGATGCCTCTTTGAAGATGGATAAGAGTGATGCTGTCTCA |
| | CACACAGGAAACTACACTTGTGAAGTAACAGAATTAACCAGAGAAGGTGAAAC |
| | GATCATCGAGCTAAAATATCGTGTTGTTTCATGGTTTTCTCCAAATGAAAATGG |
| | AGGTGGTGGATCTGGAGGTGGAGGTAGCTCAGCTAGCACCAAGGGCCCCAG |
| | CGTGTTCCCCCTGGCCCCCAGCAGCAAGAGCACCAGCGGCGGCACAGCCGC |
| | CCTGGGCTGCCTGGTGAAGGACTACTTCCCCGAGCCCGTGACCGTGTCCTGG |
| | AACAGCGGAGCCCTGACCTCCGGCGTGCACACCTTCCCCGCCGTGCTGCAGA |
| | GCAGCGGCCTGTACAGCCTGTCCAGCGTGGTGACAGTGCCCAGCAGCAGCCT |
| | GGGCACCCAGACCTACATCTGCAACGTGAACCACAAGCCCAGCAACACCAAG |
| | GTGGACAAGAGAGTGGAGCCCAAGAGCTGCGACAAGACCCACACCTGCCCCC |
| | CCTGCCCAGCCCCAGAGGCAGCGGGCGGACCCTCCGTGTTCCTGTTCCCCC |
| | CCAAGCCCAAGGACACCCTGATGATCAGCAGGACCCCCGAGGTGACCTGCGT |
| | GGTGGTGGACGTGAGCCACGAGGACCCAGAGGTGAAGTTCAACTGGTACGTG |
| | GACGGCGTGGAGGTGCACAACGCCAAGACCAAGCCCAGAGAGGAGCAGTAC |
| | AACAGCACCTACAGGGTGGTGTCCGTGCTGACCGTGCTGCACCAGGACTGGC |
| | TGAACGGCAAGGAATACAAGTGCAAGGTCTCCAACAAGGCCCTGCCAGCCCC |
| | CATCGAAAAGACCATCAGCAAGGCCAAGGGCCAGCCACGGGAGCCCCAGGT |
| | GTACACCCTGCCCCCCTCCCGGGAGGAGATGACCAAGAACCAGGTGTCCCTG |
| | ACCTGTCTGGTGAAGGGCTTCTACCCCAGCGACATCGCCGTGGAGTGGGAGA |
| | GCAACGGCCAGCCCGAGAACAACTACAAGACCACCCCCCCAGTGCTGGACAG |
| | CGACGGCAGCTTCTTCCTGTACAGCAAGCTGACCGTGGACAAGTCCAGGTGG |
| | CAGCAGGGCAACGTGTTCAGCTGCAGCGTGATGCACGAGGCCCTGCACAACC |
| | ACTACACCCAGAAGAGCCTGAGCCTGTCCCCCGGCAAGTGA |
| 72 | ATGTGGCCCCTGGTAGCGGCGCTGTTGCTGGGCTCGGCGTGCTGCGGATCA |
| | GCTCAGCTACTATTTAATAAAACAAAATCTGTAGAATTCACGTTTGGTAATGAC |
| | ACTGTCGTCATTCCATGCTTTGTTACTAATATGGAGGCACAAAACACTACTGAA |
| | GTATACGTAAAGTGGAAATTTAAAGGAAGAGATATTTACACCTTTGATGGAGCT |
| | CTAAACAAGTCCACTGTCCCCACTGACTTTAGTAGTGCAAAAATTGAAGTCTCA |
| | CAATTACTAAAAGGAGATGCCTCTTTGAAGATGGATAAGAGTGATGCTGTCTCA |
| | CACACAGGAAACTACACTTGTGAAGTAACAGAATTAACCAGAGAAGGTGAAAC |
| | GATCATCGAGCTAAAATATCGTGTTGTTTCATGGTTTTCTCCAAATGAAAATGG |
| | AGGTGGTGGATCTGGAGGTGGAGGTAGCCGTACGGTGGCCGCTCCCAGCGT |
| | GTTCATCTTCCCCCCCAGCGACGAGCAGCTGAAGAGCGGCACCGCCAGCGTG |
| | GTGTGCCTGCTGAACAACTTCTACCCCCGGGAGGCCAAGGTGCAGTGGAAGG |
| | TGGACAACGCCCTGCAGAGCGGCAACAGCCAGGAGAGCGTCACCGAGCAGG |
| | ACAGCAAGGACTCCACCTACAGCCTGAGCAGCACCCTGACCCTGAGCAAGGC |
| | CGACTACGAGAAGCATAAGGTGTACGCCTGCGAGGTGACCCACCAGGGCCTG |
| | TCCAGCCCCGTGACCAAGAGCTTCAACAGGGGCGAGTGCTGA |
| 73 | ATGTGGCCCCTGGTAGCGGCGCTGTTGCTGGGCTCGGCGTGCTGCGGATCA |
| | GCTCAGCTACTATTTAATAAAACAAAATCTGTAGAATTCACGTTTTGTAATGACA |
| | CTGTCGTCATTCCATGCTTTGTTACTAATATGGAGGCACAAAACACTACTGAAG |
| | TATACGTAAAGTGGAAATTTAAAGGAAGAGATATTTACACCTTTGATGGAGCTC |
| | TAAACAAGTCCACTGTCCCCACTGACTTTAGTAGTGCAAAAATTGAAGTCTCAC |
| | AATTACTAAAAGGAGATGCCTCTTTGAAGATGGATAAGAGTGATGCTGTCTCAC |
| | ACACAGGAAACTACACTTGTGAAGTAACAGAATTAACCAGAGAAGGTGAAACG |
| | ATCATCGAGCTAAAATATCGTGTTGTTTCATGGTTTTCTCCAAATGAAAATGGA |
| | GGTGGTGGATCTGGAGGTGGAGGTAGCTCAGCTAGCACCAAGGGCCCCAGC |
| | GTGTTCCCCCTGGCCCCCAGCAGCAAGAGCACCAGCGGCGGCACAGCCGCC |
| | CTGGGCTGCCTGGTGAAGGACTACTTCCCCGAGCCCGTGACCGTGTCCTGGA |
| | ACAGCGGAGCCCTGACCTCCGGCGTGCACACCTTCCCCGCCGTGCTGCAGA |
| | GCAGCGGCCTGTACAGCCTGTCCAGCGTGGTGACAGTGCCCAGCAGCAGCCT |
| | GGGCACCCAGACCTACATCTGCAACGTGAACCACAAGCCCAGCAACACCAAG |
| | GTGGACAAGAGAGTGGAGCCCAAGAGCTGCGACAAGACCCACACCTGCCCCC |
| | CCTGCCCAGCCCCAGAGGCAGCGGGCGGACCCTCCGTGTTCCTGTTCCCCC |
| | CCAAGCCCAAGGACACCCTGATGATCAGCAGGACCCCCGAGGTGACCTGCGT |
| | GGTGGTGGACGTGAGCCACGAGGACCCAGAGGTGAAGTTCAACTGGTACGTG |
| | GACGGCGTGGAGGTGCACAACGCCAAGACCAAGCCCAGAGAGGAGCAGTAC |
| | AACAGCACCTACAGGGTGGTGTCCGTGCTGACCGTGCTGCACCAGGACTGGC |
| | TGAACGGCAAGGAATACAAGTGCAAGGTCTCCAACAAGGCCCTGCCAGCCCC |
| | CATCGAAAAGACCATCAGCAAGGCCAAGGGCCAGCCACGGGAGCCCCAGGT |
| | GTACACCCTGCCCCCCTCCCGGGAGGAGATGACCAAGAACCAGGTGTCCCTG |
| | ACCTGTCTGGTGAAGGGCTTCTACCCCAGCGACATCGCCGTGGAGTGGGAGA |
| | GCAACGGCCAGCCCGAGAACAACTACAAGACCACCCCCCCAGTGCTGGACAG |
| | CGACGGCAGCTTCTTCCTGTACAGCAAGCTGACCGTGGACAAGTCCAGGTGG |
| | CAGCAGGGCAACGTGTTCAGCTGCAGCGTGATGCACGAGGCCCTGCACAACC |
| | ACTACACCCAGAAGAGCCTGAGCCTGTCCCCCGGCAAGTGA |
| 74 | ATGTGGCCCCTGGTAGCGGCGCTGTTGCTGGGCTCGGCGTGCTGCGGATCA |
| | GCTCAGCTACTATTTAATAAAACAAAATCTGTAGAATTCACGTTTTGTAATGACA |
| | CTGTCGTCATTCCATGCTTTGTTACTAATATGGAGGCACAAAACACTACTGAAG |
| | TATACGTAAAGTGGAAATTTAAAGGAAGAGATATTTACACCTTTGATGGAGCTC |
| | TAAACAAGTCCACTGTCCCCACTGACTTTAGTAGTGCAAAAATTGAAGTCTCAC |
| | AATTACTAAAAGGAGATGCCTCTTTGAAGATGGATAAGAGTGATGCTGTCTCAC |
| | ACACAGGAAACTACACTTGTGAAGTAACAGAATTAACCAGAGAAGGTGAAACG |
| | ATCATCGAGCTAAAATATCGTGTTGTTTCATGGTTTTCTCCAAATGAAAATGGA |
| | GGTGGTGGATCTGGAGGTGGAGGTAGCCGTACGGTGGCCGCTCCCAGCGTG |
| | TTCATCTTCCCCCCCAGCGACGAGCAGCTGAAGAGCGGCACCGCCAGCGTGG |
| | TGTGCCTGCTGAACAACTTCTACCCCCGGGAGGCCAAGGTGCAGTGGAAGGT |
| | GGACAACGCCCTGCAGAGCGGCAACAGCCAGGAGAGCGTCACCGAGCAGGA |
| | CAGCAAGGACTCCACCTACAGCCTGAGCAGCACCCTGACCCTGAGCAAGGCC |
| | GACTACGAGAAGCATAAGGTGTACGCCTGCGAGGTGACCCACCAGGGCCTGT |
| | CCAGCCCCGTGACCAAGAGCTTCAACAGGGGCGAGTGCTGA |
| 75 | ATGTGGCCCCTGGTAGCGGCGCTGTTGCTGGGCTCGGCGTGCTGCGGATCA |
| | GCTCAGCTACTATTTAATAAAACAAAATCTGTAGAATTCACGTTTTGTAATGACA |
| | CTGTCGTCATTCCATGCTTTGTTACTAATATGGAGGCACAAAACACTACTGAAG |
| | TATACGTAAAGTGGAAATTTAAAGGAAGAGATATTTACACCTTTGATGGAGCTC |
| | TAAACAAGTCCACTGTCCCCACTGACTTTAGTAGTGCAAAAATTGAAGTCTCAC |
| | AATTACTAAAAGGAGATGCCTCTTTGAAGATGGATAAGAGTGATGCTGTCTCAC |
| | ACACAGGAAACTACACTTGTGAAGTAACAGAATTAACCAGAGAAGGTGAAACG |
| | ATCATCGAGCTAAAATATCGTGTTGTTTCAGGCGGCGGCGGATCCAGCGCTAG |
| | CACCAAGGGCCCCAGCGTGTTCCCCCTGGCCCCCAGCAGCAAGAGCACCAG |
| | CGGCGGCACAGCCGCCCTGGGCTGCCTGGTGAAGGACTACTTCCCCGAGCC |
| | CGTGACCGTGTCCTGGAACAGCGGAGCCCTGACCTCCGGCGTGCACACCTTC |
| | CCCGCCGTGCTGCAGAGCAGCGGCCTGTACAGCCTGTCCAGCGTGGTGACA |
| | GTGCCCAGCAGCAGCCTGGGCACCCAGACCTACATCTGCAACGTGAACCACA |
| | AGCCCAGCAACACCAAGGTGGACAAGAGAGTGGAGCCCAAGAGCTGCGGCG |
| | GCGGCGGCTCCGGCGGCGGCGGATCCAGCGCTAGCACCAAGGGCCCCAGC |
| | GTGTTCCCCCTGGCCCCCAGCAGCAAGAGCACCAGCGGCGGCACAGCCGCC |
| | CTGGGCTGCCTGGTGAAGGACTACTTCCCCGAGCCCGTGACCGTGTCCTGGA |
| | ACAGCGGAGCCCTGACCTCCGGCGTGCACACCTTCCCCGCCGTGCTGCAGA |
| | GCAGCGGCCTGTACAGCCTGTCCAGCGTGGTGACAGTGCCCAGCAGCAGCCT |
| | GGGCACCCAGACCTACATCTGCAACGTGAACCACAAGCCCAGCAACACCAAG |
| | GTGGACAAGAGAGTGGAGCCCAAGAGCTGCGACAAGACCCACACCTGCCCCC |
| | CCTGCCCAGCCCCAGAGGCAGCGGGCGGACCCTCCGTGTTCCTGTTCCCCC |
| | CCAAGCCCAAGGACACCCTGATGATCAGCAGGACCCCCGAGGTGACCTGCGT |
| | GGTGGTGGACGTGAGCCACGAGGACCCAGAGGTGAAGTTCAACTGGTACGTG |
| | GACGGCGTGGAGGTGCACAACGCCAAGACCAAGCCCAGAGAGGAGCAGTAC |
| | AACAGCACCTACAGGGTGGTGTCCGTGCTGACCGTGCTGCACCAGGACTGGC |
| | TGAACGGCAAGGAATACAAGTGCAAGGTCTCCAACAAGGCCCTGCCAGCCCC |
| | CATCGAAAAGACCATCAGCAAGGCCAAGGGCCAGCCACGGGAGCCCCAGGT |
| | GTACACCCTGCCCCCCTCCCGGGAGGAGATGACCAAGAACCAGGTGTCCCTG |
| | ACCTGTCTGGTGAAGGGCTTCTACCCCAGCGACATCGCCGTGGAGTGGGAGA |
| | GCAACGGCCAGCCCGAGAACAACTACAAGACCACCCCCCCAGTGCTGGACAG |
| | CGACGGCAGCTTCTTCCTGTACAGCAAGCTGACCGTGGACAAGTCCAGGTGG |
| | CAGCAGGGCAACGTGTTCAGCTGCAGCGTGATGCACGAGGCCCTGCACAACC |
| | ACTACACCCAGAAGAGCCTGAGCCTGTCCCCCGGCAAGTGA |
| 76 | ATGTGGCCCCTGGTAGCGGCGCTGTTGCTGGGCTCGGCGTGCTGCGGATCA |
| | GCTCAGCTACTATTTAATAAAACAAAATCTGTAGAATTCACGTTTTGTAATGACA |
| | CTGTCGTCATTCCATGCTTTGTTACTAATATGGAGGCACAAAACACTACTGAAG |
| | TATACGTAAAGTGGAAATTTAAAGGAAGAGATATTTACACCTTTGATGGAGCTC |
| | TAAACAAGTCCACTGTCCCCACTGACTTTAGTAGTGCAAAAATTGAAGTCTCAC |
| | AATTACTAAAAGGAGATGCCTCTTTGAAGATGGATAAGAGTGATGCTGTCTCAC |
| | ACACAGGAAACTACACTTGTGAAGTAACAGAATTAACCAGAGAAGGTGAAACG |
| | ATCATCGAGCTAAAATATCGTGTTGTTTCAGGCGGCGGCGGATCCCGTACGGT |
| | GGCCGCTCCCAGCGTGTTCATCTTCCCCCCCAGCGACGAGCAGCTGAAGAGC |
| | GGCACCGCCAGCGTGGTGTGCCTGCTGAACAACTTCTACCCCCGGGAGGCCA |
| | AGGTGCAGTGGAAGGTGGACAACGCCCTGCAGAGCGGCAACAGCCAGGAGA |
| | GCGTCACCGAGCAGGACAGCAAGGACTCCACCTACAGCCTGAGCAGCACCCT |
| | GACCCTGAGCAAGGCCGACTACGAGAAGCATAAGGTGTACGCCTGCGAGGTG |
| | ACCCACCAGGGCCTGTCCAGCCCCGTGACCAAGAGCTTCAACAGGGGCGAGT |
| | GCGGCGGCGGCGGCTCCGGCGGCGGCGGATCCCGTACGGTGGCCGCTCCC |
| | AGCGTGTTCATCTTCCCCCCCAGCGACGAGCAGCTGAAGAGCGGCACCGCCA |
| | GCGTGGTGTGCCTGCTGAACAACTTCTACCCCCGGGAGGCCAAGGTGCAGTG |
| | GAAGGTGGACAACGCCCTGCAGAGCGGCAACAGCCAGGAGAGCGTCACCGA |
| | GCAGGACAGCAAGGACTCCACCTACAGCCTGAGCAGCACCCTGACCCTGAGC |
| | AAGGCCGACTACGAGAAGCATAAGGTGTACGCCTGCGAGGTGACCCACCAGG |
| | GCCTGTCCAGCCCCGTGACCAAGAGCTTCAACAGGGGCGAGTGCTGA |
| 77 | ATGTGGCCCCTGGTAGCGGCGCTGTTGCTGGGCTCGGCGTGCTGCGGATCA |
| | GCTCAGCTACTATTTAATAAAACAAAATCTGTAGAATTCACGTTTTGTAATGACA |
| | CTGTCGTCATTCCATGCTTTGTTACTAATATGGAGGCACAAAACACTACTGAAG |
| | TATACGTAAAGTGGAAATTTAAAGGAAGAGATATTTACACCTTTGATGGAGCTC |
| | TAAACAAGTCCACTGTCCCCACTGACTTTAGTAGTGCAAAAATTGAAGTCTCAC |
| | AATTACTAAAAGGAGATGCCTCTTTGAAGATGGATAAGAGTGATGCTGTCTCAC |
| | ACACAGGAAACTACACTTGTGAAGTAACAGAATTAACCAGAGAAGGTGAAACG |
| | ATCATCGAGCTAAAATATCGTGTTGTTTCATGGTTTTCTCCAAATGAAAATGAG |
| | GTGCAATTGGTGGAAAGCGGCGGAGGACTGGTGCAGCCCGGCAGAAGCCTG |
| | AGACTGAGCTGCGCCGCCAGCGGCTTCACCTTCGACGACTACGCCATGCACT |
| | GGGTCCGCCAGGCCCCTGGCAAGGGACTGGAATGGGTGTCCGCCATCACCT |
| | GGAACAGCGGCCACATCGACTACGCCGACAGCGTGGAAGGCCGGTTCACCAT |
| | CAGCCGGGACAACGCCAAGAACAGCCTGTACCTGCAGATGAACAGCCTGCGG |
| | GCCGAGGACACCGCCGTGTACTACTGCGCCAAGGTGTCCTACCTGAGCACCG |
| | CCAGCAGCCTGGACTACTGGGGCCAGGGCACACTGGTCACAGTCAGCTCAGC |
| | TAGCACCAAGGGCCCCAGCGTGTTCCCCCTGGCCCCCAGCAGCAAGAGCACC |
| | AGCGGCGGCACAGCCGCCCTGGGCTGCCTGGTGAAGGACTACTTCCCCGAG |
| | CCCGTGACCGTGTCCTGGAACAGCGGAGCCCTGACCTCCGGCGTGCACACCT |
| | TCCCCGCCGTGCTGCAGAGCAGCGGCCTGTACAGCCTGTCCAGCGTGGTGAC |
| | AGTGCCCAGCAGCAGCCTGGGCACCCAGACCTACATCTGCAACGTGAACCAC |
| | AAGCCCAGCAACACCAAGGTGGACAAGAGAGTGGAGCCCAAGAGCTGCGACA |
| | AGACCCACACCTGCCCCCCCTGCCCAGCCCCAGAGCTGCTGGGCGGACCCT |
| | CCGTGTTCCTGTTCCCCCCCAAGCCCAAGGACACCCTGATGATCAGCAGGAC |
| | CCCCGAGGTGACCTGCGTGGTGGTGGACGTGAGCCACGAGGACCCAGAGGT |
| | GAAGTTCAACTGGTACGTGGACGGCGTGGAGGTGCACAACGCCAAGACCAAG |
| | CCCAGAGAGGAGCAGTACAACAGCACCTACAGGGTGGTGTCCGTGCTGACCG |
| | TGCTGCACCAGGACTGGCTGAACGGCAAGGAATACAAGTGCAAGGTCTCCAA |
| | CAAGGCCCTGCCAGCCCCCATCGAAAAGACCATCAGCAAGGCCAAGGGCCAG |
| | CCACGGGAGCCCCAGGTGTACACCCTGCCCCCCTCCCGGGAGGAGATGACC |
| | AAGAACCAGGTGTCCCTGACCTGTCTGGTGAAGGGCTTCTACCCCAGCGACA |
| | TCGCCGTGGAGTGGGAGAGCAACGGCCAGCCCGAGAACAACTACAAGACCAC |
| | CCCCCCAGTGCTGGACAGCGACGGCAGCTTCTTCCTGTACAGCAAGCTGACC |
| | GTGGACAAGTCCAGGTGGCAGCAGGGCAACGTGTTCAGCTGCAGCGTGATGC |
| | ACGAGGCCCTGCACAACCACTACACCCAGAAGAGCCTGAGCCTGTCCCCCGG |
| | CAAGTGA |
| 78 | ATGTGGCCCCTGGTAGCGGCGCTGTTGCTGGGCTCGGCGTGCTGCGGATCA |
| | GCTCAGCTACTATTTAATAAAACAAAATCTGTAGAATTCACGTTTTGTAATGACA |
| | CTGTCGTCATTCCATGCTTTGTTACTAATATGGAGGCACAAAACACTACTGAAG |
| | TATACGTAAAGTGGAAATTTAAAGGAAGAGATATTTACACCTTTGATGGAGCTC |
| | TAAACAAGTCCACTGTCCCCACTGACTTTAGTAGTGCAAAAATTGAAGTCTCAC |
| | AATTACTAAAAGGAGATGCCTCTTTGAAGATGGATAAGAGTGATGCTGTCTCAC |
| | ACACAGGAAACTACACTTGTGAAGTAACAGAATTAACCAGAGAAGGTGAAACG |
| | ATCATCGAGCTAAAATATCGTGTTGTTTCATGGTTTTCTCCAAATGAAAATGATA |
| | TCCAGATGACCCAGAGCCCCAGCAGCCTGAGCGCCAGCGTGGGCGACAGAG |
| | TGACCATCACCTGTCGGGCCAGCCAGGGCATCCGGAACTACCTGGCCTGGTA |
| | TCAGCAGAAGCCCGGCAAGGCCCCCAAGCTGCTGATCTACGCCGCCAGCACC |
| | CTGCAGAGCGGCGTGCCAAGCAGATTCAGCGGCAGCGGCTCCGGCACCGAC |
| | TTCACCCTGACCATCAGCAGCCTGCAGCCCGAGGACGTGGCCACCTACTACT |
| | GCCAGCGGTACAACAGAGCCCCCTACACCTTCGGCCAGGGCACCAAGGTGGA |
| | AATCAAGCGTACGGTGGCCGCTCCCAGCGTGTTCATCTTCCCCCCCAGCGAC |
| | GAGCAGCTGAAGAGCGGCACCGCCAGCGTGGTGTGCCTGCTGAACAACTTCT |
| | ACCCCCGGGAGGCCAAGGTGCAGTGGAAGGTGGACAACGCCCTGCAGAGCG |
| | GCAACAGCCAGGAGAGCGTCACCGAGCAGGACAGCAAGGACTCCACCTACAG |
| | CCTGAGCAGCACCCTGACCCTGAGCAAGGCCGACTACGAGAAGCATAAGGTG |
| | TACGCCTGCGAGGTGACCCACCAGGGCCTGTCCAGCCCCGTGACCAAGAGCT |
| | TCAACAGGGGCGAGTGCTGA |
| 79 | ATGTGGCCCCTGGTAGCGGCGCTGTTGCTGGGCTCGGCGTGCTGCGGATCA |
| | GCTCAGCTACTATTTAATAAAACAAAATCTGTAGAATTCACGTTTTGTAATGACA |
| | CTGTCGTCATTCCATGCTTTGTTACTAATATGGAGGCACAAAACACTACTGAAG |
| | TATACGTAAAGTGGAAATTTAAAGGAAGAGATATTTACACCTTTGATGGAGCTC |
| | TAAACAAGTCCACTGTCCCCACTGACTTTAGTAGTGCAAAAATTGAAGTCTCAC |
| | AATTACTAAAAGGAGATGCCTCTTTGAAGATGGATAAGAGTGATGCTGTCTCAC |
| | ACACAGGAAACTACACTTGTGAAGTAACAGAATTAACCAGAGAAGGTGAAACG |
| | ATCATCGAGCTAAAATATCGTGTTGTTTCATGGTTTTCTCCAAATGAAAATGGA |
| | GGTGGTGGATCTGGAGGTGGAGGATCCGAGGTCCAATTGGTGGAAAGCGGC |
| | GGAGGACTGGTGCAGCCCGGCAGAAGCCTGAGACTGAGCTGCGCCGCCAGC |
| | GGCTTCACCTTCGACGACTACGCCATGCACTGGGTCCGCCAGGCCCCTGGCA |
| | AGGGACTGGAATGGGTGTCCGCCATCACCTGGAACAGCGGCCACATCGACTA |
| | CGCCGACAGCGTGGAAGGCCGGTTCACCATCAGCCGGGACAACGCCAAGAA |
| | CAGCCTGTACCTGCAGATGAACAGCCTGCGGGCCGAGGACACCGCCGTGTAC |
| | TACTGCGCCAAGGTGTCCTACCTGAGCACCGCCAGCAGCCTGGACTACTGGG |
| | GCCAGGGCACACTGGTCACAGTCAGCTCAGCTAGCACCAAGGGCCCCAGCGT |
| | GTTCCCCCTGGCCCCCAGCAGCAAGAGCACCAGCGGCGGCACAGCCGCCCT |
| | GGGCTGCCTGGTGAAGGACTACTTCCCCGAGCCCGTGACCGTGTCCTGGAAC |
| | AGCGGAGCCCTGACCTCCGGCGTGCACACCTTCCCCGCCGTGCTGCAGAGC |
| | AGCGGCCTGTACAGCCTGTCCAGCGTGGTGACAGTGCCCAGCAGCAGCCTG |
| | GGCACCCAGACCTACATCTGCAACGTGAACCACAAGCCCAGCAACACCAAGG |
| | TGGACAAGAGAGTGGAGCCCAAGAGCTGCGACAAGACCCACACCTGCCCCCC |
| | CTGCCCAGCCCCAGAGCTGCTGGGCGGACCCTCCGTGTTCCTGTTCCCCCCC |
| | AAGCCCAAGGACACCCTGATGATCAGCAGGACCCCCGAGGTGACCTGCGTGG |
| | TGGTGGACGTGAGCCACGAGGACCCAGAGGTGAAGTTCAACTGGTACGTGGA |
| | CGGCGTGGAGGTGCACAACGCCAAGACCAAGCCCAGAGAGGAGCAGTACAA |
| | CAGCACCTACAGGGTGGTGTCCGTGCTGACCGTGCTGCACCAGGACTGGCTG |
| | AACGGCAAGGAATACAAGTGCAAGGTCTCCAACAAGGCCCTGCCAGCCCCCA |
| | TCGAAAAGACCATCAGCAAGGCCAAGGGCCAGCCACGGGAGCCCCAGGTGTA |
| | CACCCTGCCCCCCTCCCGGGAGGAGATGACCAAGAACCAGGTGTCCCTGACC |
| | TGTCTGGTGAAGGGCTTCTACCCCAGCGACATCGCCGTGGAGTGGGAGAGCA |
| | ACGGCCAGCCCGAGAACAACTACAAGACCACCCCCCCAGTGCTGGACAGCGA |
| | CGGCAGCTTCTTCCTGTACAGCAAGCTGACCGTGGACAAGTCCAGGTGGCAG |
| | CAGGGCAACGTGTTCAGCTGCAGCGTGATGCACGAGGCCCTGCACAACCACT |
| | ACACCCAGAAGAGCCTGAGCCTGTCCCCCGGCAAGTGA |
| 80 | ATGTGGCCCCTGGTAGCGGCGCTGTTGCTGGGCTCGGCGTGCTGCGGATCA |
| | GCTCAGCTACTATTTAATAAAACAAAATCTGTAGAATTCACGTTTTGTAATGACA |
| | CTGTCGTCATTCCATGCTTTGTTACTAATATGGAGGCACAAAACACTACTGAAG |
| | TATACGTAAAGTGGAAATTTAAAGGAAGAGATATTTACACCTTTGATGGAGCTC |
| | TAAACAAGTCCACTGTCCCCACTGACTTTAGTAGTGCAAAAATTGAAGTCTCAC |
| | AATTACTAAAAGGAGATGCCTCTTTGAAGATGGATAAGAGTGATGCTGTCTCAC |
| | ACACAGGAAACTACACTTGTGAAGTAACAGAATTAACCAGAGAAGGTGAAACG |
| | ATCATCGAGCTAAAATATCGTGTTGTTTCATGGTTTTCTCCAAATGAAAATGGA |
| | GGTGGTGGATCTGGAGGTGGAGGATCCGATATCCAGATGACCCAGAGCCCCA |
| | GCAGCCTGAGCGCCAGCGTGGGCGACAGAGTGACCATCACCTGTCGGGCCA |
| | GCCAGGGCATCCGGAACTACCTGGCCTGGTATCAGCAGAAGCCCGGCAAGG |
| | CCCCCAAGCTGCTGATCTACGCCGCCAGCACCCTGCAGAGCGGCGTGCCAAG |
| | CAGATTCAGCGGCAGCGGCTCCGGCACCGACTTCACCCTGACCATCAGCAGC |
| | CTGCAGCCCGAGGACGTGGCCACCTACTACTGCCAGCGGTACAACAGAGCCC |
| | CCTACACCTTCGGCCAGGGCACCAAGGTGGAAATCAAGCGTACGGTGGCCGC |
| | TCCCAGCGTGTTCATCTTCCCCCCCAGCGACGAGCAGCTGAAGAGCGGCACC |
| | GCCAGCGTGGTGTGCCTGCTGAACAACTTCTACCCCCGGGAGGCCAAGGTGC |
| | AGTGGAAGGTGGACAACGCCCTGCAGAGCGGCAACAGCCAGGAGAGCGTCA |
| | CCGAGCAGGACAGCAAGGACTCCACCTACAGCCTGAGCAGCACCCTGACCCT |
| | GAGCAAGGCCGACTACGAGAAGCATAAGGTGTACGCCTGCGAGGTGACCCAC |
| | CAGGGCCTGTCCAGCCCCGTGACCAAGAGCTTCAACAGGGGCGAGTGCTGA |
| 81 | GAGGTCCAATTGGTGGAAAGCGGCGGAGGACTGGTGCAGCCCGGCAGAAGC |
| | CTGAGACTGAGCTGCGCCGCCAGCGGCTTCACCTTCGACGACTACGCCATGC |
| | ACTGGGTCCGCCAGGCCCCTGGCAAGGGACTGGAATGGGTGTCCGCCATCA |
| | CCTGGAACAGCGGCCACATCGACTACGCCGACAGCGTGGAAGGCCGGTTCAC |
| | CATCAGCCGGGACAACGCCAAGAACAGCCTGTACCTGCAGATGAACAGCCTG |
| | CGGGCCGAGGACACCGCCGTGTACTACTGCGCCAAGGTGTCCTACCTGAGCA |
| | CCGCCAGCAGCCTGGACTACTGGGGCCAGGGCACACTGGTCACAGTCAGCTC |
| | AGCTAGCACCAAGGGCCCCAGCGTGTTCCCCCTGGCCCCCAGCAGCAAGAG |
| | CACCAGCGGCGGCACAGCCGCCCTGGGCTGCCTGGTGAAGGACTACTTCCC |
| | CGAGCCCGTGACCGTGTCCTGGAACAGCGGAGCCCTGACCTCCGGCGTGCA |
| | CACCTTCCCCGCCGTGCTGCAGAGCAGCGGCCTGTACAGCCTGTCCAGCGTG |
| | GTGACAGTGCCCAGCAGCAGCCTGGGCACCCAGACCTACATCTGCAACGTGA |
| | ACCACAAGCCCAGCAACACCAAGGTGGACAAGAGAGTGGAGCCCAAGAGCTG |
| | CGACAAGACCCACACCTGCCCCCCCTGCCCAGCCCCAGAGCTGCTGGGCGG |
| | ACCCTCCGTGTTCCTGTTCCCCCCCAAGCCCAAGGACACCCTGATGATCAGCA |
| | GGACCCCCGAGGTGACCTGCGTGGTGGTGGACGTGAGCCACGAGGACCCAG |
| | AGGTGAAGTTCAACTGGTACGTGGACGGCGTGGAGGTGCACAACGCCAAGAC |
| | CAAGCCCAGAGAGGAGCAGTACAACAGCACCTACAGGGTGGTGTCCGTGCTG |
| | ACCGTGCTGCACCAGGACTGGCTGAACGGCAAGGAATACAAGTGCAAGGTCT |
| | CCAACAAGGCCCTGCCAGCCCCCATCGAAAAGACCATCAGCAAGGCCAAGGG |
| | CCAGCCACGGGAGCCCCAGGTGTACACCCTGCCCCCCTCCCGGGAGGAGAT |
| | GACCAAGAACCAGGTGTCCCTGACCTGTCTGGTGAAGGGCTTCTACCCCAGC |
| | GACATCGCCGTGGAGTGGGAGAGCAACGGCCAGCCCGAGAACAACTACAAGA |
| | CCACCCCCCCAGTGCTGGACAGCGACGGCAGCTTCTTCCTGTACAGCAAGCT |
| | GACCGTGGACAAGTCCAGGTGGCAGCAGGGCAACGTGTTCAGCTGCAGCGT |
| | GATGCACGAGGCCCTGCACAACCACTACACCCAGAAGAGCCTGAGCCTGTCC |
| | CCCGGCAAG |
| 82 | GATATCCAGATGACCCAGAGCCCCAGCAGCCTGAGCGCCAGCGTGGGCGAC |
| | AGAGTGACCATCACCTGTCGGGCCAGCCAGGGCATCCGGAACTACCTGGCCT |
| | GGTATCAGCAGAAGCCCGGCAAGGCCCCCAAGCTGCTGATCTACGCCGCCAG |
| | CACCCTGCAGAGCGGCGTGCCAAGCAGATTCAGCGGCAGCGGCTCCGGCAC |
| | CGACTTCACCCTGACCATCAGCAGCCTGCAGCCCGAGGACGTGGCCACCTAC |
| | TACTGCCAGCGGTACAACAGAGCCCCCTACACCTTCGGCCAGGGCACCAAGG |
| | TGGAAATCAAGCGTACGGTGGCCGCTCCCAGCGTGTTCATCTTCCCCCCCAG |
| | CGACGAGCAGCTGAAGAGCGGCACCGCCAGCGTGGTGTGCCTGCTGAACAA |
| | CTTCTACCCCCGGGAGGCCAAGGTGCAGTGGAAGGTGGACAACGCCCTGCA |
| | GAGCGGCAACAGCCAGGAGAGCGTCACCGAGCAGGACAGCAAGGACTCCAC |
| | CTACAGCCTGAGCAGCACCCTGACCCTGAGCAAGGCCGACTACGAGAAGCAT |
| | AAGGTGTACGCCTGCGAGGTGACCCACCAGGGCCTGTCCAGCCCCGTGACCA |
| | AGAGCTTCAACAGGGGCGAGTGC |
| 83 | GAGGTCCAATTGGTGGAAAGCGGCGGAGGACTGGTGCAGCCCGGCAGAAGC |
| | CTGAGACTGAGCTGCGCCGCCAGCGGCTTCACCTTCGACGACTACGCCATGC |
| | ACTGGGTCCGCCAGGCCCCTGGCAAGGGACTGGAATGGGTGTCCGCCATCA |
| | CCTGGAACAGCGGCCACATCGACTACGCCGACAGCGTGGAAGGCCGGTTCAC |
| | CATCAGCCGGGACAACGCCAAGAACAGCCTGTACCTGCAGATGAACAGCCTG |
| | CGGGCCGAGGACACCGCCGTGTACTACTGCGCCAAGGTGTCCTACCTGAGCA |
| | CCGCCAGCAGCCTGGACTACTGGGGCCAGGGCACACTGGTCACAGTCAGC |
| 84 | GACTACGCCATGCAC |
| 85 | GCCATCACCTGGAACAGCGGCCACATCGACTACGCCGACAGCGTGGAAGGC |
| 86 | GTGTCCTACCTGAGCACCGCCAGCAGCCTGGACTAC |
| 87 | GATATCCAGATGACCCAGAGCCCCAGCAGCCTGAGCGCCAGCGTGGGCGAC |
| | AGAGTGACCATCACCTGTCGGGCCAGCCAGGGCATCCGGAACTACCTGGCCT |
| | GGTATCAGCAGAAGCCCGGCAAGGCCCCCAAGCTGCTGATCTACGCCGCCAG |
| | CACCCTGCAGAGCGGCGTGCCAAGCAGATTCAGCGGCAGCGGCTCCGGCAC |
| | CGACTTCACCCTGACCATCAGCAGCCTGCAGCCCGAGGACGTGGCCACCTAC |
| | TACTGCCAGCGGTACAACAGAGCCCCCTACACCTTCGGCCAGGGCACCAAGG |
| | TGGAAATCAAG |
| 88 | CGGGCCAGCCAGGGCATCCGGAACTACCTGGCC |
| 89 | GCCGCCAGCACCCTGCAGAGC |
| 90 | CAGCGGTACAACAGAGCCCCCTACACC |
| 91 | CAGCTACTATTTAATAAAACAAAATCTGTAGAATTCACGTTTTGTAATGACACTG |
| | TCGTCATTCCATGCTTTGTTACTAATATGGAGGCACAAAACACTACTGAAGTAT |
| | ACGTAAAGTGGAAATTTAAAGGAAGAGATATTTACACCTTTGATGGAGCTCTAA |
| | ACAAGTCCACTGTCCCCACTGACTTTAGTAGTGCAAAAATTGAAGTCTCACAAT |
| | TACTAAAAGGAGATGCCTCTTTGAAGATGGATAAGAGTGATGCTGTCTCACAC |
| | ACAGGAAACTACACTTGTGAAGTAACAGAATTAACCAGAGAAGGTGAAACGAT |
| | CATCGAGCTAAAATATCGTGTTGTTTCATGGTTTTCTCCAAATGAAAATGAGGT |
| | GCAATTGGTGGAAAGCGGCGGAGGACTGGTGCAGCCCGGCAGAAGCCTGAG |
| | ACTGAGCTGCGCCGCCAGCGGCTTCACCTTCGACGACTACGCCATGCACTGG |
| | GTCCGCCAGGCCCCTGGCAAGGGACTGGAATGGGTGTCCGCCATCACCTGG |
| | AACAGCGGCCACATCGACTACGCCGACAGCGTGGAAGGCCGGTTCACCATCA |
| | GCCGGGACAACGCCAAGAACAGCCTGTACCTGCAGATGAACAGCCTGCGGGC |
| | CGAGGACACCGCCGTGTACTACTGCGCCAAGGTGTCCTACCTGAGCACCGCC |
| | AGCAGCCTGGACTACTGGGGCCAGGGCACACTGGTCACAGTCAGCTCA |
| 92 | CAGCTACTATTTAATAAAACAAAATCTGTAGAATTCACGTTTTGTAATGACACTG |
| | TCGTCATTCCATGCTTTGTTACTAATATGGAGGCACAAAACACTACTGAAGTAT |
| | ACGTAAAGTGGAAATTTAAAGGAAGAGATATTTACACCTTTGATGGAGCTCTAA |
| | ACAAGTCCACTGTCCCCACTGACTTTAGTAGTGCAAAAATTGAAGTCTCACAAT |
| | TACTAAAAGGAGATGCCTCTTTGAAGATGGATAAGAGTGATGCTGTCTCACAC |
| | ACAGGAAACTACACTTGTGAAGTAACAGAATTAACCAGAGAAGGTGAAACGAT |
| | CATCGAGCTAAAATATCGTGTTGTTTCATGGTTTTCTCCAAATGAAAATGATATC |
| | CAGATGACCCAGAGCCCCAGCAGCCTGAGCGCCAGCGTGGGCGACAGAGTG |
| | ACCATCACCTGTCGGGCCAGCCAGGGCATCCGGAACTACCTGGCCTGGTATC |
| | AGCAGAAGCCCGGCAAGGCCCCCAAGCTGCTGATCTACGCCGCCAGCACCCT |
| | GCAGAGCGGCGTGCCAAGCAGATTCAGCGGCAGCGGCTCCGGCACCGACTT |
| | CACCCTGACCATCAGCAGCCTGCAGCCCGAGGACGTGGCCACCTACTACTGC |
| | CAGCGGTACAACAGAGCCCCCTACACCTTCGGCCAGGGCACCAAGGTGGAAA |
| | TCAAG |
| 93 | CAGCTACTATTTAATAAAACAAAATCTGTAGAATTCACGTTTTGTAATGACACTG |
| | TCGTCATTCCATGCTTTGTTACTAATATGGAGGCACAAAACACTACTGAAGTAT |
| | ACGTAAAGTGGAAATTTAAAGGAAGAGATATTTACACCTTTGATGGAGCTCTAA |
| | ACAAGTCCACTGTCCCCACTGACTTTAGTAGTGCAAAAATTGAAGTCTCACAAT |
| | TACTAAAAGGAGATGCCTCTTTGAAGATGGATAAGAGTGATGCTGTCTCACAC |
| | ACAGGAAACTACACTTGTGAAGTAACAGAATTAACCAGAGAAGGTGAAACGAT |
| | CATCGAGCTAAAATATCGTGTTGTTTCATGGTTTTCTCCAAATGAAAATGGAGG |
| | TGGTGGATCTGGAGGTGGAGGATCCGAGGTCCAATTGGTGGAAAGCGGCGG |
| | AGGACTGGTGCAGCCCGGCAGAAGCCTGAGACTGAGCTGCGCCGCCAGCGG |
| | CTTCACCTTCGACGACTACGCCATGCACTGGGTCCGCCAGGCCCCTGGCAAG |
| | GGACTGGAATGGGTGTCCGCCATCACCTGGAACAGCGGCCACATCGACTACG |
| | CCGACAGCGTGGAAGGCCGGTTCACCATCAGCCGGGACAACGCCAAGAACA |
| | GCCTGTACCTGCAGATGAACAGCCTGCGGGCCGAGGACACCGCCGTGTACTA |
| | CTGCGCCAAGGTGTCCTACCTGAGCACCGCCAGCAGCCTGGACTACTGGGGC |
| | CAGGGCACACTGGTCACAGTCAGCTCA |
| 94 | CAGCTACTATTTAATAAAACAAAATCTGTAGAATTCACGTTTTGTAATGACACTG |
| | TCGTCATTCCATGCTTTGTTACTAATATGGAGGCACAAAACACTACTGAAGTAT |
| | ACGTAAAGTGGAAATTTAAAGGAAGAGATATTTACACCTTTGATGGAGCTCTAA |
| | ACAAGTCCACTGTCCCCACTGACTTTAGTAGTGCAAAAATTGAAGTCTCACAAT |
| | TACTAAAAGGAGATGCCTCTTTGAAGATGGATAAGAGTGATGCTGTCTCACAC |
| | ACAGGAAACTACACTTGTGAAGTAACAGAATTAACCAGAGAAGGTGAAACGAT |
| | CATCGAGCTAAAATATCGTGTTGTTTCATGGTTTTCTCCAAATGAAAATGGAGG |
| | TGGTGGATCTGGAGGTGGAGGATCCGATATCCAGATGACCCAGAGCCCCAGC |
| | AGCCTGAGCGCCAGCGTGGGCGACAGAGTGACCATCACCTGTCGGGCCAGC |
| | CAGGGCATCCGGAACTACCTGGCCTGGTATCAGCAGAAGCCCGGCAAGGCCC |
| | CCAAGCTGCTGATCTACGCCGCCAGCACCCTGCAGAGCGGCGTGCCAAGCAG |
| | ATTCAGCGGCAGCGGCTCCGGCACCGACTTCACCCTGACCATCAGCAGCCTG |
| | CAGCCCGAGGACGTGGCCACCTACTACTGCCAGCGGTACAACAGAGCCCCCT |
| | ACACCTTCGGCCAGGGCACCAAGGTGGAAATCAAG |
| 95 | GAGGTCCAATTGGTGGAAAGCGGCGGAGGACTGGTGCAGCCCGGCAGAAGC |
| | CTGAGACTGAGCTGCGCCGCCAGCGGCTTCACCTTCGACGACTACGCCATGC |
| | ACTGGGTCCGCCAGGCCCCTGGCAAGGGACTGGAATGGGTGTCCGCCATCA |
| | CCTGGAACAGCGGCCACATCGACTACGCCGACAGCGTGGAAGGCCGGTTCAC |
| | CATCAGCCGGGACAACGCCAAGAACAGCCTGTACCTGCAGATGAACAGCCTG |
| | CGGGCCGAGGACACCGCCGTGTACTACTGCGCCAAGGTGTCCTACCTGAGCA |
| | CCGCCAGCAGCCTGGACTACTGGGGCCAGGGCACACTGGTCACAGTCAGCTC |
| | AGCCTCCACCAAGGGCCCATCGGTCTTCCCCCTGGCACCCTCCTCCAAGAGC |
| | ACCTCTGGGGGCACAGCGGCCCTGGGCTGCCTGGTCAAGGACTACTTCCCCG |
| | AACCGGTGACGGTGTCGTGGAACTCAGGCGCCCTGACCAGCGGCGTGCACA |
| | CCTTCCCGGCTGTCCTACAGTCCTCAGGACTCTACTCCCTCAGCAGCGTGGTG |
| | ACCGTGCCCTCCAGCAGCTTGGGCACCCAGACCTACATCTGCAACGTGAATC |
| | ACAAGCCCAGCAACACCAAGGTGGACAAGAAAGTTGAGCCCAAATCTTGTGAC |
| | AAAACTCACACATGCCCACCGTGCCCAGCACCTGAACTCCTGGGGGGACCGT |
| | CAGTCTTCCTCTTCCCCCCAAAACCCAAGGACACCCTCATGATCTCCCGGACC |
| | CCTGAGGTCACATGCGTGGTGGTGGACGTGAGCCACGAAGACCCTGAGGTCA |
| | AGTTCAACTGGTACGTGGACGGCGTGGAGGTGCATAATGCCAAGACAAAGCC |
| | GCGGGAGGAGCAGTACAACAGCACGTACCGGGTGGTCAGCGTCCTCACCGT |
| | CCTGCACCAGGACTGGCTGAATGGCAAGGAGTACAAGTGCAAGGTCTCCAAC |
| | AAAGCCCTCCCAGCCCCCATCGAGAAAACCATCTCCAAAGCCAAAGGGCAGC |
| | CCCGAGAACCACAGGTGTACACCCTGCCCCCATCCCGGGATGAGCTGACCAA |
| | GAACCAGGTCAGCCTGACCTGCCTGGTCAAAGGCTTCTATCCCAGCGACATC |
| | GCCGTGGAGTGGGAGAGCAATGGGCAGCCGGAGAACAACTACAAGACCACG |
| | CCTCCCGTGCTGGACTCCGACGGCTCCTTCTTCCTCTACAGCAAGCTCACCGT |
| | GGACAAGAGCAGGTGGCAGCAGGGGAACGTCTTCTCATGCTCCGTGATGCAT |
| | GAGGCTCTGCACAACCACTACACGCAGAAGAGCCTCTCCCTGTCTCCGGGTA |
| | AATGA |
| 96 | GATATCCAGATGACCCAGAGCCCCAGCAGCCTGAGCGCCAGCGTGGGCGAC |
| | AGAGTGACCATCACCTGTCGGGCCAGCCAGGGCATCCGGAACTACCTGGCCT |
| | GGTATCAGCAGAAGCCCGGCAAGGCCCCCAAGCTGCTGATCTACGCCGCCAG |
| | CACCCTGCAGAGCGGCGTGCCAAGCAGATTCAGCGGCAGCGGCTCCGGCAC |
| | CGACTTCACCCTGACCATCAGCAGCCTGCAGCCCGAGGACGTGGCCACCTAC |
| | TACTGCCAGCGGTACAACAGAGCCCCCTACACCTTCGGCCAGGGCACCAAGG |
| | TGGAAATCAAGCGTACGGTGGCCGCTCCCAGCGTGTTCATCTTCCCCCCCAG |
| | CGACGAGCAGCTGAAGAGCGGCACCGCCAGCGTGGTGTGCCTGCTGAACAA |
| | CTTCTACCCCCGGGAGGCCAAGGTGCAGTGGAAGGTGGACAACGCCCTGCA |
| | GAGCGGCAACAGCCAGGAGAGCGTCACCGAGCAGGACAGCAAGGACTCCAC |
| | CTACAGCCTGAGCAGCACCCTGACCCTGAGCAAGGCCGACTACGAGAAGCAT |
| | AAGGTGTACGCCTGCGAGGTGACCCACCAGGGCCTGTCCAGCCCCGTGACCA |
| | AGAGCTTCAACAGGGGCGAGTGC |
| 97 | ATGTGGCCCCTGGTAGCGGCGCTGTTGCTGGGCTCGGCGTGCTGCGGATCA |
| | GCTCAGCTACTATTTAATAAAACAAAATCTGTAGAATTCACGTTTTGTAATGACA |
| | CTGTCGTCATTCCATGCTTTGTTACTAATATGGAGGCACAAAACACTACTGAAG |
| | TATACGTAAAGTGGAAATTTAAAGGAAGAGATATTTACACCTTTGATGGAGCTC |
| | TAAACAAGTCCACTGTCCCCACTGACTTTAGTAGTGCAAAAATTGAAGTCTCAC |
| | AATTACTAAAAGGAGATGCCTCTTTGAAGATGGATAAGAGTGATGCTGTCTCAC |
| | ACACAGGAAACTACACTTGTGAAGTAACAGAATTAACCAGAGAAGGTGAAACG |
| | ATCATCGAGCTAAAATATCGTGTTGTTTCATGGTTTTCTCCAAATGAAAATGGA |
| | GGTGGTGGATCTGGAGGTGGAGGATCCGAGGTGCAATTGGAGCAGAGCGGC |
| | CCTGTGCTGGTGAAGCCCGGCACCAGCATGAAGATCAGCTGCAAGACCAGCG |
| | GCTACAGCTTCACCGGCTACACCATGTCCTGGGTGCGCCAGAGCCACGGCAA |
| | GAGCCTGGAATGGATCGGCCTGATCATCCCCAGCAACGGCGGCACCAACTAC |
| | AACCAGAAGTTCAAGGACAAGGCCAGCCTGACCGTGGACAAGAGCAGCAGCA |
| | CCGCCTACATGGAACTGCTGTCCCTGACCAGCGAGGACAGCGCCGTGTACTA |
| | CTGCGCCAGACCCAGCTACTACGGCAGCCGGAACTACTACGCCATGGACTAC |
| | TGGGGCCAGGGCACCAGCGTGACCGTCAGCTCAGCTAGCACCAAGGGCCCC |
| | AGCGTGTTCCCCCTGGCCCCCAGCAGCAAGAGCACCAGCGGCGGCACAGCC |
| | GCCCTGGGCTGCCTGGTGAAGGACTACTTCCCCGAGCCCGTGACCGTGTCCT |
| | GGAACAGCGGAGCCCTGACCTCCGGCGTGCACACCTTCCCCGCCGTGCTGC |
| | AGAGCAGCGGCCTGTACAGCCTGTCCAGCGTGGTGACAGTGCCCAGCAGCA |
| | GCCTGGGCACCCAGACCTACATCTGCAACGTGAACCACAAGCCCAGCAACAC |
| | CAAGGTGGACAAGAGAGTGGAGCCCAAGAGCTGCGACAAGACCCACACCTGC |
| | CCCCCCTGCCCAGCCCCAGAGGCAGCGGGCGGACCCTCCGTGTTCCTGTTC |
| | CCCCCCAAGCCCAAGGACACCCTGATGATCAGCAGGACCCCCGAGGTGACCT |
| | GCGTGGTGGTGGACGTGAGCCACGAGGACCCAGAGGTGAAGTTCAACTGGTA |
| | CGTGGACGGCGTGGAGGTGCACAACGCCAAGACCAAGCCCAGAGAGGAGCA |
| | GTACAACAGCACCTACAGGGTGGTGTCCGTGCTGACCGTGCTGCACCAGGAC |
| | TGGCTGAACGGCAAGGAATACAAGTGCAAGGTCTCCAACAAGGCCCTGCCAG |
| | CCCCCATCGAAAAGACCATCAGCAAGGCCAAGGGCCAGCCACGGGAGCCCC |
| | AGGTGTACACCCTGCCCCCCTCCCGGGAGGAGATGACCAAGAACCAGGTGTC |
| | CCTGACCTGTCTGGTGAAGGGCTTCTACCCCAGCGACATCGCCGTGGAGTGG |
| | GAGAGCAACGGCCAGCCCGAGAACAACTACAAGACCACCCCCCCAGTGCTGG |
| | ACAGCGACGGCAGCTTCTTCCTGTACAGCAAGCTGACCGTGGACAAGTCCAG |
| | GTGGCAGCAGGGCAACGTGTTCAGCTGCAGCGTGATGCACGAGGCCCTGCA |
| | CAACCACTACACCCAGAAGAGCCTGAGCCTGTCCCCCGGCAAGTGA |
| 98 | ATGTGGCCCCTGGTAGCGGCGCTGTTGCTGGGCTCGGCGTGCTGCGGATCA |
| | GCTCAGCTACTATTTAATAAAACAAAATCTGTAGAATTCACGTTTTGTAATGACA |
| | CTGTCGTCATTCCATGCTTTGTTACTAATATGGAGGCACAAAACACTACTGAAG |
| | TATACGTAAAGTGGAAATTTAAAGGAAGAGATATTTACACCTTTGATGGAGCTC |
| | TAAACAAGTCCACTGTCCCCACTGACTTTAGTAGTGCAAAAATTGAAGTCTCAC |
| | AATTACTAAAAGGAGATGCCTCTTTGAAGATGGATAAGAGTGATGCTGTCTCAC |
| | ACACAGGAAACTACACTTGTGAAGTAACAGAATTAACCAGAGAAGGTGAAACG |
| | ATCATCGAGCTAAAATATCGTGTTGTTTCATGGTTTTCTCCAAATGAAAATGGA |
| | GGTGGTGGATCTGGAGGTGGAGGATCCGATATCGTGCTGACCCAATCTCCAG |
| | CTTCTTTGGCTGTGTCTCTAGGGCAGAGGGCCACCATCTCCTGCAGGGCCAG |
| | CGAAAGTGTTGATAATTCTGGCTTTAGTTTTATGAACTGGTTCCAACAGAAACC |
| | AGGACAGCCACCCAAACTCCTCATCTATGCTGCATCCAACCAAGGATCCGGG |
| | GTCCCTGCCAGGTTTAGTGGCAGTGGGTCTGAGACAGACTTCAGCCTCAACAT |
| | CCATCCTATGGAGGAGGATGATACTGCAGTGTATTTCTGTCAGCAAAGTAAGG |
| | AGGTTCCTTGGACGTTCGGTGGAGGCACCAAGCTGGAAATCAAGCGTACGGT |
| | GGCCGCTCCCAGCGTGTTCATCTTCCCCCCCAGCGACGAGCAGCTGAAGAGC |
| | GGCACCGCCAGCGTGGTGTGCCTGCTGAACAACTTCTACCCCCGGGAGGCCA |
| | AGGTGCAGTGGAAGGTGGACAACGCCCTGCAGAGCGGCAACAGCCAGGAGA |
| | GCGTCACCGAGCAGGACAGCAAGGACTCCACCTACAGCCTGAGCAGCACCCT |
| | GACCCTGAGCAAGGCCGACTACGAGAAGCATAAGGTGTACGCCTGCGAGGTG |
| | ACCCACCAGGGCCTGTCCAGCCCCGTGACCAAGAGCTTCAACAGGGGCGAGT |
| | GCTGA |
| 99 | GAGGTGCAATTGGAGCAGAGCGGCCCTGTGCTGGTGAAGCCCGGCACCAGC |
| | ATGAAGATCAGCTGCAAGACCAGCGGCTACAGCTTCACCGGCTACACCATGTC |
| | CTGGGTGCGCCAGAGCCACGGCAAGAGCCTGGAATGGATCGGCCTGATCATC |
| | CCCAGCAACGGCGGCACCAACTACAACCAGAAGTTCAAGGACAAGGCCAGCC |
| | TGACCGTGGACAAGAGCAGCAGCACCGCCTACATGGAACTGCTGTCCCTGAC |
| | CAGCGAGGACAGCGCCGTGTACTACTGCGCCAGACCCAGCTACTACGGCAGC |
| | CGGAACTACTACGCCATGGACTACTGGGGCCAGGGCACCAGCGTGACCGTCA |
| | GCTCAGCTAGCACCAAGGGCCCCAGCGTGTTCCCCCTGGCCCCCAGCAGCAA |
| | GAGCACCAGCGGCGGCACAGCCGCCCTGGGCTGCCTGGTGAAGGACTACTT |
| | CCCCGAGCCCGTGACCGTGTCCTGGAACAGCGGAGCCCTGACCTCCGGCGT |
| | GCACACCTTCCCCGCCGTGCTGCAGAGCAGCGGCCTGTACAGCCTGTCCAGC |
| | GTGGTGACAGTGCCCAGCAGCAGCCTGGGCACCCAGACCTACATCTGCAACG |
| | TGAACCACAAGCCCAGCAACACCAAGGTGGACAAGAGAGTGGAGCCCAAGAG |
| | CTGCGACAAGACCCACACCTGCCCCCCCTGCCCAGCCCCAGAGGCAGCGGG |
| | CGGACCCTCCGTGTTCCTGTTCCCCCCCAAGCCCAAGGACACCCTGATGATC |
| | AGCAGGACCCCCGAGGTGACCTGCGTGGTGGTGGACGTGAGCCACGAGGAC |
| | CCAGAGGTGAAGTTCAACTGGTACGTGGACGGCGTGGAGGTGCACAACGCCA |
| | AGACCAAGCCCAGAGAGGAGCAGTACAACAGCACCTACAGGGTGGTGTCCGT |
| | GCTGACCGTGCTGCACCAGGACTGGCTGAACGGCAAGGAATACAAGTGCAAG |
| | GTCTCCAACAAGGCCCTGCCAGCCCCCATCGAAAAGACCATCAGCAAGGCCA |
| | AGGGCCAGCCACGGGAGCCCCAGGTGTACACCCTGCCCCCCTCCCGGGAGG |
| | AGATGACCAAGAACCAGGTGTCCCTGACCTGTCTGGTGAAGGGCTTCTACCC |
| | CAGCGACATCGCCGTGGAGTGGGAGAGCAACGGCCAGCCCGAGAACAACTA |
| | CAAGACCACCCCCCCAGTGCTGGACAGCGACGGCAGCTTCTTCCTGTACAGC |
| | AAGCTGACCGTGGACAAGTCCAGGTGGCAGCAGGGCAACGTGTTCAGCTGCA |
| | GCGTGATGCACGAGGCCCTGCACAACCACTACACCCAGAAGAGCCTGAGCCT |
| | GTCCCCCGGCAAG |
| 100 | GATATCGTGCTGACCCAATCTCCAGCTTCTTTGGCTGTGTCTCTAGGGCAGAG |
| | GGCCACCATCTCCTGCAGGGCCAGCGAAAGTGTTGATAATTCTGGCTTTAGTT |
| | TTATGAACTGGTTCCAACAGAAACCAGGACAGCCACCCAAACTCCTCATCTAT |
| | GCTGCATCCAACCAAGGATCCGGGGTCCCTGCCAGGTTTAGTGGCAGTGGGT |
| | CTGAGACAGACTTCAGCCTCAACATCCATCCTATGGAGGAGGATGATACTGCA |
| | GTGTATTTCTGTCAGCAAAGTAAGGAGGTTCCTTGGACGTTCGGTGGAGGCAC |
| | CAAGCTGGAAATCAAGCGTACGGTGGCCGCTCCCAGCGTGTTCATCTTCCCC |
| | CCCAGCGACGAGCAGCTGAAGAGCGGCACCGCCAGCGTGGTGTGCCTGCTG |
| | AACAACTTCTACCCCCGGGAGGCCAAGGTGCAGTGGAAGGTGGACAACGCCC |
| | TGCAGAGCGGCAACAGCCAGGAGAGCGTCACCGAGCAGGACAGCAAGGACT |
| | CCACCTACAGCCTGAGCAGCACCCTGACCCTGAGCAAGGCCGACTACGAGAA |
| | GCATAAGGTGTACGCCTGCGAGGTGACCCACCAGGGCCTGTCCAGCCCCGTG |
| | ACCAAGAGCTTCAACAGGGGCGAGTGC |
| 101 | GAGGTGCAATTGGAGCAGAGCGGCCCTGTGCTGGTGAAGCCCGGCACCAGC |
| | ATGAAGATCAGCTGCAAGACCAGCGGCTACAGCTTCACCGGCTACACCATGTC |
| | CTGGGTGCGCCAGAGCCACGGCAAGAGCCTGGAATGGATCGGCCTGATCATC |
| | CCCAGCAACGGCGGCACCAACTACAACCAGAAGTTCAAGGACAAGGCCAGCC |
| | TGACCGTGGACAAGAGCAGCAGCACCGCCTACATGGAACTGCTGTCCCTGAC |
| | CAGCGAGGACAGCGCCGTGTACTACTGCGCCAGACCCAGCTACTACGGCAGC |
| | CGGAACTACTACGCCATGGACTACTGGGGCCAGGGCACCAGCGTGACCGTCA |
| | GC |
| 102 | GGCTACACCATGTCC |
| 103 | CTGATCATCCCCAGCAACGGCGGCACCAACTACAACCAGAAGTTCAAGGAC |
| 104 | CCCAGCTACTACGGCAGCCGGAACTACTACGCCATGGACTAC |
| 105 | GATATCGTGCTGACCCAATCTCCAGCTTCTTTGGCTGTGTCTCTAGGGCAGAG |
| | GGCCACCATCTCCTGCAGGGCCAGCGAAAGTGTTGATAATTCTGGCTTTAGTT |
| | TTATGAACTGGTTCCAACAGAAACCAGGACAGCCACCCAAACTCCTCATCTAT |
| | GCTGCATCCAACCAAGGATCCGGGGTCCCTGCCAGGTTTAGTGGCAGTGGGT |
| | CTGAGACAGACTTCAGCCTCAACATCCATCCTATGGAGGAGGATGATACTGCA |
| | GTGTATTTCTGTCAGCAAAGTAAGGAGGTTCCTTGGACGTTCGGTGGAGGCAC |
| | CAAGCTGGAAATCAAG |
| 106 | AGGGCCAGCGAAAGTGTTGATAATTCTGGCTTTAGTTTTATGAAC |
| 107 | GCTGCATCCAACCAAGGATCC |
| 108 | CAGCAAAGTAAGGAGGTTCCTTGGACG |
| 109 | CAGCTACTATTTAATAAAACAAAATCTGTAGAATTCACGTTTTGTAATGACACTG |
| | TCGTCATTCCATGCTTTGTTACTAATATGGAGGCACAAAACACTACTGAAGTAT |
| | ACGTAAAGTGGAAATTTAAAGGAAGAGATATTTACACCTTTGATGGAGCTCTAA |
| | ACAAGTCCACTGTCCCCACTGACTTTAGTAGTGCAAAAATTGAAGTCTCACAAT |
| | TACTAAAAGGAGATGCCTCTTTGAAGATGGATAAGAGTGATGCTGTCTCACAC |
| | ACAGGAAACTACACTTGTGAAGTAACAGAATTAACCAGAGAAGGTGAAACGAT |
| | CATCGAGCTAAAATATCGTGTTGTTTCATGGTTTTCTCCAAATGAAAATGGAGG |
| | TGGTGGATCTGGAGGTGGAGGATCCGAGGTGCAATTGGAGCAGAGCGGCCC |
| | TGTGCTGGTGAAGCCCGGCACCAGCATGAAGATCAGCTGCAAGACCAGCGGC |
| | TACAGCTTCACCGGCTACACCATGTCCTGGGTGCGCCAGAGCCACGGCAAGA |
| | GCCTGGAATGGATCGGCCTGATCATCCCCAGCAACGGCGGCACCAACTACAA |
| | CCAGAAGTTCAAGGACAAGGCCAGCCTGACCGTGGACAAGAGCAGCAGCACC |
| | GCCTACATGGAACTGCTGTCCCTGACCAGCGAGGACAGCGCCGTGTACTACT |
| | GCGCCAGACCCAGCTACTACGGCAGCCGGAACTACTACGCCATGGACTACTG |
| | GGGCCAGGGCACCAGCGTGACCGTCAGCTCA |
| 110 | CAGCTACTATTTAATAAAACAAAATCTGTAGAATTCACGTTTTGTAATGACACTG |
| | TCGTCATTCCATGCTTTGTTACTAATATGGAGGCACAAAACACTACTGAAGTAT |
| | ACGTAAAGTGGAAATTTAAAGGAAGAGATATTTACACCTTTGATGGAGCTCTAA |
| | ACAAGTCCACTGTCCCCACTGACTTTAGTAGTGCAAAAATTGAAGTCTCACAAT |
| | TACTAAAAGGAGATGCCTCTTTGAAGATGGATAAGAGTGATGCTGTCTCACAC |
| | ACAGGAAACTACACTTGTGAAGTAACAGAATTAACCAGAGAAGGTGAAACGAT |
| | CATCGAGCTAAAATATCGTGTTGTTTCATGGTTTTCTCCAAATGAAAATGGAGG |
| | TGGTGGATCTGGAGGTGGAGGATCCGATATCGTGCTGACCCAATCTCCAGCTT |
| | CTTTGGCTGTGTCTCTAGGGCAGAGGGCCACCATCTCCTGCAGGGCCAGCGA |
| | AAGTGTTGATAATTCTGGCTTTAGTTTTATGAACTGGTTCCAACAGAAACCAGG |
| | ACAGCCACCCAAACTCCTCATCTATGCTGCATCCAACCAAGGATCCGGGGTCC |
| | CTGCCAGGTTTAGTGGCAGTGGGTCTGAGACAGACTTCAGCCTCAACATCCAT |
| | CCTATGGAGGAGGATGATACTGCAGTGTATTTCTGTCAGCAAAGTAAGGAGGT |
| | TCCTTGGACGTTCGGTGGAGGCACCAAGCTGGAAATCAAG |
| 111 | TCAGCCTCCACCAAGGGCCCATCGGTCTTCCCCCTGGCACCCTCCTCCAAGA |
| | GCACCTCTGGGGGCACAGCGGCCCTGGGCTGCCTGGTCAAGGACTACTTCCC |
| | CGAACCGGTGACGGTGTCGTGGAACTCAGGCGCCCTGACCAGCGGCGTGCA |
| | CACCTTCCCGGCTGTCCTACAGTCCTCAGGACTCTACTCCCTCAGCAGCGTGG |
| | TGACCGTGCCCTCCAGCAGCTTGGGCACCCAGACCTACATCTGCAACGTGAA |
| | TCACAAGCCCAGCAACACCAAGGTGGACAAGAAAGTT |
| 112 | CGTACGGTGGCCGCTCCCAGCGTGTTCATCTTCCCCCCCAGCGACGAGCAGC |
| | TGAAGAGCGGCACCGCCAGCGTGGTGTGCCTGCTGAACAACTTCTACCCCCG |
| | GGAGGCCAAGGTGCAGTGGAAGGTGGACAACGCCCTGCAGAGCGGCAACAG |
| | CCAGGAGAGCGTCACCGAGCAGGACAGCAAGGACTCCACCTACAGCCTGAGC |
| | AGCACCCTGACCCTGAGCAAGGCCGACTACGAGAAGCATAAGGTGTACGCCT |
| | GCGAGGTGACCCACCAGGGCCTGTCCAGCCCCGTGACCAAGAGCTTCAACAG |
| | GGGCGAGTGC |
| 113 | GAGCCCAAATCTTGTGACAAAACTCACACATGCCCACCGTGCCCAGCACCTGA |
| | ACTCCTGGGGGGACCGTCAGTCTTCCTCTTCCCCCCAAAACCCAAGGACACC |
| | CTCATGATCTCCCGGACCCCTGAGGTCACATGCGTGGTGGTGGACGTGAGCC |
| | ACGAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGGGTG |
| | GTCAGCGTCCTCACCGTCCTGCACCAGGACTGGCTGAATGGCAAGGAGTACA |
| | AGTGCAAGGTCTCCAACAAAGCCCTCCCAGCCCCCATCGAGAAAACCATCTCC |
| | AAAGCCAAAGGGCAGCCCCGAGAACCACAGGTGTACACCCTGCCCCCATCCC |
| | GGGATGAGCTGACCAAGAACCAGGTCAGCCTGACCTGCCTGGTCAAAGGCTT |
| | CTATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCCTCT |
| | ACAGCAAGCTCACCGTGGACAAGAGCAGGTGGCAGCAGGGGAACGTCTTCTC |
| | ATGCTCCGTGATGCATGAGGCTCTGCACAACCACTACACGCAGAAGAGCCTCT |
| | CCCTGTCTCCGGGTAAA |
| 114 | CAGCTACTATTTAATAAAACAAAATCTGTAGAATTCACGTTTTGTAATGACACTG |
| | TCGTCATTCCATGCTTTGTTACTAATATGGAGGCACAAAACACTACTGAAGTAT |
| | ACGTAAAGTGGAAATTTAAAGGAAGAGATATTTACACCTTTGATGGAGCTCTAA |
| | ACAAGTCCACTGTCCCCACTGACTTTAGTAGTGCAAAAATTGAAGTCTCACAAT |
| | TACTAAAAGGAGATGCCTCTTTGAAGATGGATAAGAGTGATGCTGTCTCACAC |
| | ACAGGAAACTACACTTGTGAAGTAACAGAATTAACCAGAGAAGGTGAAACGAT |
| | CATCGAGCTAAAATATCGTGTTGTTTCA |
| 115 | MPVPASWPHPPGPFLLLTLLLGLTEVAGEEELQMIQPEKLLLVTVGKTATLHCTVT |
| | SLLPVGPVLWFRGVGPGRELIYNQKEGHFPRVTTVSDLTKRNNMDFSIRISSITPA |
| | DVGTYYCVKFRKGSPENVEFKSGPGTEMALGAKPSAPVVLGPAARTTPEHTVSF |
| | TCESHGFSPRDITLKWFKNGNELSDFQTNVDPTGQSVAYSIRSTARVVLDPWDVR |
| | SQVICEVAHVTLQGDPLRGTANLSEAIRVPPTLEVTQQPMRVGNQVNVTCQVRKF |
| | YPQSLQLTWSENGNVCQRETASTLTENKDGTYNWTSWFLVNISDQRDDVVLTCQ |
| | VKHDGQLAVSKRLALEVTVHQKDQSSDATPGPASSLTALLLIAVLLGPIYVPWKQK |
| | T |
| 116 | LEPKSCDKTHTCPPCPAPELLGGPSVFLFPPKPKDTLMISRTPEVTCVVVDVSHED |
| | PEVKFNWYVDGVEVHNAKTKPREEQYASTYRWSVLTVLHQDWLNGKEYKCKVS |
| | NKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALH |
| | NHYTQKSLSLSPGK |
| 117 | QLLFNKTKSVEFTFCNDTVVIPCFVTNMEAQNTTEVYVKWKFKGRDIYTFDGALNK |
| | STVPTDFSSAKIEVSQLLKGDASLKMDKSDAVSHTGNYTCEVTELTREGETIIELKY |
| | RVVSWFSPNENLEPKSCDKTHTCPPCPAPELLGGPSVFLFPPKPKDTLMISRTPE |
| | VTCVVVDVSHEDPEVKFNWYVDGVEVHNAKTKPREEQYASTYRVVSVLTVLHQD |
| | WLNGKEYKCKVSNKALPAPIEKTISKAKGQPREPQVYTLPPSREEMTKNQVSLTC |
| | LVKGFYPSDIAVEWESNGQPENNYKTTPPVLDSDGSFFLYSKLTVDKSRWQQGN |
| | VFSCSVMHEALHNHYTQKSLSLSPGK |
| 118 | CTCGAGCCCAAATCTTGTGACAAAACTCACACATGCCCACCGTGCCCAGCACC |
| | TGAACTCCTGGGGGGACCGTCAGTCTTCCTCTTCCCCCCAAAACCCAAGGACA |
| | CCCTCATGATCTCCCGGACCCCTGAGGTCACATGCGTGGTGGTGGACGTGAG |
| | CCACGAAGACCCTGAGGTCAAGTTCAACTGGTACGTGGACGGCGTGGAGGTG |
| | CATAATGCCAAGACAAAGCCGCGGGAGGAGCAGTACGCCAGCACGTACCGG |
| | GTGGTCAGCGTCCTCACCGTCCTGCACCAGGACTGGCTGAATGGCAAGGAGT |
| | ACAAGTGCAAGGTCTCCAACAAAGCCCTCCCAGCCCCCATCGAGAAAACCATC |
| | TCCAAAGCCAAAGGGCAGCCCCGAGAACCACAGGTGTACACCCTGCCCCCAT |
| | CCCGGGAGGAGATGACCAAGAACCAGGTCAGCCTGACCTGCCTGGTCAAAGG |
| | CTTCTATCCCAGCGACATCGCCGTGGAGTGGGAGAGCAATGGGCAGCCGGAG |
| | AACAACTACAAGACCACGCCTCCCGTGCTGGACTCCGACGGCTCCTTCTTCCT |
| | CTACAGCAAGCTCACCGTGGACAAGAGCAGGTGGCAGCAGGGGAACGTCTTC |
| | TCATGCTCCGTGATGCATGAGGCTCTGCACAACCACTACACGCAGAAGAGCCT |
| | CTCCCTGTCTCCGGGTAAA |
| 119 | ATGTGGCCCCTGGTAGCGGCGCTGTTGCTGGGCTCGGCGTGCTGCGGATCA |
| | GCTCAGCTACTATTTAATAAAACAAAATCTGTAGAATTCACGTTTTGTAATGACA |
| | CTGTCGTCATTCCATGCTTTGTTACTAATATGGAGGCACAAAACACTACTGAAG |
| | TATACGTAAAGTGGAAATTTAAAGGAAGAGATATTTACACCTTTGATGGAGCTC |
| | TAAACAAGTCCACTGTCCCCACTGACTTTAGTAGTGCAAAAATTGAAGTCTCAC |
| | AATTACTAAAAGGAGATGCCTCTTTGAAGATGGATAAGAGTGATGCTGTCTCAC |
| | ACACAGGAAACTACACTTGTGAAGTAACAGAATTAACCAGAGAAGGTGAAACG |
| | ATCATCGAGCTAAAATATCGTGTTGTTTCATGGTTTTCTCCAAATGAAAATCTC |
| | GAGCCCAAATCTTGTGACAAAACTCACACATGCCCACCGTGCCCAGCACCTGA |
| | ACTCCTGGGGGGACCGTCAGTCTTCCTCTTCCCCCCAAAACCCAAGGACACC |
| | CTCATGATCTCCCGGACCCCTGAGGTCACATGCGTGGTGGTGGACGTGAGCC |
| | ACGAAGACCCTGAGGTCAAGTTCAACTGGTACGTGGACGGCGTGGAGGTGCA |
| | TAATGCCAAGACAAAGCCGCGGGAGGAGCAGTACGCCAGCACGTACCGGGT |
| | GGTCAGCGTCCTCACCGTCCTGCACCAGGACTGGCTGAATGGCAAGGAGTAC |
| | AAGTGCAAGGTCTCCAACAAAGCCCTCCCAGCCCCCATCGAGAAAACCATCTC |
| | CAAAGCCAAAGGGCAGCCCCGAGAACCACAGGTGTACACCCTGCCCCCATCC |
| | CGGGAGGAGATGACCAAGAACCAGGTCAGCCTGACCTGCCTGGTCAAAGGCT |
| | TCTATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCCTCT |
| | ACAGCAAGCTCACCGTGGACAAGAGCAGGTGGCAGCAGGGGAACGTCTTCTC |
| | ATGCTCCGTGATGCATGAGGCTCTGCACAACCACTACACGCAGAAGAGCCTCT |
| | CCCTGTCTCCGGGTAAATGA |
| 120 | EPKSCGGGGSGGGGS |
| 121 | GAGCCCAAGAGCTGCGGCGGCGGCGGCTCCGGCGGCGGCGGATCC |
| 122 | EPKSCDKTHTCPPCPAPELLGGPSVFLFPPKPKDTLMISRTPEVTCVVVDVSHED |
| | PEVKFNWYVDGVEVHNAKTKPREEQYNSTYRVVSVLTVLHQDWLNGKEYKCKVS |
| | NKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALH |
| | NHYTQKSLSLSPGK |
| 123 | GAGCCCAAGAGCTGCGACAAGACCCACACCTGCCCCCCCTGCCCAGCCCCA |
| | GAGCTGCTGGGCGGACCCTCCGTGTTCCTGTTCCCCCCCAAGCCCAAGGACA |
| | CCCTGATGATCAGCAGGACCCCCGAGGTGACCTGCGTGGTGGTGGACGTGA |
| | GCCACGAGGACCCAGAGGTGAAGTTCAACTGGTACGTGGACGGCGTGGAGG |
| | TGCACAACGCCAAGACCAAGCCCAGAGAGGAGCAGTACAACAGCACCTACAG |
| | GGTGGTGTCCGTGCTGACCGTGCTGCACCAGGACTGGCTGAACGGCAAGGAA |
| | TACAAGTGCAAGGTCTCCAACAAGGCCCTGCCAGCCCCCATCGAAAAGACCAT |
| | CAGCAAGGCCAAGGGCCAGCCACGGGAGCCCCAGGTGTACACCCTGCCCCC |
| | CTCCCGGGAGGAGATGACCAAGAACCAGGTGTCCCTGACCTGTCTGGTGAAG |
| | GGCTTCTACCCCAGCGACATCGCCGTGGAGTGGGAGAGCAACGGCCAGCCC |
| | GAGAACAACTACAAGACCACCCCCCCAGTGCTGGACAGCGACGGCAGCTTCT |
| | TCCTGTACAGCAAGCTGACCGTGGACAAGTCCAGGTGGCAGCAGGGCAACGT |
| | GTTCAGCTGCAGCGTGATGCACGAGGCCCTGCACAACCACTACACCCAGAAG |
| | AGCCTGAGCCTGTCCCCCGGCAAG |
| 124 | AGCGCTAGCACCAAGGGCCCCAGCGTGTTCCCCCTGGCCCCCAGCAGCAAG |
| | AGCACCAGCGGCGGCACAGCCGCCCTGGGCTGCCTGGTGAAGGACTACTTC |
| | CCCGAGCCCGTGACCGTGTCCTGGAACAGCGGAGCCCTGACCTCCGGCGTG |
| | CACACCTTCCCCGCCGTGCTGCAGAGCAGCGGCCTGTACAGCCTGTCCAGCG |
| | TGGTGACAGTGCCCAGCAGCAGCCTGGGCACCCAGACCTACATCTGCAACGT |
| | GAACCACAAGCCCAGCAACACCAAGGTGGACAAGAGAGTG |
| 125 | GGCGGCGGCGGCTCCGGCGGCGGCGGATCC |
| 126 | GGAGGTGGTGGATCTGGAGGTGGAGGATCC |
| 127 | GAGGTGCAATTGGTGGAAAGCGGCGGAGGACTGGTGCAGCCCGGCAGAAGC |
| | CTGAGACTGAGCTGCGCCGCCAGCGGCTTCACCTTCGACGACTACGCCATGC |
| | ACTGGGTCCGCCAGGCCCCTGGCAAGGGACTGGAATGGGTGTCCGCCATCA |
| | CCTGGAACAGCGGCCACATCGACTACGCCGACAGCGTGGAAGGCCGGTTCAC |
| | CATCAGCCGGGACAACGCCAAGAACAGCCTGTACCTGCAGATGAACAGCCTG |
| | CGGGCCGAGGACACCGCCGTGTACTACTGCGCCAAGGTGTCCTACCTGAGCA |
| | CCGCCAGCAGCCTGGACTACTGGGGCCAGGGCACACTGGTCACAGTCAGC |

1. A soluble protein, comprising a complex of two heterodimers, wherein each heterodimer essentially consists of:
(i) a first single chain polypeptide comprising:
(a) an antibody heavy chain sequence having VH, CH1, CH2, and CH3 regions; and
(b) a monovalent region of a mammalian binding molecule fused to the VH region; and
(ii) a second single chain polypeptide comprising:
(c) an antibody light chain sequence having a VL and CL region; and
(d) a monovalent region of a mammalian binding molecule fused to the VL region;
characterised in that each pair of VH and VL CDR sequences has specificity for an antigen, such that the total valency of said soluble protein is six.

2. The soluble protein as claimed in claim 1 wherein the protein has binding specificity for one, two or three antigens.

3. The soluble protein as claimed in claim 1 wherein the regions of the mammalian binding molecule comprised within said first and second single chain polypeptides are the same.

4. The soluble protein as claimed in claim 1, wherein each region of the mammalian binding molecule and each pair of VH and VL CDR sequences have binding specificity for the same single antigen.

5. The soluble protein as claimed in claim 1, wherein the regions of the mammalian binding molecule can bind a first epitope on the antigen, and each pair of VH and VL CDR sequences can bind a second epitope on the same antigen.

6. The soluble protein as claimed in claim 1, wherein the regions of the mammalian binding molecule and each pair of VH and VL CDR sequences can bind the same epitope on the same antigen.

7. The soluble protein as claimed in claim 1, said protein having binding specificity for two antigens, wherein each region of the mammalian binding molecule has binding specificity for a first antigen, and each pair of VH and VL CDR sequences has binding specificity for a second antigen.

8. The soluble protein as claimed in claim 1, wherein the mammalian binding molecule comprised within said first and second single chain polypeptides is different.

9. The soluble protein as claimed in claim 8, said protein having binding specificity for two antigens, wherein the regions of the mammalian binding molecule comprised within the first single polypeptide chain have binding specificity for a first antigen, and the regions of the mammalian binding molecule comprised within the second single polypeptide chain have binding specificity for a second antigen, and each pair of VH and VL CDR sequences has binding specificity for either the first or second antigen.

10. The soluble protein as claimed in claim 1, said protein having binding specificity for three antigens, wherein the regions of the mammalian binding molecule comprised within the first single polypeptide chain have binding specificity for a first antigen, the regions of the mammalian binding molecule comprised within the second single polypeptide chain have binding specificity for a second antigen, and each pair of VH and VL CDR sequences has binding specificity for a third antigen.

11. The soluble protein as claimed in claim 1, wherein said mammalian binding molecule is a protein, cytokine, growth factor, hormone, signaling protein, inflammatory mediator, low molecular weight compound, ligand, cell surface receptor, or fragment thereof.

12. The soluble protein as claimed in claim 11, wherein said mammalian binding molecule is an extracellular domain of a monomeric or homopolymeric cell surface receptor.

13. The soluble protein as claimed in claim 12, wherein said mammalian monomeric or homopolymeric cell surface receptor comprises an IgSF domain.

14. The soluble protein as claimed in claim 12, wherein said mammalian binding molecule comprises a SIRPalpha binding domain.

15. The soluble protein as claimed in claim 14, wherein said SIRPα binding domain is selected from the group consisting of:
(i) an extracellular domain of the human cell surface receptor CD47;
(ii) an extracellular domain derived of SEQ ID NO:2;
(iii) a polypeptide of SEQ ID NO:3, SEQ ID NO:4, SEQ ID NO:5, SEQ ID NO:57 or a fragment thereof retaining SIRPα binding properties; and,
(iv) a variant polypeptide of SEQ ID NO:3, SEQ ID NO:4, SEQ ID NO:5, SEQ ID NO:57 or said fragment, having at least 60, 70, 80, 90, 95, 96, 97, 98, or 99 percent sequence identity, and retaining SIRPα binding properties.

16. The soluble protein as claimed in claim 14, wherein two or more SIRPα binding domains comprised within said first and second single polypeptide chains share at least 60, 70, 80, 90, 95, 96, 97, 98, 99, or 99.5% percent sequence identity with each other.

17. The soluble protein as claimed in claim 14 wherein two or more SIRPα binding domains have identical amino acid sequences.

18. The soluble protein as claimed in claim 14, wherein the SIRPα binding domains within each heterodimer have identical amino acid sequences.

19. The soluble protein as claimed in claim 14, wherein the SIRPα binding domain is an extracellular domain of the human cell surface receptor CD47 having an amino acid sequence selected from the group consisting of: SEQ ID NO:3, SEQ ID NO:4, SEQ ID NO:5 and SEQ ID NO:57.

20. The soluble protein as claimed in claim 15, comprising a complex of two heterodimers, wherein each heterodimer essentially consists of:
(i) a first single chain polypeptide comprising:
(a) an antibody heavy chain sequence having VH, CH1, CH2, and CH3 regions; and
(b) a monovalent region of an extracellular domain of CD47, the carboxyl-terminus of said CD47 region being fused to the N-terminus of the VH region; and
(ii) a second single chain polypeptide comprising:
(c) an antibody light chain sequence having a VL and CL region; and
(d) a monovalent region of an extracellular domain of CD47, the carboxyl-terminus of said CD47 region being fused to the N-terminus of the VL region.

21. The soluble protein as claimed in claim 20, wherein said region of an extracellular domain of CD47 is SEQ ID NO:3 or SEQ ID NO:57.

22. The soluble protein as claimed in claim 1, wherein the VH and VL CDR sequences have binding specificity for TNFalpha, cyclosporin A, or epitopes derived therefrom.

23. The soluble protein as claimed in claim 14, which dissociates from binding to human SIRPalpha with a koff (kd1) of 0.05 [1/s] or less, as measured in a BiaCORE assay, applying a bivalent kinetic fitting model.

24. The soluble protein as claimed in claim 14, which inhibits the Staphylococcus aureus Cowan strain particles stimulated release of proinflammatory cytokines of in vitro generated monocyte-derived dendritic cells.

25. The soluble protein of claim 24, which inhibits the Staphylococcus aureus Cowan strain particle-stimulated release of proinflammatory cytokines in in vitro generated monocyte-derived dendritic cells dendritic cells, with an IC₅₀ of 0.1 nM or less, as measured in a dendritic cell cytokine release assay.

26. The soluble protein as claimed in claim 1, wherein said first and second single chain polypeptides of each heterodimer are covalently bound by a disulfide bridge.

27. The soluble protein as claimed in claim 1, wherein said first single chain polypeptide of each heterodimer comprises the hinge region of an immunoglobulin constant part, and said two heterodimers are stably associated with each other by a disulfide bridge at said hinge region.

28. The soluble protein as claimed in claim 1, wherein each region of said mammalian binding molecule is fused to its respective VH or VL sequence in the absence of peptide linkers.

29. The soluble protein as claimed in claim 1, wherein each region of said mammalian binding molecule is fused to its respective VH or VL sequence via peptide linkers.

30. The soluble protein as claimed in claim 29, wherein said peptide linker comprises 5 to 20 amino acids.

31. The soluble protein as claimed in claim 29, wherein said peptide linker is a polymer of glycine and serine amino acids, preferably of (GGGGS)ₙ, wherein n is any integer between 1 and 4, preferably 2.

32. The soluble protein as claimed in claim 1 wherein the C_{H}1, C_{H}2 and C_{H}3 regions of the antibody are derived from a silent mutant of human IgG1, IgG2, or IgG4 corresponding regions with reduced ADCC effector function.

33. The soluble protein as claimed in claim 1, wherein said heterodimers comprise either:
(i) a first single chain polypeptide of SEQ ID NO:20 and a second single chain polypeptide of SEQ ID NO:21;
(ii) a first single chain polypeptide of SEQ ID NO:22 and a second single chain polypeptide of SEQ ID NO:23; or
(ii) a first single chain polypeptide of SEQ ID NO:40 and a second single chain polypeptide of SEQ ID NO:41.

34. The soluble protein as claimed in claim 1, wherein said first and said second single chain polypeptides have at least 60, 70, 80, 90, 95, 96, 97, 98, or 99 percent sequence identity to the corresponding first and second single chain polypeptides of
(i) SEQ ID NO:20 and SEQ ID NO:21;
(ii) SEQ ID NO:22 and SEQ ID NO:23; or
(ii) SEQ ID NO:40 and SEQ ID NO:41.

35. The soluble protein as claimed in claim 1 comprising:
(i) a heavy chain encoded by a nucleotide sequence of SEQ ID NO:77; and a light chain encoded by a nucleotide sequence of SEQ ID NO:78,
(ii) a heavy chain encoded by a nucleotide sequence of SEQ ID NO:79; and a light chain encoded by a nucleotide sequence of SEQ ID NO:80,
(iii) a heavy chain encoded by a nucleotide sequence of SEQ ID NO:97; and a light chain encoded by a nucleotide sequence of SEQ ID NO:98,

36. A multivalent soluble protein complex comprising two or more soluble proteins as claimed in claim 1, wherein if the protein complex comprises N soluble proteins, the valency is N×6.

37.-41. (canceled)

42. A pharmaceutical composition comprising a soluble protein or protein complex as claimed in claim 1, in combination with one or more pharmaceutically acceptable vehicles.

43. The pharmaceutical composition as claimed in claim 42, additionally comprising at least one other active ingredient.

44. An isolated nucleic acid encoding at least one single chain polypeptide of one heterodimer of the soluble protein as claimed in claim 1.

45. The isolated nucleic acid as claimed in claim 44, or a cloning or expression vector, comprising at least one nucleic acid selected from the group consisting of: SEQ ID NO:77, SEQ ID NO:78, SEQ ID NO:79, SEQ ID NO:80, SEQ ID NO:97, and SEQ ID NO:98.

46. A recombinant host cell suitable for the production of a soluble protein or protein complex as claimed in claim 1, comprising the nucleic acids encoding said first and second single chain polypeptides of said heterodimers of said protein, and optionally, secretion signals.

47. The recombinant host cell as claimed in claim 46, comprising the nucleic acids of SEQ ID NO:77 and SEQ ID NO:78; or SEQ ID NO:79 and SEQ ID NO:80; or SEQ ID NO:97 and SEQ ID NO:98 stably integrated in the genome.

48. The recombinant host cell as claimed in claim 46, wherein said host cell is a mammalian cell line.

49. A process for the production of a soluble protein or protein complex as claimed in claim 1, comprising culturing a recombinant host cell suitable for the production of said soluble protein or protein complex under appropriate conditions for the production of said soluble protein or protein complex, and isolating said protein.


SEQUENCE LISTING (127 sequences)

SEQ ID NO: 1 - protein, 504 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 2 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 3 - protein, 123 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNE

SEQ ID NO: 4 - protein, 124 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNEN

SEQ ID NO: 5 - protein, 124 aa, Homo sapiens
QLLFNKTKSVEFTFGNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNEN

SEQ ID NO: 6 - protein, 233 aa, Homo sapiens
LEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 357 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENLEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 5 aa, Artificial Sequence
  G4S linker
GGGGS

SEQ ID NO: 9 - protein, 10 aa, Artificial Sequence
  G4S G4S linker
GGGGSGGGGS

SEQ ID NO: 10 - protein, 99 aa, Homo sapiens
SASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 11 - protein, 232 aa, Homo sapiens
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 331 aa, Homo sapiens
SASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 107 aa, Homo sapiens [sequence omitted]

SEQ ID NO: 14 - protein, 465 aa, Homo sapiens
QLLFNKTKSVEFTFGNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENGGGGSGGGGSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 241 aa, Homo sapiens
QLLFNKTKSVEFTFGNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 465 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENGGGGSGGGGSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 241 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 567 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSGGGGSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 346 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 575 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 338 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 585 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENGGGGSGGGGSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 348 aa, Homo sapiens [sequence omitted]

SEQ ID NO: 24 - protein, 451 aa, Homo sapiens
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 214 aa, Homo sapiens
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 120 aa, Homo sapiens
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVS

SEQ ID NO: 27 - protein, 5 aa, Homo sapiens
DYAMH

SEQ ID NO: 28 - protein, 17 aa, Homo sapiens
AITWNSGHIDYADSVEG

SEQ ID NO: 29 - protein, 12 aa, Homo sapiens
VSYLSTASSLDY

SEQ ID NO: 30 - protein, 107 aa, Homo sapiens [sequence omitted]

SEQ ID NO: 31 - protein, 11 aa, Homo sapiens
RASQGIRNYLA

SEQ ID NO: 32 - protein, 7 aa, Homo sapiens
AASTLQS

SEQ ID NO: 33 - protein, 9 aa, Homo sapiens
QRYNRAPYT

SEQ ID NO: 34 - protein, 245 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSS

SEQ ID NO: 35 - protein, 231 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIK

SEQ ID NO: 36 - protein, 255 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENGGGGSGGGGSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSS

SEQ ID NO: 37 - protein, 241 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIK

SEQ ID NO: 38 - protein, 451 aa, Homo sapiens
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 214 aa, Homo sapiens; identical to SEQ ID NO: 25

SEQ ID NO: 40 - protein, 587 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENGGGGSGGGGSEVQLEQSGPVLVKPGTSMKISCKTSGYSFTGYTMSWVRQSHGKSLEWIGLIIPSNGGTNYNQKFKDKASLTVDKSSSTAYMELLSLTSEDSAVYYCARPSYYGSRNYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 352 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENGGGGSGGGGSDIVLTQSPASLAVSLGQRATISCRASESVDNSGFSFMNWFQQKPGQPPKLLIYAASNQGSGVPARFSGSGSETDFSLNIHPMEEDDTAVYFCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 453 aa, Homo sapiens
EVQLEQSGPVLVKPGTSMKISCKTSGYSFTGYTMSWVRQSHGKSLEWIGLIIPSNGGTNYNQKFKDKASLTVDKSSSTAYMELLSLTSEDSAVYYCARPSYYGSRNYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 218 aa, Homo sapiens
DIVLTQSPASLAVSLGQRATISCRASESVDNSGFSFMNWFQQKPGQPPKLLIYAASNQGSGVPARFSGSGSETDFSLNIHPMEEDDTAVYFCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 122 aa, Homo sapiens
EVQLEQSGPVLVKPGTSMKISCKTSGYSFTGYTMSWVRQSHGKSLEWIGLIIPSNGGTNYNQKFKDKASLTVDKSSSTAYMELLSLTSEDSAVYYCARPSYYGSRNYYAMDYWGQGTSVTVS

SEQ ID NO: 45 - protein, 5 aa, Homo sapiens
GYTMS

SEQ ID NO: 46 - protein, 17 aa, Homo sapiens
LIIPSNGGTNYNQKFKD

SEQ ID NO: 47 - protein, 14 aa, Homo sapiens
PSYYGSRNYYAMDY

SEQ ID NO: 48 - protein, 111 aa, Homo sapiens
DIVLTQSPASLAVSLGQRATISCRASESVDNSGFSFMNWFQQKPGQPPKLLIYAASNQGSGVPARFSGSGSETDFSLNIHPMEEDDTAVYFCQQSKEVPWTFGGGTKLEIK

SEQ ID NO: 49 - protein, 15 aa, Homo sapiens
RASESVDNSGFSFMN

SEQ ID NO: 50 - protein, 7 aa, Homo sapiens
AASNQGS

SEQ ID NO: 51 - protein, 9 aa, Homo sapiens
QQSKEVPWT

SEQ ID NO: 52 - protein, 257 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENGGGGSGGGGSEVQLEQSGPVLVKPGTSMKISCKTSGYSFTGYTMSWVRQSHGKSLEWIGLIIPSNGGTNYNQKFKDKASLTVDKSSSTAYMELLSLTSEDSAVYYCARPSYYGSRNYYAMDYWGQGTSVTVSS

SEQ ID NO: 53 - protein, 245 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENGGGGSGGGGSDIVLTQSPASLAVSLGQRATISCRASESVDNSGFSFMNWFQQKPGQPPKLLIYAASNQGSGVPARFSGSGSETDFSLNIHPMEEDDTAVYFCQQSKEVPWTFGGGTKLEIK

SEQ ID NO: 54 - protein, 99 aa, Homo sapiens
SASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 55 - protein, 107 aa, Homo sapiens [sequence omitted]

SEQ ID NO: 56 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 117 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVS

SEQ ID NO: 58 - DNA, 1515 bp, Homo sapiens
atggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtga

SEQ ID NO: 59 - DNA, 972 bp, Homo sapiens
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataa

SEQ ID NO: 60 - DNA, 369 bp, Homo sapiens
cagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaa

SEQ ID NO: 61 - DNA, 372 bp, Homo sapiens
cagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaat

SEQ ID NO: 62 - DNA, 372 bp, Homo sapiens
cagctactatttaataaaacaaaatctgtagaattcacgtttggtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaat

SEQ ID NO: 63 - DNA, 699 bp, Homo sapiens
ctcgagccgaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 64 - DNA, 1128 bp, Homo sapiens
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatctcgagccgaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 65 - DNA, 15 bp, Homo sapiens
ggcggcggcggatcc

SEQ ID NO: 66 - DNA, 30 bp, Homo sapiens
ggaggtggtggatctggaggtggaggtagc

SEQ ID NO: 67 - DNA, 297 bp, Homo sapiens
tcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtg

SEQ ID NO: 68 - DNA, 696 bp, Homo sapiens
gagcccaagagctgcgacaagacccacacctgccccccctgcccagccccagaggcagcgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 69 - DNA, 993 bp, Homo sapiens
tcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagaggcagcgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 70 - DNA, 321 bp, Homo sapiens
cgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 71 - DNA, 1452 bp, Homo sapiens
atgtggcccctggtagcggcgctgttgctgggctcggcgtgctgcggatcagctcagctactatttaataaaacaaaatctgtagaattcacgtttggtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatggaggtggtggatctggaggtggaggtagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagaggcagcgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaagtga

SEQ ID NO: 72 - DNA, 780 bp, Homo sapiens
atgtggcccctggtagcggcgctgttgctgggctcggcgtgctgcggatcagctcagctactatttaataaaacaaaatctgtagaattcacgtttggtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatggaggtggtggatctggaggtggaggtagccgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgctga

SEQ ID NO: 73 - DNA, 1452 bp, Homo sapiens
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatggaggtggtggatctggaggtggaggtagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagaggcagcgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaagtga

SEQ ID NO: 74 - DNA, 780 bp, Homo sapiens
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatggaggtggtggatctggaggtggaggtagccgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgctga

SEQ ID NO: 75 - DNA, 1758 bp, Homo sapiens
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcaggcggcggcggatccagcgctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcggcggcggcggctccggcggcggcggatccagcgctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagaggcagcgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaagtga

SEQ ID NO: 76 - DNA, 1095 bp, Homo sapiens
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcaggcggcggcggatcccgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgcggcggcggcggctccggcggcggcggatcccgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgctga

SEQ ID NO: 77 - DNA, 1782 bp, Homo sapiens
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatgaggtgcaattggtggaaagcggcggaggactggtgcagcccggcagaagcctgagactgagctgcgccgccagcggcttcaccttcgacgactacgccatgcactgggtccgccaggcccctggcaagggactggaatgggtgtccgccatcacctggaacagcggccacatcgactacgccgacagcgtggaaggccggttcaccatcagccgggacaacgccaagaacagcctgtacctgcagatgaacagcctgcgggccgaggacaccgccgtgtactactgcgccaaggtgtcctacctgagcaccgccagcagcctggactactggggccagggcacactggtcacagtcagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaagtga

SEQ ID NO: 78 - DNA, 1071 bp, Homo sapiens
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatgatatccagatgacccagagccccagcagcctgagcgccagcgtgggcgacagagtgaccatcacctgtcgggccagccagggcatccggaactacctggcctggtatcagcagaagcccggcaaggcccccaagctgctgatctacgccgccagcaccctgcagagcggcgtgccaagcagattcagcggcagcggctccggcaccgacttcaccctgaccatcagcagcctgcagcccgaggacgtggccacctactactgccagcggtacaacagagccccctacaccttcggccagggcaccaaggtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgctga

SEQ ID NO: 79 - DNA, 1812 bp, Homo sapiens
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatggaggtggtggatctggaggtggaggatccgaggtccaattggtggaaagcggcggaggactggtgcagcccggcagaagcctgagactgagctgcgccgccagcggcttcaccttcgacgactacgccatgcactgggtccgccaggcccctggcaagggactggaatgggtgtccgccatcacctggaacagcggccacatcgactacgccgacagcgtggaaggccggttcaccatcagccgggacaacgccaagaacagcctgtacctgcagatgaacagcctgcgggccgaggacaccgccgtgtactactgcgccaaggtgtcctacctgagcaccgccagcagcctggactactggggccagggcacactggtcacagtcagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaagtga

SEQ ID NO: 80 - DNA, 1101 bp, Homo sapiens
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatggaggtggtggatctggaggtggaggatccgatatccagatgacccagagccccagcagcctgagcgccagcgtgggcgacagagtgaccatcacctgtcgggccagccagggcatccggaactacctggcctggtatcagcagaagcccggcaaggcccccaagctgctgatctacgccgccagcaccctgcagagcggcgtgccaagcagattcagcggcagcggctccggcaccgacttcaccctgaccatcagcagcctgcagcccgaggacgtggccacctactactgccagcggtacaacagagccccctacaccttcggccagggcaccaaggtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgctga

SEQ ID NO: 81 - DNA, 1353 bp, Homo sapiens
gaggtccaattggtggaaagcggcggaggactggtgcagcccggcagaagcctgagactgagctgcgccgccagcggcttcaccttcgacgactacgccatgcactgggtccgccaggcccctggcaagggactggaatgggtgtccgccatcacctggaacagcggccacatcgactacgccgacagcgtggaaggccggttcaccatcagccgggacaacgccaagaacagcctgtacctgcagatgaacagcctgcgggccgaggacaccgccgtgtactactgcgccaaggtgtcctacctgagcaccgccagcagcctggactactggggccagggcacactggtcacagtcagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 82 - DNA, 642 bp, Homo sapiens
gatatccagatgacccagagccccagcagcctgagcgccagcgtgggcgacagagtgaccatcacctgtcgggccagccagggcatccggaactacctggcctggtatcagcagaagcccggcaaggcccccaagctgctgatctacgccgccagcaccctgcagagcggcgtgccaagcagattcagcggcagcggctccggcaccgacttcaccctgaccatcagcagcctgcagcccgaggacgtggccacctactactgccagcggtacaacagagccccctacaccttcggccagggcaccaaggtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 83 - DNA, 360 bp, Homo sapiens
gaggtccaattggtggaaagcggcggaggactggtgcagcccggcagaagcctgagactgagctgcgccgccagcggcttcaccttcgacgactacgccatgcactgggtccgccaggcccctggcaagggactggaatgggtgtccgccatcacctggaacagcggccacatcgactacgccgacagcgtggaaggccggttcaccatcagccgggacaacgccaagaacagcctgtacctgcagatgaacagcctgcgggccgaggacaccgccgtgtactactgcgccaaggtgtcctacctgagcaccgccagcagcctggactactggggccagggcacactggtcacagtcagc

SEQ ID NO: 84 - DNA, 15 bp, Homo sapiens
gactacgccatgcac

SEQ ID NO: 85 - DNA, 51 bp, Homo sapiens
gccatcacctggaacagcggccacatcgactacgccgacagcgtggaaggc

SEQ ID NO: 86 - DNA, 36 bp, Homo sapiens
gtgtcctacctgagcaccgccagcagcctggactac

SEQ ID NO: 87 - DNA, 321 bp, Homo sapiens
gatatccagatgacccagagccccagcagcctgagcgccagcgtgggcgacagagtgaccatcacctgtcgggccagccagggcatccggaactacctggcctggtatcagcagaagcccggcaaggcccccaagctgctgatctacgccgccagcaccctgcagagcggcgtgccaagcagattcagcggcagcggctccggcaccgacttcaccctgaccatcagcagcctgcagcccgaggacgtggccacctactactgccagcggtacaacagagccccctacaccttcggccagggcaccaaggtggaaatcaag

SEQ ID NO: 88 - DNA, 33 bp, Homo sapiens
cgggccagccagggcatccggaactacctggcc

SEQ ID NO: 89 - DNA, 21 bp, Homo sapiens
gccgccagcaccctgcagagc

SEQ ID NO: 90 - DNA, 27 bp, Homo sapiens
cagcggtacaacagagccccctacacc

SEQ ID NO: 91 - DNA, 735 bp, Homo sapiens
cagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatgaggtgcaattggtggaaagcggcggaggactggtgcagcccggcagaagcctgagactgagctgcgccgccagcggcttcaccttcgacgactacgccatgcactgggtccgccaggcccctggcaagggactggaatgggtgtccgccatcacctggaacagcggccacatcgactacgccgacagcgtggaaggccggttcaccatcagccgggacaacgccaagaacagcctgtacctgcagatgaacagcctgcgggccgaggacaccgccgtgtactactgcgccaaggtgtcctacctgagcaccgccagcagcctggactactggggccagggcacactggtcacagtcagctca

SEQ ID NO: 92 - DNA, 693 bp, Homo sapiens
cagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatgatatccagatgacccagagccccagcagcctgagcgccagcgtgggcgacagagtgaccatcacctgtcgggccagccagggcatccggaactacctggcctggtatcagcagaagcccggcaaggcccccaagctgctgatctacgccgccagcaccctgcagagcggcgtgccaagcagattcagcggcagcggctccggcaccgacttcaccctgaccatcagcagcctgcagcccgaggacgtggccacctactactgccagcggtacaacagagccccctacaccttcggccagggcaccaaggtggaaatcaag

SEQ ID NO: 93 - DNA, 765 bp, Homo sapiens
cagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatggaggtggtggatctggaggtggaggatccgaggtccaattggtggaaagcggcggaggactggtgcagcccggcagaagcctgagactgagctgcgccgccagcggcttcaccttcgacgactacgccatgcactgggtccgccaggcccctggcaagggactggaatgggtgtccgccatcacctggaacagcggccacatcgactacgccgacagcgtggaaggccggttcaccatcagccgggacaacgccaagaacagcctgtacctgcagatgaacagcctgcgggccgaggacaccgccgtgtactactgcgccaaggtgtcctacctgagcaccgccagcagcctggactactggggccagggcacactggtcacagtcagctca

SEQ ID NO: 94 - DNA, 723 bp, Homo sapiens
cagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatggaggtggtggatctggaggtggaggatccgatatccagatgacccagagccccagcagcctgagcgccagcgtgggcgacagagtgaccatcacctgtcgggccagccagggcatccggaactacctggcctggtatcagcagaagcccggcaaggcccccaagctgctgatctacgccgccagcaccctgcagagcggcgtgccaagcagattcagcggcagcggctccggcaccgacttcaccctgaccatcagcagcctgcagcccgaggacgtggccacctactactgccagcggtacaacagagccccctacaccttcggccagggcaccaaggtggaaatcaag

SEQ ID NO: 95 - DNA, 1356 bp, Homo sapiens
gaggtccaattggtggaaagcggcggaggactggtgcagcccggcagaagcctgagactgagctgcgccgccagcggcttcaccttcgacgactacgccatgcactgggtccgccaggcccctggcaagggactggaatgggtgtccgccatcacctggaacagcggccacatcgactacgccgacagcgtggaaggccggttcaccatcagccgggacaacgccaagaacagcctgtacctgcagatgaacagcctgcgggccgaggacaccgccgtgtactactgcgccaaggtgtcctacctgagcaccgccagcagcctggactactggggccagggcacactggtcacagtcagctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 96 - DNA, 642 bp, Homo sapiens; identical to SEQ ID NO: 82

SEQ ID NO: 97 - DNA, 1818 bp, Homo sapiens
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatggaggtggtggatctggaggtggaggatccgaggtgcaattggagcagagcggccctgtgctggtgaagcccggcaccagcatgaagatcagctgcaagaccagcggctacagcttcaccggctacaccatgtcctgggtgcgccagagccacggcaagagcctggaatggatcggcctgatcatccccagcaacggcggcaccaactacaaccagaagttcaaggacaaggccagcctgaccgtggacaagagcagcagcaccgcctacatggaactgctgtccctgaccagcgaggacagcgccgtgtactactgcgccagacccagctactacggcagccggaactactacgccatggactactggggccagggcaccagcgtgaccgtcagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagaggcagcgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaagtga

SEQ ID NO: 98 - DNA, 1113 bp, Homo sapiens
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatggaggtggtggatctggaggtggaggatccgatatcgtgctgacccaatctccagcttctttggctgtgtctctagggcagagggccaccatctcctgcagggccagcgaaagtgttgataattctggctttagttttatgaactggttccaacagaaaccaggacagccacccaaactcctcatctatgctgcatccaaccaaggatccggggtccctgccaggtttagtggcagtgggtctgagacagacttcagcctcaacatccatcctatggaggaggatgatactgcagtgtatttctgtcagcaaagtaaggaggttccttggacgttcggtggaggcaccaagctggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgctga

SEQ ID NO: 99 - DNA, 1359 bp, Homo sapiens
gaggtgcaattggagcagagcggccctgtgctggtgaagcccggcaccagcatgaagatcagctgcaagaccagcggctacagcttcaccggctacaccatgtcctgggtgcgccagagccacggcaagagcctggaatggatcggcctgatcatccccagcaacggcggcaccaactacaaccagaagttcaaggacaaggccagcctgaccgtggacaagagcagcagcaccgcctacatggaactgctgtccctgaccagcgaggacagcgccgtgtactactgcgccagacccagctactacggcagccggaactactacgccatggactactggggccagggcaccagcgtgaccgtcagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagaggcagcgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 100 - DNA, 654 bp, Homo sapiens
gatatcgtgctgacccaatctccagcttctttggctgtgtctctagggcagagggccaccatctcctgcagggccagcgaaagtgttgataattctggctttagttttatgaactggttccaacagaaaccaggacagccacccaaactcctcatctatgctgcatccaaccaaggatccggggtccctgccaggtttagtggcagtgggtctgagacagacttcagcctcaacatccatcctatggaggaggatgatactgcagtgtatttctgtcagcaaagtaaggaggttccttggacgttcggtggaggcaccaagctggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 101 - DNA, 366 bp, Homo sapiens
gaggtgcaattggagcagagcggccctgtgctggtgaagcccggcaccagcatgaagatcagctgcaagaccagcggctacagcttcaccggctacaccatgtcctgggtgcgccagagccacggcaagagcctggaatggatcggcctgatcatccccagcaacggcggcaccaactacaaccagaagttcaaggacaaggccagcctgaccgtggacaagagcagcagcaccgcctacatggaactgctgtccctgaccagcgaggacagcgccgtgtactactgcgccagacccagctactacggcagccggaactactacgccatggactactggggccagggcaccagcgtgaccgtcagc

SEQ ID NO: 102 - DNA, 15 bp, Homo sapiens
ggctacaccatgtcc

SEQ ID NO: 103 - DNA, 51 bp, Homo sapiens
ctgatcatccccagcaacggcggcaccaactacaaccagaagttcaaggac

SEQ ID NO: 104 - DNA, 42 bp, Homo sapiens
cccagctactacggcagccggaactactacgccatggactac

SEQ ID NO: 105 - DNA, 333 bp, Homo sapiens
gatatcgtgctgacccaatctccagcttctttggctgtgtctctagggcagagggccaccatctcctgcagggccagcgaaagtgttgataattctggctttagttttatgaactggttccaacagaaaccaggacagccacccaaactcctcatctatgctgcatccaaccaaggatccggggtccctgccaggtttagtggcagtgggtctgagacagacttcagcctcaacatccatcctatggaggaggatgatactgcagtgtatttctgtcagcaaagtaaggaggttccttggacgttcggtggaggcaccaagctggaaatcaag

SEQ ID NO: 106 - DNA, 45 bp, Homo sapiens
agggccagcgaaagtgttgataattctggctttagttttatgaac

SEQ ID NO: 107 - DNA, 21 bp, Homo sapiens
gctgcatccaaccaaggatcc

SEQ ID NO: 108 - DNA, 27 bp, Homo sapiens
cagcaaagtaaggaggttccttggacg

SEQ ID NO: 109 - DNA, 771 bp, Homo sapiens
cagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatggaggtggtggatctggaggtggaggatccgaggtgcaattggagcagagcggccctgtgctggtgaagcccggcaccagcatgaagatcagctgcaagaccagcggctacagcttcaccggctacaccatgtcctgggtgcgccagagccacggcaagagcctggaatggatcggcctgatcatccccagcaacggcggcaccaactacaaccagaagttcaaggacaaggccagcctgaccgtggacaagagcagcagcaccgcctacatggaactgctgtccctgaccagcgaggacagcgccgtgtactactgcgccagacccagctactacggcagccggaactactacgccatggactactggggccagggcaccagcgtgaccgtcagctca

SEQ ID NO: 110 - DNA, 735 bp, Homo sapiens
cagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatggaggtggtggatctggaggtggaggatccgatatcgtgctgacccaatctccagcttctttggctgtgtctctagggcagagggccaccatctcctgcagggccagcgaaagtgttgataattctggctttagttttatgaactggttccaacagaaaccaggacagccacccaaactcctcatctatgctgcatccaaccaaggatccggggtccctgccaggtttagtggcagtgggtctgagacagacttcagcctcaacatccatcctatggaggaggatgatactgcagtgtatttctgtcagcaaagtaaggaggttccttggacgttcggtggaggcaccaagctggaaatcaag

SEQ ID NO: 111 - DNA, 297 bp, Homo sapiens
tcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtt

SEQ ID NO: 112 - DNA, 321 bp, Homo sapiens; identical to SEQ ID NO: 70

SEQ ID NO: 113 - DNA, 696 bp, Homo sapiens
gagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 114 - DNA, 351 bp, Homo sapiens
cagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttca

SEQ ID NO: 115 - protein, 387 aa, Homo sapiens
MPVPASWPHPPGPFLLLTLLLGLTEVAGEEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPSAPVVLGPAARTTPEHTVSFTCESHGFSPRDITLKWFKNGNELSDFQTNVDPTGQSVAYSIRSTARVVLDPWDVRSQVICEVAHVTLQGDPLRGTANLSEAIRVPPTLEVTQQPMRVGNQVNVTCQVRKFYPQSLQLTWSENGNVCQRETASTLTENKDGTYNWTSWFLVNISDQRDDVVLTCQVKHDGQLAVSKRLALEVTVHQKDQSSDATPGPASSLTALLLIAVLLGPIYVPWKQKT

SEQ ID NO: 116 - protein, 233 aa, Homo sapiens
LEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 357 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENLEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - DNA, 699 bp, Homo sapiens
ctcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 119 - DNA, 1128 bp, Homo sapiens
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatctcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 120 - protein, 15 aa, Artificial Sequence
  Linker sequence
EPKSCGGGGSGGGGS

SEQ ID NO: 121 - DNA, 45 bp, Artificial Sequence
  Linker sequence
gagcccaagagctgcggcggcggcggctccggcggcggcggatcc

SEQ ID NO: 122 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - DNA, 696 bp, Homo sapiens
gagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 124 - DNA, 297 bp, Homo sapiens
agcgctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtg

SEQ ID NO: 125 - DNA, 30 bp, Artificial Sequence
  Linker sequence
ggcggcggcggctccggcggcggcggatcc

SEQ ID NO: 126 - DNA, 30 bp, Artificial Sequence
  Linker Sequence
ggaggtggtggatctggaggtggaggatcc

SEQ ID NO: 127 - DNA, 360 bp, Homo sapiens
gaggtgcaattggtggaaagcggcggaggactggtgcagcccggcagaagcctgagactgagctgcgccgccagcggcttcaccttcgacgactacgccatgcactgggtccgccaggcccctggcaagggactggaatgggtgtccgccatcacctggaacagcggccacatcgactacgccgacagcgtggaaggccggttcaccatcagccgggacaacgccaagaacagcctgtacctgcagatgaacagcctgcgggccgaggacaccgccgtgtactactgcgccaaggtgtcctacctgagcaccgccagcagcctggactactggggccagggcacactggtcacagtcagc